(19) United States
(12) Patent Application Publication
(10) Pub. No.: US 2011/0189174 A1
(43) Pub. Date: Aug. 4, 2011

(54) COMPOSITIONS AND METHODS FOR TREATING, REDUCING, AMELIORATING, ALLEVIATING, OR INHIBITING PROGRESSION OF, PATHOGENIC OCULAR NEOVASCULARIZATION

(76) Inventors: Afshin Shafiee, Rochester, NY (US); Keith W. Ward, Ontario, NY (US)

(21) Appl. No.: 13/009,894

(22) Filed: Jan. 20, 2011

Related U.S. Application Data
(60) Provisional application No. 61/300,138, filed on Feb. 1, 2010.

(51) Int. Cl. A61K 31/506; A61K 39/395; C07D 471/04; A61K 31/713; A61P 27/02; A61P 29/00

(52) U.S. Cl. 424/133.1; 424/158.1; 514/256; 514/274; 544/322; 514/44 A

(57) ABSTRACT

A composition for treating, reducing, ameliorating, alleviating, or inhibiting the progression of, pathological ocular neovascularization comprises an integrin or vitronectin receptor antagonist having any one of Formulae I-XI, as defined herein. The composition can further comprise a VEGF inhibitor. Such composition is administered to an ocular environment by a method such as topical application, periocular injection, intravitreal injection, or intravitreal implantation. The composition can be administered alone or in combination with another procedure chosen to enhance the outcome of the treatment.

### CROSS REFERENCE

This application claims the benefit of Provisional Patent Application No. 61/300,138 filed Feb. 1, 2010 which is incorporated by reference herein.

### BACKGROUND OF THE INVENTION

The present invention relates to compositions and methods for treating, reducing, ameliorating, alleviating, or inhibiting the progression of, pathogenic ocular neovascularization. In particular, the present invention relates to compositions that comprise an integrin receptor antagonist, or a vitronectin receptor antagonist, and to methods for the treatment, reduction, amelioration, alleviation, or inhibition of the progression, of pathogenic ocular neovascularization using such compositions. In addition, the present invention relates to compositions and methods using such integrin or vitronectin receptor antagonist for treating, reducing, ameliorating, alleviating, or inhibiting the progression of, pathogenic ocular neovascularization that has etiology in inflammation.

Ophthalmic conditions may be classified as front-of-the-eye diseases, such as corneal edema, anterior uveitis, pterygium, corneal diseases, opacification with an exudative or inflammatory component, conjunctivitis, and allergy- and laser-induced exudation, or back-of-the-eye diseases such as exudative macular degeneration, macular edema, diabetic retinopathy, age-related macular degeneration, and retinopathy of prematurity. Back-of-the-eye diseases comprise the largest number of causes for vision loss.

Many front-of-the-eye or back-of-the-eye diseases manifest pathological growth of new blood vessels (pathogenic neovascularization or angiogenesis) in various tissues of the eye, including the cornea, iris, retina, and choroid. The consequences of neovascularization within these delicate ocular tissues are fibrosis, exudation, and/or hemorrhage that are responsible for catastrophic vision loss in many common eye diseases.

Corneal neovascularization is characterized by invasion of vascular capillaries from the limbal vascular plexus into normally avascular cornea. In some cases, corneal neovascularization is associated with a decrease in visual acuity and often is a consequence of mechanical or chemical injury, or secondary to infection.

Iris neovascularization is characterized by the formation of leaky new blood vessels on the anterior surface of the iris and in the chamber angle recess. In the late stage of the disease, the vessels are enlarged and are accompanied by fibrous tissue, hence occluding the angle and causing the secondary neovascular glaucoma, a condition characterized by high intraocular pressure, neovascularization of the iris and trabecular meshwork. Iris neovascularization and consequent neovascular glaucoma respond poorly to therapies and are frequent causes of blindness and enucleation. Iris neovascularization is associated with a variety of systemic and ocular diseases and secondary to trauma or therapies including surgery and radiation. Central retinal vein occlusion and diabetes mellitus are considered as leading causes of iris neovascularization.

Neovascularization of the retina involves the growth of new capillaries from the vessels that arise from the optic disk or inner retina. In the later stage, vision loss may occur due the development of various complications including scarring, tractional detachment of the retina, and hemorrhage.

Retinal neovascularization is associated with a variety of ocular and systemic diseases. Among those, diabetes mellitus, retinopathy of prematurity, central retinal vein occlusion, branch retinal vein occlusion, and sickle cell disease are most frequently associated with retinal neovascularization. Diabetic retinopathy is the leading cause of blindness in adults between the ages of 18 to 72 who suffer from diabetes mellitus. In the early stage, the vasculature of the retina is increasingly obstructed by the adhesion of cells involved in immunological response, such as leucocytes, on molecules, such as intercellular adhesion molecule-1 (“ICAM-1”) or vascular cell adhesion molecule-1 (“VCAM-1”), which are overexpressed on the endothelial layer of inflammed vasculature. The vasculature obstruction results in ischemia and leads to hypoxia condition in the surrounding tissues, especially the retina. In response to such a condition, new blood vessels begin to proliferate uncontrollably. These new blood vessels are typically leaky, resulting in fluid accumulation under the retina and eventually the vision-threatening condition known as macular edema. (See; e.g., A. P. Adamis, British J. Ophthalmol., Vol. 86, 363 (2002); S. Ishida et al., Invest. Ophthalmol. & Visual Sci., Vol. 44, No. 5, 2155 (2003).) Vascular endothelial growth factor (“VEGF”), a hypoxia-induced proinflammatory angiogenic factor, has been found at elevated levels in the diabetic retina during both the nonproliferative and proliferative stage. VEGF also induces the expression of ICAM-1 and VCAM-1 on endothelial cells. (I. Kim et al., Biol. Chem., Vol. 276, No. 10, 7614 (2001).) In addition, experimental investigations in animals have shown that mRNA expression for the proinflammatory cytokines IL-1 (interleukin-1) and TNF-α (tumor necrosis factor-α) is increased in the retina early in the course of diabetes, and moreover, inhibition of TNF-α has demonstrated beneficial effects in the prevention of diabetic retinopathy. (J. F. Navarro and C. Mora, Nephrol. Dial. Transplant, Vol. 20, 2601 (2005).)

Choroidal neovascularization (“CNV”) is characterized by an invasion of new blood vessels through Bruch's membrane. The consequence of CNV is severe and irreversible vision loss. CNV is associated with a variety of ocular diseases including degenerative conditions, inflammatory or infectious diseases and trauma. Age-related macular degeneration (“AMD”), angioid streaks, pathological myopia, ocular histoplasmosis syndrome, sarcoidosis and chronic uveitis are just a few examples of ocular conditions with choroidal neovascularization as a significant underlining pathological change.

Neovascularization or angiogenesis is mediated by, among other things, the infiltration and adhesion of endothelial cells and smooth muscle cells. The process is believed to proceed in any one of three ways: The vessels can sprout from pre-existing vessels, de-novo development of vessels can arise from precursor cells (vasculogenesis), or existing small vessels can enlarge in diameter. Blood et al., Bioch. Biophys. Acta, Vol. 1032, 89-118 (1990). Cell adhesion is facilitated by cell adhesion receptors expressed on their surface, which can be grouped into four superfamilies: integrins, immunoglobulin supergene, selectin, and cadherin families.

The integrins comprise a family of transmembrane heterodimeric glycoproteins that mediate cell-cell adhesion and cell-extracellular matrix adhesion. Each integrin receptor molecule comprises an α subunit and a β subunit noncovalently bound together. To date, eighteen α and eight β subunits have been identified. These α and β subunits associate with each other in various combinations to produce 22 integrins identified thus far.

The vitronectin surface receptors are a subset of the integrin receptor family and comprise αᵥβ₁, αᵥβ₃ and αᵥβ₅. Horton, Int. J. Exp. Pathol., Vol. 71, 741-759 (1990). αᵥβ₁ binds fibronectin and vitronectin. αᵥβ₃ binds a large variety of ligands, including fibrin, fibrinogen, laminin, thrombospondin, vitronectin, von Willebrand's factor, osteospontin and bone sialoprotein I. αᵥβ₅ binds vitronectin. Inhibition of cell adhesion in vitro using monoclonal antibodies immunospecific for various integrin a or subunits have implicated αᵥβ₃ in cell adhesion of a variety of cell types including microvascular endothelial cells. C. M. Davis et al., J. Cell. Biol., Vol. 51, 206-218 (1993).

The current treatment for many forms of ocular neovascularization involves photocoagulation or cryotherapy. Pan-retinal or focal photocoagulation is current standard therapy for diabetic retinopathy. It is partially effective in reducing the rate of vision loss in patients with diabetic retinopathy. Photocoagulation is also a destructive treatment with unwanted side effects, such as CNV, subretinal fibrosis, photocoagulation scar expansion, and inadvertent foveolar burns, which can cause loss of central visual acuity and scotoma formation. Patients with good visual acuity are less likely to recognize the benefits from this aggressive treatment and more likely to notice its side effects, which can include some loss of central and peripheral vision, and a reduction in color and night vision.

In 2006, the US Food and Drug Administration (“FDA”) approved the intravitreal injection of ranibizumab (or Lucentis®, developed by Genentech) for the treatment of wet AMD. Ranibizumab is a monoclonal antibody fragment against VEGF-A and binds to all VEGF-A isoforms. In large phase III clinical trials, 95% of the patients treated with ranibizumab maintained visual acuity at 1 year, and 40% of the patients demonstrated improvement in vision of at least 3 lines. According to the package insert, side effects of intravitreal injection of ranibizumab include conjunctival hemorrhage, eye pain, vitreous floaters, increased intraocular pressure, and intraocular inflammation. Intravitreal injection of bevacizumab (or Avastin®, developed by Genentech) has been tested in studies for the treatment of choroidal neovascularization and macular edema. See; e.g., R. F. Spaide et al., Retina, Vol. 26, 383 (2006); and D. Iturralde et al., Retina, Vol. 26, 279 (2006). The treated eyes had significant decrease in macular thickness, macular edema and significant improvement in visual acuity. Bevacizumab is the full length antibody against VEGF-A, the parent antibody of ranibizumab, and was approved for cancers that are metastatic. The medium to long term safety of intravitreal injection of bevacizumab for the treatment of macular degeneration is unknown at this time.

Glucocorticoids (also referred to herein as “corticosteroids”) have been under investigation for use as a local therapeutic treatment for diabetic retinopathy. (See; e.g., M. A. Speicher et al., Expert Opinion on Emerging Drugs, Vol. 8, No. 1, 239 (2003); E. A. Felinski and D. A. Antonetti, Curr. Eye Research, Vol. 30, No. 11, (49 (2005); and G. M. Corner and T. A. Ciulla, Expert Opinion on Emerging Drugs, Vol. 10, No. 2, 441 (2005).) Intravitreal injection of corticosteroids (especially triamcinolone acetonide) has been investigated as a treatment for diabetic macular edema (see; e.g., V. Vasumathy et al., Ophthalmic Practice, Vol. 54, No. 2, 133 (2006); P. Massin et al., Ophthalmology, Vol. 111, No. 2, 218 (2004)). A Phase-III clinical trial of periocular injection of triamcinolone acetonide as adjunct therapy to photodynamic therapy (“PDT”) for neovascular AMD was completed by Johns Hopkins University School of Medicine and Oregon Health Science University in 2006 (see, www.clinicaltrials.gov). However, steroidal drugs can have side effects that threaten the overall health of the patient. For example, it is known that chronic use of certain glucocorticoids has a great potential for elevating intraocular pressure (“IOP”). In addition, use of corticosteroids is also known to increase the risk of cataract formation in a dose- and duration-dependent manner. Once cataracts develop, they may progress despite discontinuation of corticosteroid therapy.

Chronic administration of glucocorticoids also can lead to drug-induced osteoporosis by suppressing intestinal calcium absorption and inhibiting bone formation. Other adverse side effects of chronic administration of glucocorticoids include hypertension, hyperglycemia, hyperlipidemia (increased levels of triglycerides) and hypercholesterolemia (increased levels of cholesterol) because of the effects of these drugs on the body metabolic processes.

Therefore, there is a continued need to provide pharmaceutical compounds and compositions to treat, reduce, ameliorate, or inhibit the progression of pathogenic ocular neovascularization. It is also desirable to provide such compounds or compositions that cause a lower level of at least an adverse side effect than at least a prior-art method used to treat, reduce, ameliorate, or inhibit the progression of, the same condition.

### SUMMARY OF THE INVENTION

In general, the present invention provides pharmaceutical compounds and compositions for treating, reducing, ameliorating, or inhibiting the progression of, pathological ocular neovascularization in a subject.

In one aspect, the present invention also provides a method for treating, reducing, ameliorating, or inhibiting the progression of, pathological ocular neovascularization in a subject by using such compounds or compositions.

In another aspect, the present invention provides pharmaceutical compounds and compositions for treating, reducing, ameliorating, or inhibiting the progression of, a pathological ocular condition or disorder resulting from, or having an etiology in, ocular neovascularization in a subject.

In still another aspect, a compound or composition of the present invention causes a lower level of at least an adverse side effect than at least a prior-art glucocorticoid used to treat, reduce, or ameliorate the same condition or disorder.

In yet another aspect, such a condition or disorder is selected from the group consisting of diabetic retinopathy (“DR”), age-related macular degeneration (“AMD,” including dry and wet AMD), diabetic macular edema (“DME”), retinal detachment, posterior uveitis, corneal neovascularization, iris neovascularization, and combinations thereof.

In a further aspect, a pharmaceutical compound or a composition of the present invention comprises an integrin receptor antagonist. In one embodiment, a pharmaceutical compound or a composition of the present invention comprises a vitronectin receptor antagonist.

In still another aspect, a pharmaceutical composition of the present invention comprises an integrin receptor antagonist and a VEGF antagonist. In one embodiment, a pharmaceutical composition of the present invention comprises a vitronectin receptor antagonist and a VEGF antagonist. In another embodiment, such a VEGF antagonist comprises antagonist to a VEGF-A isoform.

In yet another aspect, such a VEGF antagonist comprises an antibody to VEGF-A.

In yet another aspect, a pharmaceutical composition of the present invention comprises an ophthalmic topical formulation; injectable formulation; or implantable formulation, system, or device.

In a further aspect, said at least an adverse side effect is demonstrated in vitro or in vivo.

In another aspect, the present invention provides a method for treating, reducing, ameliorating, alleviating, or inhibiting the progression of, pathological ocular neovascularization. The method comprises administering a composition comprising an antagonist to an integrin receptor or vitronectin receptor, a prodrug, a pharmaceutically acceptable salt, ester, hydrate, solvate, or clathrate thereof into a subject in need of such treatment, reduction, amelioration, alleviation, or inhibition.

In still another aspect, the method further comprises performing an additional procedure in the subject to enhance the treatment, reduction, amelioration, alleviation, or inhibition of the progression of the condition or disorder.

Other features and advantages of the present invention will become apparent from the following detailed description and claims.

### BRIEF DESCRIPTION OF THE DRAWINGS

FIG. 1 shows the effects of integrin antagonists and echistatin on PMA-induced HREC network formation in fibrin gel. Each data point represents the mean for a treatment group (n=3; 6 for echistatin and controls) and bars represent standard error of the means. Data were analyzed using a One-way ANOVA-Dunnett's test on raw data. “*” denotes p<0.05, compared to the PMA control.

FIG. 2 shows representative images of network formation of HREC in a fibrin gel. The following conditions are shown: vehicle control (0.1% DMSO), PMA 25 ng/ml, PMA+Echistatin, PMA+BOL-303049-X-1 and 10 nM, PMA+BOL-303051-X 1 and 10 nM, PMA+BOL-303054-X 1 and 10 nM.

FIG. 3 shows incidences of grade IV lesions (%) in eyes treated with vehicle, BOL-303050-X, BOL-303054-X and BOL-303055-X. Test agents were injected intravitreally into both eyes on days 1 and 15 and lesions were scored on days 22, 29 and 37 post-injection. The number of grade IV lesions out of the total number of lesions is given between parentheses. “*” denotes P<0.05 versus vehicle control group using the Fisher's exact probability test (2-tail).

FIG. 4 shows change in IOP from baseline in eyes treated with Lucentis, vehicle, and BOL-303055-X (top panel) or BOL-303050-X (bottom panel). Test agents were injected (Inj) intravitreally into both eyes on days 1 and 15 and IOP was measured at baseline (B) as well as on days 1, 3, 7, 15, 31 and 38. Analysis was performed using a two-way ANOVA followed by the contrast procedure on data transformed by the power of 0.4 (right panel and raw data (left panel). On the right panel: Factor 1 (treatment) F=0.979, NS; Factor 2 (Time) F=11.67, p<0.0001; Factor 1*Factor 2, F=1.37, NS. On the left panel: Factor 1 (treatment) F=1.01, NS; Factor 2 (Time) F=8.60, p<0.0001; Factor 1*Factor 2, F=1.45, NS. Significance compared to the baseline of the same group is marked by *, significance compared to the BOL-3030YY-X vehicle control is marked by †.

FIG. 5 shows quantitative measurement of CNV area on the isolectin IB₄-labeled flat mounts. Data represent the means±SEM. One-way ANOVA was used to analyze the differences between groups followed by Tukey-Kramer test on Log-transformed data. Groups marked with different letters were statistically significantly different. The top panel uses the individual area of each lesion (n=24 for vehicle-treated group; n=18 for all other groups), whereas the bottom panel uses the total area summed from six lesions in each eye (n=4 for vehicle-treated group; n=3 for all other groups).

FIG. 6 shows Mean score of fluorescein leakage in different groups. Data represent the means±SEM. One-way ANOVA was used to analyze the differences between groups followed by Tukey-Kramer test on raw data. Groups marked with different letters were statistically significantly different. Lesion numbers are listed in Table 7. No sample was qualified for FA grading in BOL-303049-X-treated group.

FIG. 7 shows that the combination of BOL-303050-X and Avastin enhances activity of each compound alone on VEGF-induced HREC proliferation. A. Data are the means for each treatment group (n=4), and bars represent standard error of the means. Analysis was performed using a two-way ANOVA followed by the contrast procedure on raw data. The difference between compound dosages and Avastin dosages was significant (F=1088, P<0.001; F=158, P<0.001 respectively), as well as that between groups with or without VEGF (F=124, P<0.001). The interaction between Avastin and compound was statistically significant (F=5.2, p<0.001). There was no significant difference in the interaction between VEGF and the compound dose (F=2.24, p=0.13). Significance compared to the basal group is marked by * or ***; significance compared to the VEGF control is marked by **; significance compared to the control of the same group is marked by t; and significance compared to the same treatment group lacking BOL-303050-X is marked by . Data are the means of % inhibition for the three groups (n=4), and bars represent standard error of the means.

FIG. 8 shows that BOL-303050-X enhances the effect of Avastin on inhibiting VEGF-induced HREC proliferation. Data are the means for net inhibition OD values (subtracted from VEGF mean OD) in each group (n=4) multiplied by −1, and lines on top of bars represent standard error of the means. Analysis was performed using a general linear model custom test equivalent to the Scheffé's test to determine whether the net inhibition of the combination was significantly different from the sum of the net inhibitions obtained in the individual treatments. Significant differences above the sum of the individual value would indicate synergism; no significant differences would indicate additive effects, whereas significant differences below the sum of the individual values would indicate antagonistic effects. The cutoff (sum of effects for individual treatment) is represented in the figure by the doubly hatched bar and the interrupted line. A. Data obtained when BOL-303050-X was used at 1 nM. B. BOL-303050-X was tested at 10 nM.

FIG. 9 shows the individual effect of VEGF, echistatin and Avastin on HREC proliferation under basal conditions. Data are the means for each treatment group (n=4), and bars on top of bars and symbols represent standard error of the means. Analysis was performed using a one-way ANOVA comparing basal conditions to echistatin 2 nM, VEGF 10 ng/ml, and the dose range of Avastin®, which was followed by a Dunnett's test on raw data. The difference between basal and echistatin was significant (p<0.001), as well as that between basal and VEGF (p<0.001). Avastin® did not show a significant difference from the basal at any dose tested. Significance compared to the basal control is marked by an asterisk (*).

FIG. 10 shows the combination of Avastin and echistatin enhances activity of each compound alone on VEGF-induced HREC proliferation. Data are means for each treatment group (n=4), and lines on top of bars and symbols represent standard error of the means. Analysis was performed using a two-way ANOVA followed by the contrast procedure on raw data. The difference between groups with echistatin and those without was significant (F=58.5, p<0.0001). The Avastin® dose also showed a significant effect (F=6.1, p=0.0003). The interaction between echistatin and the Avastin dose was not statistically significant (F=0.37, NS). Significance compared to the control of the same group is marked by *, and significance compared to the same treatment group lacking echistatin is marked by †.

FIG. 11 shows that echistatin shows additive effects with Avastin at inhibiting VEGF-induced HREC proliferation. Data are the means for net inhibition (OD values subtracted from VEGF mean OD) in each group (n=4), and lines on top of bars represent standard error of the means. Analysis was performed using a general linear model custom test equivalent to the Scheffé's test to determine whether the net inhibition of the combination was significantly different from the sum of the net inhibitions obtained in the individual treatments. Significant differences above the sum of the individual values would indicate synergism; no significant differences would indicate additive effects, whereas significant differences below the sum of the individual values would indicate antagonistic effects. NS=not significantly different versus the sum of the individual treatments (represented in the figure by the doubly hatched bar and the interrupted line).

FIG. 12 shows that the combination of BOL-3030550X and Avastin enhances activity of each compound alone on VEGF-induced HREC proliferation. A. Data are the geometric means of OD for each treatment group (n=4), and lines on top of bars and symbols represent SE estimated by the Taylor series expansion. Analysis was performed using a three-way ANOVA followed by the contrast procedure on log-transformed data. The difference between groups with VEGF and those without was significant (F=1095, p<0.0001), as well as groups with BOL-303055-X and those without (F=1383, p<0.0001). Treatment with the dose range of Avastin® had a significant effect (F=58, p<0.0001). The interactions between VEGF and the Avastin® dose, BOL-303055-X and the Avastin® dose, and between VEGF and BOL-303055-X were all statistically significant (F=21, 9, and 75 respectively; p<0.0001 for each). The interaction of all three factors was also significant (F=6, p<0.0001). Significance compared to the control of the same group is marked by *, and significance compared to the same treatment group lacking BOL-303055-X is marked by †. B. Data are the means of % inhibition for the groups of VEGF+Avastin or VEGF+Avastin+BOL-303055-X.

FIG. 13 shows that BOL-303055-X shows synergistic effects with Avastin on inhibiting VEGF-induced HREC proliferation. Data are the means for net inhibition (OD values subtracted from VEGF mean OD) in each group (n=4), and lines on top of bars represent standard error of the means. Analysis was performed using a general linear model custom test equivalent to the Scheffé's test to determine whether the net inhibition of the combination was significantly different from the sum of the net inhibitions obtained in the individual treatments. Significant differences above the sum of the individual values would indicate synergism; no significant differences would indicate additive effects, whereas significant differences below the sum of the individual values would indicate antagonistic effects. NS=not significantly different versus the sum of the individual treatments (represented in the figure by the doubly colored bar and the interrupted line).

FIG. 14 shows that the combination of Lucentis and BOL-303055-X enhances activity of the compound alone on VEGF-induced HREC proliferation. Data are the means for each treatment group (n=4), and bars represent standard error of the means. Analysis was performed using a 3-way ANOVA followed by the contrast procedure on raw data. Factor 1 (VEGF) F=939.8, p<0.0001, factor 2 (Lucentis®) F=123.8, p<0.0001, factor 3 (dose of compound) F=254.5, p<0.0001. Factor 1*Factor 2, F=110.5, p<0.0001. Factor 1*Factor 3, F=3.3 p=0.009. Factor 1*Factor 3, F=3.3, p=0.009. Factor 2*Factor 3, F=2.1, p=0.08. Factor 1*Factor 2*Factor 3, F=1.7, p=0.14. Significance compared to the control of the same group is marked by *, and significance compared to the same treatment group lacking Lucentis® is marked by †.

FIG. 15 shows that the combination of Lucentis® and integrin antagonists enhanced activity of each of the five compounds alone on VEGF-induced HREC proliferation. Data are the means for each treatment group (n=4), and bars represent standard error of the means. Analysis was performed using a 3-way ANOVA followed by the contrast procedure on data elevated to the power 0.4. Factor 1 (VEGF), F=1263, p<0.0001. Factor 2 (Lucentis®), p<0.0001. Factor 3 (compounds), F=127.7, p<0.0001. Factor 1*Factor 2, F=42.1, p<0.0001. Factor 1*Factor 3, F=12, p<0.0001. Factor 2*Factor 3, F=2.48, p=0.04. Factor 1*Factor 2*Factor 3, F=1.35, p=0.25. Black bars represent the control of each group without the compound. Significance compared to the control of the same group is marked by *, and significance compared to the same treatment group lacking Lucentis® is marked by †.

FIG. 16 shows that BOL-303055-X at 0.1 nM exhibits a synergistic effect with Lucentis® to inhibit VEGF-induced HREC proliferation. Data are the means for net inhibition OD values (subtracted from VEGF mean OD) in each group (n=4), and lines on top of bars represent standard error of the means. Analysis was performed using a general linear model custom test equivalent to the Scheffé's test to determine whether the net inhibition of the combination was significantly different from the sum of the net inhibitions obtained in the individual treatments. Significant differences above the sum of the individual values would indicate synergism, no significant differences would indicate additive effects, whereas significant differences below the sum of the individual values would indicate antagonistic effects. NS=not significantly different versus the sum of the individual treatments (represented in the figure by the doubly hatched bar and the interrupted line.

FIG. 17 shows that at the concentration of 1 nM, BOL-303049-X shows synergistic effect with Lucentis® at inhibiting VEGF-induced HREC proliferation, while BOL-303050-X, BOL-303051-X, BOL-303054-X and BOL-303055-X show additive effects with Lucentis® at inhibiting VEGF-induced HREC proliferation. Data are the means for net inhibition OD values (subtracted from VEGF mean OD) in each group (n=4), and lines on top of bars represent standard error of the means. Analysis was performed using a general linear model custom test equivalent to the Scheffé's test to determine whether the net inhibition of the combination was significantly different from the sum of the net inhibitions obtained in the individual treatments. Significant differences above the sum of the individual values would indicate synergism, no significant differences would indicate additive effects, whereas significant differences below the sum of the individual values would indicate antagonistic effects. NS=not significantly different versus the sum of the individual treatments (represented in the figure by the doubly hatched bar and the interrupted line.

FIG. 18 shows a study design for Testing 3.

### DETAILED DESCRIPTION OF THE INVENTION

As used herein, the term “prodrug” means a pharmacologically inactive derivative of a drug molecule that requires a chemical, enzymatic, photolytic, or metabolic transformation within the body to release the active drug. Non-limiting examples of prodrug moieties include the hydrolytically sensitive or labile acyloxymethyl esters —CH₂C(═O)R′ and acyloxymethyl carbonates —CH₂C(═O)OR′ where R′ is C₁-C₆ alkyl, C₁-C₆ substituted alkyl, C₆-C₂₀ aryl, or C₆-C₂₀ substituted aryl, and amides.

As used herein, the term “alkyl” or “alkyl group” means a linear- or branched-chain saturated aliphatic hydrocarbon monovalent group, which may be unsubstituted or substituted. The group may be partially or completely substituted with halogen atoms (F, Cl, Br, or I). Non-limiting examples of alkyl groups include methyl, ethyl, n-propyl, 1-methylethyl(isopropyl), n-butyl, n-pentyl, 1,1-dimethylethyl (t-butyl), and the like. It may be abbreviated as “Alk”.

As used herein, the term “alkenyl” or “alkenyl group” means a linear- or branched-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

As used herein, the term “alkynyl” or “alkynyl group” means a linear- or branched-chain aliphatic hydrocarbon monovalent radical containing at least one carbon-carbon triple bond. This teen is exemplified by groups such as ethynyl, propynyl, n-butynyl, 2-butynyl, 3-methylbutynyl, n-pentynyl, heptynyl, octynyl, decynyl, and the like.

As used herein, the term “alkylene” or “alkylene group” means a linear- or branched-chain saturated aliphatic hydrocarbon divalent radical having the specified number of carbon atoms. This term is exemplified by groups such as methylene, ethylene, propylene, n-butylene, and the like, and may alternatively and equivalently be denoted herein as -(alkyl)-.

The term “alkenylene” or “alkenylene group” means a linear- or branched-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as -(alkylenyl)-.

The term “alkynylene” or “alkynylene group” means a linear- or branched-chain aliphatic hydrocarbon divalent radical containing at least one carbon-carbon triple bond. This term is exemplified by groups such as ethynylene, propynylene, n-butynylene, 2-butynylene, 3-methylbutynylene, n-pentynylene, heptynylene, octynylene, decynylene, and the like, and may alternatively and equivalently be denoted herein as -(alkynyl)-.

As used herein, the term “aryl” or “aryl group” means an aromatic monovalent or divalent radical of from 5 to 14 carbon atoms having a single ring (e.g., phenyl or phenylene), multiple condensed rings (e.g., naphthyl or anthranyl), or multiple bridged rings (e.g., biphenyl). In general, the term “aryl” or “aryl group” also includes “heteroaryl” or “heteroaryl group,” which is defined hereinafter. Unless otherwise specified, the aryl ring may be attached at any suitable carbon atom which results in a stable structure and, if substituted, may be substituted at any suitable carbon atom which results in a stable structure. Non-limiting examples of aryl groups include phenyl, naphthyl, anthryl, phenanthryl, indanyl, indenyl, biphenyl, and the like. It may be abbreviated as “Ar”.

The term “heteroaryl” or “heteroaryl group” means a stable aromatic 5- to 14-membered, monocyclic or polycyclic monovalent or divalent radical, which may comprise one or more fused or bridged ring(s), preferably a 5- to 7-membered monocyclic or 7- to 10-membered bicyclic radical, having from one to four heteroatoms in the ring(s) independently selected from nitrogen, oxygen, and sulfur, wherein any sulfur heteroatoms may optionally be oxidized and any nitrogen heteroatom may optionally be oxidized or be quaternized. One of the fused or bridge rings may be a non-aromatic ring. Unless otherwise specified, the heteroaryl ring may be attached at any suitable heteroatom or carbon atom which results in a stable structure and, if substituted, may be substituted at any suitable heteroatom or carbon atom which results in a stable structure. Non-limiting examples of heteroaryls include furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, benzoxazinyl, benzoxazinonyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl, and phenoxazinyl, and the like.

The term “heterocycle”, “heterocycle group”, “heterocyclyl”, “heterocyclyl group”, “heterocyclic”, or “heterocyclic group” means a stable non-aromatic 5- to 14-membered monocyclic or polycyclic, monovalent or divalent, ring which may comprise one or more fused or bridged ring(s), preferably a 5- to 7-membered monocyclic or 7- to 10-membered bicyclic ring, having from one to three heteroatoms in at least one ring independently selected from nitrogen, oxygen, and sulfur, wherein any sulfur heteroatoms may optionally be oxidized and any nitrogen heteroatom may optionally be oxidized or be quaternized. As used herein, the term “heterocycle” (or its equivalents) includes heterocycloalkyl, heterocycloalkenyl, and heterocycloalkynyl groups. Unless otherwise specified, the heterocyclyl ring may be attached at any suitable heteroatom or carbon atom which results in a stable structure and, if substituted, may be substituted at any suitable heteroatom or carbon atom which results in a stable structure. Non-limiting examples of heterocycles include pyrrolinyl, pyrrolidinyl, pyrazolinyl, pyrazolidinyl, piperidinyl, morpholinyl, thiomorpholinyl, piperazinyl, tetrahydropyranyl, tetrahydrothiopyranyl, tetrahydrofuranyl, hexahydropyrimidinyl, hexahydropyridazinyl, and the like.

The term “cycloalkyl” or “cycloalkyl group” means a stable aliphatic saturated 3- to 15-membered monocyclic or polycyclic monovalent radical consisting solely of carbon and hydrogen atoms which may comprise one or more fused or bridged ring(s), preferably a 5- to 7-membered monocyclic or 7- to 10-membered bicyclic ring. Unless otherwise specified, the cycloalkyl ring may be attached at any carbon atom which results in a stable structure and, if substituted, may be substituted at any suitable carbon atom which results in a stable structure. Exemplary cycloalkyl groups include cyclopropyl, cyclobutyl, cyclopentyl, cyclohexyl, cycloheptyl, cyclooctyl, cyclononyl, cyclodecyl, norbornyl, adamantyl, tetrahydronaphthyl (tetralin), 1-decalinyl, bicyclo[2.2.2]octanyl, 1-methylcyclopropyl, 2-methylcyclopentyl, 2-methylcyclooctyl, and the like.

The term “cycloalkenyl” or “cycloalkenyl group” means a stable aliphatic 5- to 15-membered monocyclic or polycyclic monovalent radical having at least one carbon-carbon double bond and consisting solely of carbon and hydrogen atoms which may comprise one or more fused or bridged ring(s), preferably a 5- to 7-membered monocyclic or 7- to 10-membered bicyclic ring. Unless otherwise specified, the cycloalkenyl ring may be attached at any carbon atom which results in a stable structure and, if substituted, may be substituted at any suitable carbon atom which results in a stable structure. Exemplary cycloalkenyl groups include cyclopentenyl, cyclohexenyl, cycloheptenyl, cyclooctenyl, cyclononenyl, cyclodecenyl, norbornenyl, 2-methylcyclopentenyl, 2-methylcyclooctenyl, and the like.

The term “cycloalkynyl” or “cycloalkynyl group” means a stable aliphatic 8- to 15-membered monocyclic or polycyclic monovalent radical having at least one carbon-carbon triple bond and consisting solely of carbon and hydrogen atoms which may comprise one or more fused or bridged ring(s), preferably a 8- to 10-membered monocyclic or 12- to 15-membered bicyclic ring. Unless otherwise specified, the cycloalkynyl ring may be attached at any carbon atom which results in a stable structure and, if substituted, may be substituted at any suitable carbon atom which results in a stable structure. Exemplary cycloalkynyl groups include cyclooctynyl, cyclononynyl, cyclodecynyl, 2-methylcyclooctynyl, and the like.

The term “carbocycle” or “carbocyclic group” means a stable aliphatic 3- to 15-membered monocyclic or polycyclic monovalent or divalent radical consisting solely of carbon and hydrogen atoms which may comprise one or more fused or bridged rings, preferably a 5- to 7-membered monocyclic or 7- to 10-membered bicyclic ring. Unless otherwise specified, the carbocycle may be attached at any carbon atom which results in a stable structure and, if substituted, may be substituted at any suitable carbon atom which results in a stable structure. The term comprises cycloalkyl (including spiro cycloalkyl), cycloalkylene, cycloalkenyl, cycloalkenylene, cycloalkynyl, and cycloalkynylene, and the like.

The terms “heterocycloalkyl”, “heterocycloalkenyl”, and “heterocycloalkynyl” mean cycloalkyl, cycloalkenyl, and cycloalkynyl group, respectively, having at least a heteroatom in at least one ring, respectively.

In general, the present invention provides pharmaceutical compounds and compositions for treating, reducing, ameliorating, or inhibiting the progression of, pathological ocular neovascularization in a subject.

In one aspect, the present invention also provides a method for treating, reducing, ameliorating, or inhibiting the progression of, pathological ocular neovascularization in a subject by using such compounds or compositions.

In another aspect, the present invention provides pharmaceutical compounds and compositions for treating, reducing, ameliorating, or inhibiting the progression of, a pathological ocular condition or disorder resulting from, or having an etiology in, ocular neovascularization in a subject.

In still another aspect, a compound or composition of the present invention causes a lower level of at least an adverse side effect than at least a prior-art glucocorticoid used to treat, reduce, or ameliorate the same condition or disorder.

In yet another aspect, such a condition or disorder is selected from the group consisting of diabetic retinopathy (“DR”), age-related macular degeneration (“AMD,” including dry and wet AMD), diabetic macular edema (“DME”), retinal detachment, posterior uveitis, corneal neovascularization, iris neovascularization, and combinations thereof.

In a further aspect, a pharmaceutical compound or a composition of the present invention comprises an integrin antagonist. In one embodiment, a pharmaceutical compound or a composition of the present invention comprises a vitronectin receptor antagonist.

In yet another aspect, the present invention provides a pharmaceutical compound or a composition for treating, reducing, ameliorating, or inhibiting the progression of, pathological ocular neovascularization in a subject, wherein the compound or composition comprises an integrin antagonist or a vitronectin receptor antagonist, wherein said integrin antagonist or a vitronectin receptor antagonist comprises a compound having Formula I

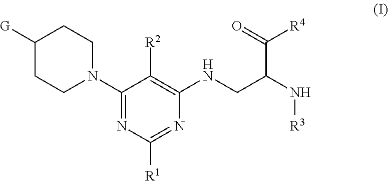

wherein G represents a substituted or unsubstituted aryl or heteroaryl group; substituted or unsubstituted heterocycle group; R⁷R⁸N—C(═NR⁶)—NH—CO-A-NH—CO—; A-NH—CH₂—; wherein A represents an aryl, heteroaryl, or heterocycle group, unsubstituted or substituted with one or more R⁹ groups;
R¹ represents a hydrogen atom; a halogen atom, a nitro group; (C₁-C₄)alkyl-; (C₁-C₄)alkoxy-; (C₅-C₁₄)Ar—; (C₅-C₁₄)Ar—(C₁-C₄)alkyl- group; an amino radical unsubstituted or monosubstituted or disubstituted with an alkyl radical and/or an acyl radical containing 1 to 4 carbon atoms, a —(CH₂)₀₋₂—C(O)OR⁵ group; or a —(CH₂)₀₋₂—OR⁵ group;
R² represents a hydrogen atom; a halogen atom; a nitro group; an alkyl radical containing 1 to 4 carbon atoms; a (C₁-C₄)alkoxy- group; an amino radical unsubstituted or monosubstituted or disubstituted with an alkyl and/or an acyl containing 1 to 4 carbon atoms; a —(CH₂)₀₋₂—C(O)OR⁵ group; or a —(CH₂)₀₋₂—OR⁵ group;
R³ represents a hydrogen atom, a —C(O)OR⁵ radical, an —SO₂R⁵ radical, or a monocyclic or polycyclic system comprising a 4- to 10-membered aromatic or non-aromatic ring system, the ring or at least one of the rings containing 1 to 4 heteroatoms chosen from N, O or S, unsubstituted or substituted with one or more R⁹ radicals;
R⁴ represents OH; (C₁-C₈)-alkoxy-; (C₅-C₁₄)—Ar—(C₁-C₄)-alkoxy-; (C₅-C₁₄)-aryloxy-; (C₃-C₁₂)-cycloalkyloxy; (C₃-C₁₂)-cycloalkyl-(C₁-C₄)-alkyloxy-; (C₁-C₈)-alkyl-carbonyloxy-(C₁-C₄)-alkyloxy-; (C₅-C₁₄)—Ar—(C₁-C₄)-alkyl-carbonyloxy-(C₁-C₄)-alkyloxy-; (C₁-C₈)dialkylaminocarbonylmethyloxy-; (C₅-C₁₄)—Ar—(C₁-C₄)-dialkylaminocarbonylmethyloxy-; an amino radical unsubstituted or monosubstituted or disubstituted with a (C₁-C₄)-alkyl and/or (C₅-C₁₄)—Ar and/or (C₅-C₁₄)—Ar—(C₁-C₄)-alkyl-radical and/or a (C₁-C₅)-acyl radical ; or the remainder of aspartic acid (D) or leucine (L);
R⁵ represents (C₁-C₈)-alkyl-; (C₁-C₈)-alkyl-C(O)O—(C₁-C₈)-alkyl-; (C₁-C₈)-alkyl-S(O)(O)—(C₁-C₈)-alkyl-; (C₅-C₁₄)—Ar—; (C₅-C₁₄)—Ar—(C₁-C₄)-alkyl-; (C₅-C₁₄)—Ar—C(O)O—(C₁-C₄)-alkyl-; (C₃-C₁₂)-cycloalkyl-; (C₃-C₁₂)-cycloalkyl-(C₁-C₄)-alkyl-; bicycloalkyl-(C₁-C₄)-alkyl-; tricycloalkyl-(C₁-C₄)-alkyl-; said Ar, alkyl, cycloalkyl, bicycloalkyl and tricycloalkyl radicals being unsubstituted or substituted by one or more R⁹ groups;
R⁶ represents a hydrogen atom; a hydroxyl; nitro; (C₁-C₆)-alkyl-O—C(O)—; (C₁-C₆)-alkyl-O—C(O)O— group;
R⁷ and R⁸, independently of one another represent a hydrogen atom or a (C₁-C₆)-alkyl radical unsubstituted or substituted with R⁹;
R⁹ represents halogen; amino; nitro; hydroxyl; (C₁-C₄)alkoxy; (C₁-C₄)alkylthio; carboxy; (C₁-C₄)alkyloxycarbonyl; (C₁-C₈)alkyl unsubstituted or substituted with one or more halogen atoms; (C₅-C₁₄)Ar; (C₅-C₁₄)Ar—(C₁-C₄)alkyl;
or one or more isomers of a compound having Formula I, alone or in a mixture, a pharmaceutically acceptable salt (such as addition salts), ester, hydrate, solvate, clathrate, or polymorph thereof.

The optically active carbon atoms contained in the compounds of Formula I can independently from one another show the R configuration or the S configuration.

The compounds of Formula I can be in the form of pure (or substantially pure; e.g., greater than 70, 80, 90, 95, or 99% enantiomeric excess) enantiomers or pure (or substantially pure; e.g., greater than 70, 80, 90, 95, or 99% diasteoisomeric excess) diastereoisomers, or in the form of a mixture of enantiomers, for example in the form of racemates or mixtures of diastereoisomers.

An aspect of the present invention is, therefore, pure (or substantially pure, as defined above) enantiomers, mixtures of these enantiomers, pure (or substantially pure, as defined above) diastereoisomers and mixtures of these diastereoisomers.

The invention relates to mixtures of two or more than two stereoisomers of Formula I and all the ratios of these stereoisomers in said mixtures.

The compounds of Formula I can, if appropriate, be present in the form of E isomers or Z isomers. An aspect of the invention is, therefore, the pure E isomers, the pure Z isomers and the E/Z mixtures in any ratio.

The invention also relates to all the tautomer forms of the compounds of Formula I, relating for example to the form represented by Formula I, with G being (R⁷)(R⁸)N—C(═NR⁶)—NH—CO—, the form in which acylguanidine is present in the form of a —CO—N═C(NHR¹)—NR²R⁷ group, and all the other forms, which differ by the different position of the hydrogen atom.

The diastereoisomers, including the E/Z isomers, can be separated into individual isomers, for example by chromatography. The racemates can be separated into two enantiomers by standard methods such as chiral phase chromatography or by resolution methods.

The pharmaceutically acceptable salts of the compounds of Formula I are, in particular, salts which can be used pharmaceutically or non-toxic salts or salts which can be used physiologically.

When the compounds of Formula I contain an acid group such as carboxylic acid, they are for example salts of alkali or alkaline earth metals such as sodium, potassium, magnesium, calcium salts, and also the salts formed with pharmaceutically acceptable quaternary ammonium ions and the addition salts with acids such as ammonia and pharmaceutically acceptable organic amines such as for example triethylamine, ethanolamine or tris-(2-hydroxyethyl)amine.

When the compounds of Formula I contain a basic group, they can form an addition salt with acids, for example with inorganic acids such as hydrochloric, sulphuric, phosphoric acid or with organic carboxylic acids such as acetic, trifluoroacetic, citric, benzoic, maleic, fumaric, tartaric, methanesulphonic or para toluene sulphonic acid.

The compounds of Formula I which comprise a basic group and an acid group, such as for example guanidino and carboxylic, can be present in the form of Zwitterions (betaines), which are also included in the present invention.

The salts of the compounds of Formula I can be obtained by standard methods known to a person skilled in the art, for example by combining a compound of Formula I with an organic or inorganic acid or a base in a solvent or a dispersant or from another salt by cation or anion exchange.

The invention also includes all the salts of the compounds of Formula I which, because of their low physiological acceptability, cannot be used directly as medicaments, but can be used as intermediate products to implement subsequent chemical modifications in the compounds of Formula I or as starting products for the preparation of pharmaceutically acceptable salts.

The present invention also includes all the solvates of the compounds of Formula I for example the hydrates, the solvates formed with alcohols, and all the derivatives of the compounds of Formula I, for example the esters, prodrugs and other physiologically acceptable derivatives, as well as the metabolites of the compounds of Formula I.

In yet another aspect, the present invention provides a pharmaceutical compound or a composition for treating, reducing, ameliorating, or inhibiting the progression of, pathological ocular neovascularization in a subject, wherein the compound or composition comprises an integrin antagonist or a vitronectin receptor antagonist, wherein said integrin antagonist or a vitronectin receptor antagonist comprises a compound having Formula I

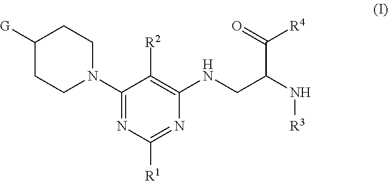

wherein G represents a substituted or unsubstituted aryl or heteroaryl group; substituted or unsubstituted heterocycle group;
R¹ represents a hydrogen atom; a (C₁-C₃)alkyl- group; or a (C₁-C₃)alkoxy- group;
R² represents a hydrogen atom; a halogen atom; a nitro group; a (C₁-C₃)alkyl- group; a (C₁-C₃)alkoxy- group; or an amino radical unsubstituted or monosubstituted or disubstituted with an alkyl and/or an acyl containing 1 to 4 carbon atoms;
R³ represents a hydrogen atom; a —C(O)OR^{S} radical; or an —S(O)(O)R⁵ radical;
R⁴ represents OH; (C₁-C₃)alkoxy-; or (C₅-C₁₀)aryloxy-;
R⁵ represents (C₁-C₃)alkyl-; (C₅-C₁₀)Ar—; (C₅-C₁₀)heteroaryl-; (C₅-C₁₀)heterocycle-; said Ar, heteroaryl, alkyl, heterocycle radicals being unsubstituted or substituted by one or more R⁹ groups; and
R⁹ represents halogen; amino; nitro ;hydroxyl; (C₁-C₃)alkyl-; or (C₁-C₃)alkoxy-;
or one or more isomers of a compound having Formula I, alone or in a mixture, a pharmaceutically acceptable salt (such as addition salts), ester, hydrate, solvate, or clathrates thereof.

In one aspect, the group G of the compounds of Formula I represents A, A-NHC(O)—, or A-NH—CH₂— group in which A represents

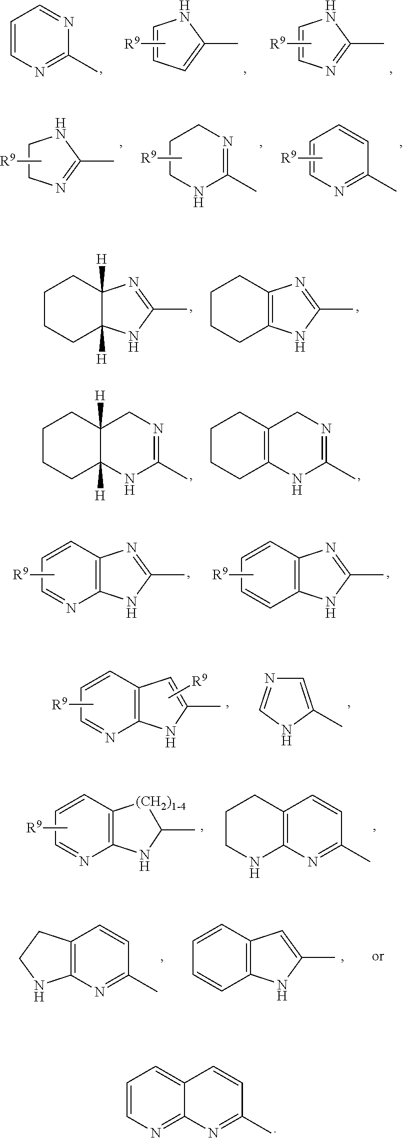

In another aspect, the group A is selected from the group consisting of

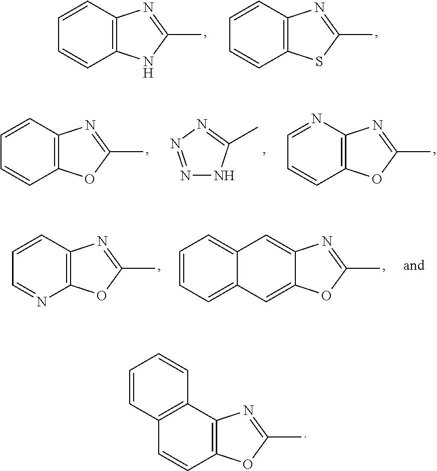

In another aspect, the group A is selected from the group consisting of

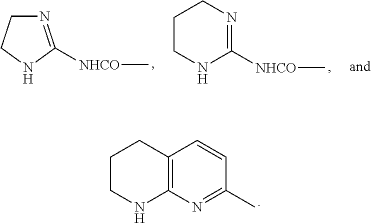

In still another aspect, the group A is selected from the group consisting of

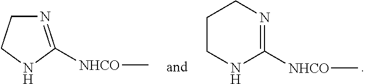

In still another aspect, the group A comprises, or consists of

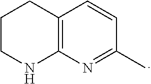

An integrin or vitronectin receptor antagonist of the present invention, having Formula I, has an αᵥβ₃ binding IC₅₀ of about 50 μM or less; preferably, 15 μM or less; more preferably, 10 μM or less; or even more preferably, 5 μM or less.

In some embodiments, the integrin or vitronectin receptor antagonist of, or used in, the present invention is selected from the group consisting of the compounds having Formulae II-X, free acid forms thereof, and pharmaceutical acceptable salts, esters, hydrates, solvates, clathrates, and polymorphs thereof.

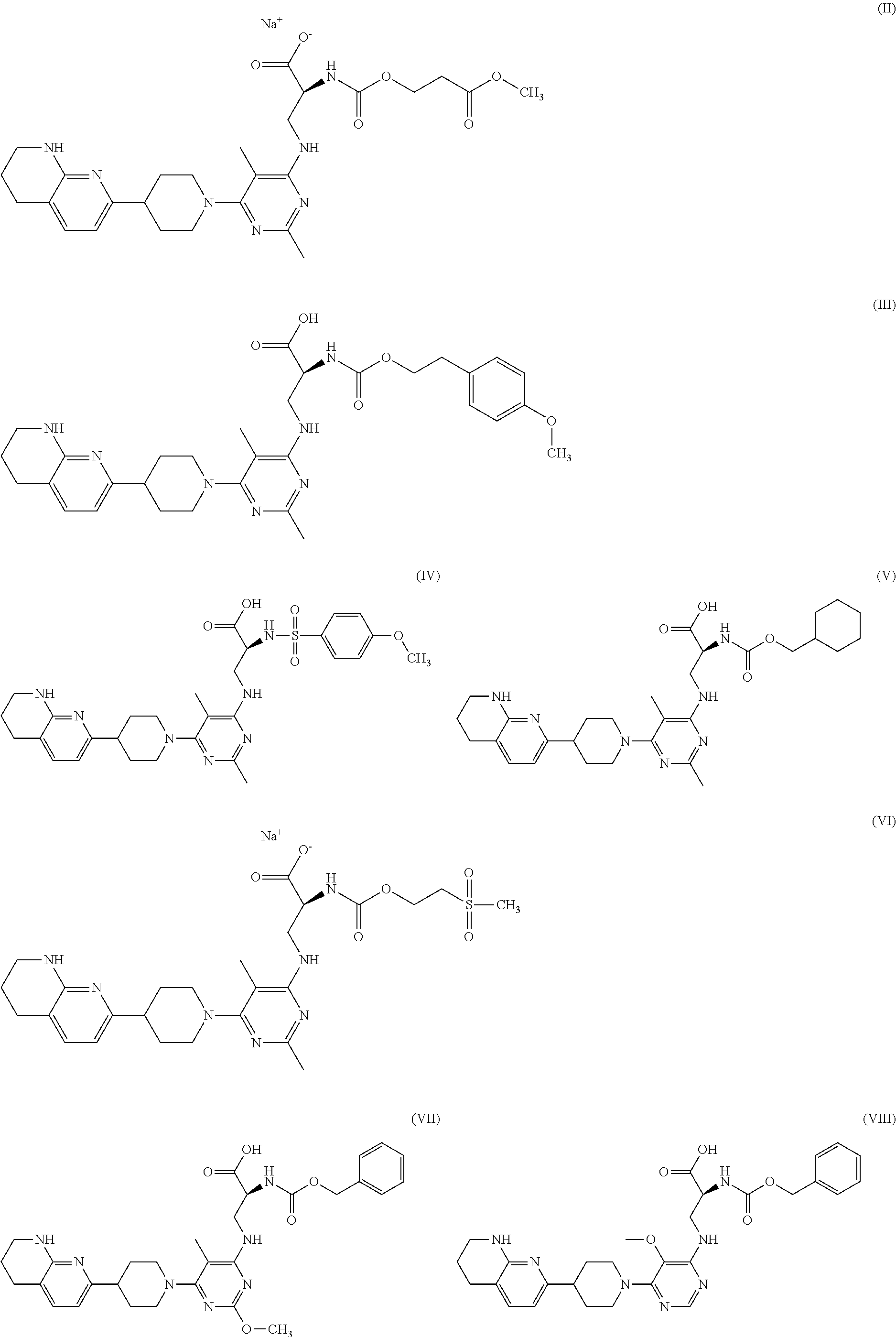

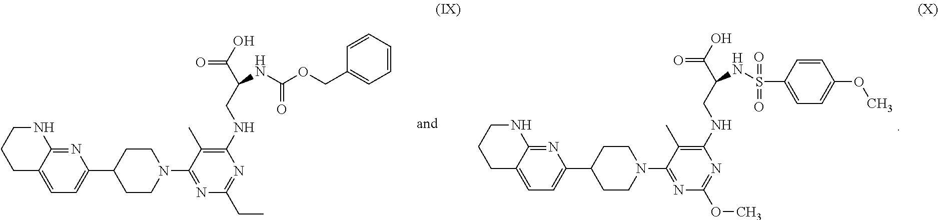

The compounds having Formulae II-X are also referred to hereinafter as BOL-303048-S, BOL-303049-X, BOL-303050-X, BOL-303051-X, BOL-303052-S, BOL-303053-X, BOL-303054-X, BOL-303055-X, and BOL-303056-X, respectively.

In one embodiment, a preferred integrin or vitronectin receptor antagonist of, or used in, the present invention comprises or consists of the compound having Formula Iv.

In another embodiment, a preferred integrin or vitronectin receptor antagonist of, or used in, the present invention comprises or consists of a compound having Formula XI,

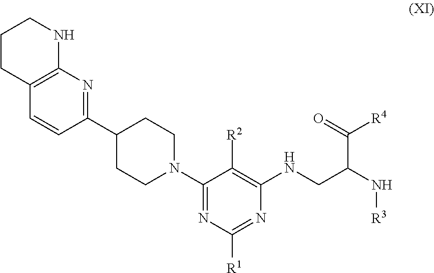

wherein R¹, R², R³, and R⁴ are defined herein above.

In yet another embodiment, a preferred integrin or vitronectin receptor antagonist of, or used in, the present invention comprises or consists of a compound having Formula XI, wherein R¹ and R² are independently selected from the group consisting of (C₁-C₃)alkyl and (C₁-C₃)alkoxy; R³ is selected from the group consisting of —C(O)O—(C₁-C₃)alkyl-C(O)O—(C₁-C₃)alkyl-, —C(O)O—(C₁-C₃)alkyl-Ar—, —C(O)O—(C₁-C₃)alkyl-(C₃-C₆)cycloalkyl-, —S(O)(O)—Ar, —S(O)(O)—(C₁-C₃)alkyl-Ar—, and —C(O)O—(C₁-C₃)alkyl-S(O)(O)—(C₁-C₃)alkyl, wherein Ar may be substituted with one or more (C₁-C₃)alkyl or (C₁-C₃)alkoxy groups.

The compounds having Formulae I-XI can be synthesized according a method that is disclosed in U.S. Pat. No. 7,582,640, or US Patent Application Publication 2008/0058348, which are incorporated herein by reference in their entireties.

In another aspect, the present invention provides a method for treating, reducing, ameliorating, or inhibiting pathological ocular neovascularization in a subject, the method comprising administering to an ocular environment of an affected eye of said subject a therapeutically effective amount of a composition that comprises at least a compound selected from compounds having Formulae I-XI, pharmaceutically acceptable salts, esters, hydrates, solvates, clathrates, and polymorphs thereof, and combinations thereof, at a frequency that results in an effective treatment, reduction, amelioration, or inhibition of said pathological ocular neovascularization.

In still another aspect, the present invention provides a method for treating, reducing, ameliorating, or inhibiting pathological ocular neovascularization in a subject, the method comprising administering to an ocular environment of an affected eye of said subject a therapeutically effective amount of a compound selected from the group consisting of the compounds having Formulae II-IX, pharmaceutically acceptable salts, esters, hydrates, solvates, clathrates, and polymorphs thereof, and combinations thereof, at a frequency that results in an effective treatment, reduction, amelioration, or inhibition of said pathological ocular neovascularization.

In another aspect, the present invention provides a method for treating, reducing, ameliorating, or inhibiting pathological ocular neovascularization in a subject, the method comprising administering to an ocular environment of an affected eye of said subject a therapeutically effective amount of the compound having Formula IV, a pharmaceutically acceptable salt, ester, hydrate, solvate, clathrate, or polymorph thereof, at a frequency that results in an effective treatment, reduction, amelioration, or inhibition of said pathological ocular neovascularization. Said frequency can be determined by a skilled medical practitioner from assessment of the condition of a particular patient.

In another aspect, the composition is administered topically to the affected eye, by injection to or into the conjunctiva, anterior segment, posterior segment, or vitreous of the affected eye, or by periocular injection to the affected eye.

In still another aspect, the composition is administered to said subject at a dose that provides an amount of the integrin or vitronectin receptor antagonist from about 0.0001 mg to about 1 mg (or alternatively, from about 0.001 mg to about 0.5 mg, or from about 0.001 mg to about 0.2 mg, or from about 0.001 mg to about 0.1 mg, or from about 0.01 mg to about 0.1 mg, or from about 0.01 mg to about 0.05 mg, or from about 0.001 mg to about 0.01 mg, or from about 0.001 mg to about 0.05 mg) per kg body weight of the subject.

In yet another aspect, a therapeutically effective amount of the composition is administered topically to the affected eye once, twice, three, or four times, per day.

In a further aspect, a therapeutically effective amount of the composition is injected to or into an environment (such as the vitreous or choroid) of the affected eye once every one, two, three, four, or six months, or once per year. An amount suitable for such treatment may be selected within the range disclosed above by a skilled medical practitioner. Alternatively, a low dose such as 0.0001 mg/kg of body weight initially may be attempted, then a subsequent dose may be adjusted based on the result of the previously administered dose.

In another aspect of the present invention, a compound or composition disclosed herein causes a lower level of at least an adverse side effect than at least a prior-art glucocorticoid used to treat, reduce, ameliorate, or inhibit the same condition or disorder. In one aspect, such a condition or disorder results from pathological neovascularization. In another aspect, such pathological neovascularization has an etiology in chronic inflammation, for example, as a result of diabetes mellitus, or chronic inflammation of the ocular vasculature.

In one aspect, a level of said at least an adverse side effect is determined in vivo or in vitro. For example, a level of said at least an adverse side effect is determined in vitro by performing a cell culture and determining the level of a biomarker associated with said side effect. Such biomarkers can include proteins (e.g., enzymes), lipids, sugars, and derivatives thereof that participate in, or are the products of, the biochemical cascade resulting in the adverse side effect. Representative in vitro testing methods are further disclosed hereinbelow.

In another aspect, said at least an adverse side effect is selected from the group consisting of glaucoma, cataract, hypertension, hyperglycemia, hyperlipidemia (increased levels of triglycerides), and hypercholesterolemia (increased levels of cholesterol). A side effect such as hypertension, hyperglycemia, hyperlipidemia, or hypercholesterolemia can be a systemic side effect. In one embodiment, a level of said at least an adverse side effect is determined at about one day after said compounds or compositions are first administered to, and are present in, said subject. In another embodiment, a level of said at least an adverse side effect is determined about 14 days after said compounds or compositions are first administered to, and are present in, said subject. In still another embodiment, a level of said at least an adverse side effect is determined about 30 days after said compounds or compositions are first administered to, and are present in, said subject. Alternatively, a level of said at least an adverse side effect is determined about 2, 3, 4, 5, or 6 months after said compounds or compositions are first administered to, and are present in, said subject.

In another aspect, said at least a prior-art glucocorticoid used to treat, reduce, ameliorate, or alleviate the same condition or disorder is administered to said subject at a dose and a frequency sufficient to produce an equivalent beneficial effect on said condition or disorder as a compound or composition of the present invention after about the same elapsed time.

In still another aspect, said at least a prior-art glucocorticoid is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, combinations thereof, and mixtures thereof. In one embodiment, said at least a prior-art glucocorticoid is selected from the group consisting of dexamethasone, prednisone, prednisolone, methylprednisolone, medrysone, triamcinolone, loteprednol etabonate, physiologically acceptable salts thereof, combinations thereof, and mixtures thereof. In another embodiment, said at least a prior-art glucocorticoid is acceptable for ophthalmic uses.

In another aspect, the present invention provides an ophthalmic pharmaceutical composition for treating, reducing, ameliorating, alleviating, or inhibiting pathological ocular neovascularization. In one embodiment, such pathological ocular neovascularization is neovascularization of the retina, resulting in, diabetic retinopathy or diabetic macular edema. In another embodiment, such pathological ocular neovascularization is neovascularization of the choroid, resulting in macular degeneration. In still another embodiment, such pathological neovascularization is the result of chronic inflammation of the ocular vasculature. The ophthalmic pharmaceutical composition comprises at least an integrin antagonist or vitronectin receptor antagonist selected from the compounds having Formulae I-XI, a prodrug thereof, or a pharmaceutically acceptable salt, ester, hydrate, solvate, clathrate, or polymorph thereof. In one aspect, the pharmaceutical composition further comprises a pharmaceutically acceptable carrier.

The concentration of an integrin antagonist or vitronectin receptor antagonist disclosed herein, a prodrug thereof, or a pharmaceutically acceptable salt, ester, hydrate, solvate, or clathrate thereof in such an ophthalmic composition can be in the range from about 0.0001 to about 200 mg/g of the composition (or, alternatively, from about 0.001 to about 200 mg/g, or from about 0.001 to about 150 mg/g, or from about 0.001 to about 100 mg/g, or from about 0.001 to about 80 mg/g, or from about 0.001 to about 50 mg/g, or from about 0.01 to about 200 mg/g, or from about 0.01 to about 150 mg/g, or from about 0.01 to about 100 mg/g, or from about 0.1 to about 100 mg/g, or from about 0.1 to about 50 mg/g, or from about 0.1 to about 25 mg/g of the composition).

In one embodiment, a composition of the present invention is in a form of a suspension or dispersion. In another embodiment, the suspension or dispersion is based on an aqueous medium. For example, a composition of the present invention can comprise sterile saline solution. In still another embodiment, micrometer- or nanometer-sized particles of a compound having Formulae I-XI, or prodrug thereof, or a pharmaceutically acceptable salt, ester, hydrate, solvate, clathrate, or polymorph thereof can be coated with a physiologically acceptable surfactant (non-limiting examples are disclosed below), then the coated particles are dispersed in an liquid medium. The coating can keep the particles in a suspension.

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1, or from about 0.01 to about 0.5 weight percent).

In addition, a composition of the present invention can include additives such as buffers, diluents, carriers, adjuvants, anti-oxidants, or other excipients. Any pharmacologically acceptable buffer suitable for application to the eye may be used. Other agents may be employed in the composition for a variety of purposes. For example, buffering agents, preservatives, co-solvents, oils, humectants, emollients, stabilizers, or antioxidants may be employed. Water-soluble preservatives which may be employed include sodium bisulfite, sodium bisulfate, sodium thiosulfate, benzalkonium chloride, chlorobutanol, thimerosal, ethyl alcohol, methylparaben, polyvinyl alcohol, benzyl alcohol, and phenylethyl alcohol. These agents may be present in individual amounts of from about 0.001 to about 5% by weight (preferably, about 0.01% to about 2% by weight). Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration. These agents may be present in amounts sufficient to maintain a pH of the system of between about 3 and about 10. As such, the buffering agent may be as much as about 5% on a weight to weight basis of the total composition. Electrolytes such as, but not limited to, sodium chloride and potassium chloride may also be included in the formulation.

In one aspect, the pH of the composition is in the range from about 4 to about 10. Alternatively, the pH of the composition is in the range from about 5 to about 9, from about 6 to about 9, or from about 6.5 to about 8. In another aspect, the composition comprises a buffer having a pH in one of said pH ranges.

In another aspect, the composition has a pH of about 7. Alternatively, the composition has a pH in a range from about 7 to about 7.5.

In still another aspect, the composition has a pH of about 7.4.

In yet another aspect, a composition also can comprise a viscosity-modifying compound designed to facilitate the administration of the composition into the subject or to promote the bioavailability in the subject. In still another aspect, the viscosity-modifying compound may be chosen so that the composition is not readily dispersed after being administered into the vistreous. Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cps” or mPa·s); preferably, from about 1 to about 100 cps, measured with a Brookfield viscometer.

In yet another aspect, the present invention provides a composition for treating, reducing, ameliorating, alleviating, or inhibiting the progression of, pathological ocular neovascularization. In one embodiment, the pathological ocular neovascularization has an etiology in inflammation. In another embodiment, such pathological ocular neovascularization is selected form the group consisting of diabetic retinopathy (“DR”), age-related macular degeneration (“AMD”, including exudative AMD), and diabetic macular edema (“DME”).

In one aspect, the composition comprises at least a compound selected from compounds having Formulae II-X, a prodrug thereof, or a pharmaceutically acceptable salt, ester, hydrate, solvate, clathrate, or polymorph thereof.

In another aspect, the composition comprises: (a) at least a compound selected from compounds having Formulae I-XI, a prodrug thereof, or a pharmaceutically acceptable salt, ester, hydrate, solvate, clathrate, or polymorph thereof; and (b) an anti-angiogenic agent. In one embodiment, the anti-angiogenic agent comprises an antibody or a fragment thereof that binds to VEGF-A. Non-limiting examples of such antibody or antibody fragment include bevacizumab and ranibizumab.

In still another aspect, the composition comprises: (a) at least a compound selected from compounds having Formulae I-XI, a prodrug thereof, or a pharmaceutically acceptable salt, ester, hydrate, solvate, clathrate, or polymorph thereof; and (b) a material selected from the group consisting of (i) anti-inflammatory agents; (ii) anti-angiogenic agents; and (iii) combinations thereof; said compound, prodrug thereof, or pharmaceutically acceptable salt, ester, hydrate, solvate, or clathrate thereof, anti-inflammatory agent, or anti-angiogenic agent being present in amounts effective to treat, reduce, ameliorate, alleviate, inhibit the progression of, said pathological ocular neovascularization. In one embodiment, such an anti-inflammatory agent is selected from the group consisting of non-steroidal anti-inflammatory drugs (“NSAIDs”), peroxisome proliferator-activated receptor-γ (“PPARγ”) ligands, combinations thereof, and mixtures thereof.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

In another aspect of the present invention, an anti-inflammatory agent is a PPAR-binding molecule. In one embodiment, such a PPAR-binding molecule is a PPARα-, PPARδ-, or PPARγ-binding molecule. In another embodiment, such a PPAR-binding molecule is a PPARα, PPARδ, or PPARγ agonist. Such a PPAR ligand binds to and activates PPAR to modulate the expression of genes containing the appropriate peroxisome proliferator response element in its promoter region.

PPARγ agonists can inhibit the production of TNF-α and other inflammatory cytokines by human macrophages (C-Y. Jiang et al., Nature, Vol. 391, 82-86 (1998)) and T lymphocytes (A. E. Giorgini et al., Horm. Metab. Res. Vol. 31, 1-4 (1999)). More recently, the natural PPARγ agonist 15-deoxy-Δ-12,14-prostaglandin J2 (or “15-deoxy-Δ-12,14-PG J2”), has been shown to inhibit neovascularization and angiogenesis (X. Xin et al., J. Biol. Chem. Vol. 274:9116-9121 (1999)) in the rat cornea. Spiegelman et al., in U.S. Pat. No. 6,242,196, disclose methods for inhibiting proliferation of PPARγ-responsive hyperproliferative cells by using PPARγ agonists; numerous synthetic PPARγ agonists are disclosed by Spiegelman et al., as well as methods for diagnosing PPARγ-responsive hyperproliferative cells. All documents referred to herein are incorporated by reference. PPARs are differentially expressed in diseased versus normal cells. PPARγ is expressed to different degrees in the various tissues of the eye, such as some layers of the retina and the cornea, the choriocapillaris, uveal tract, conjunctival epidermis, and intraocular muscles (see, e.g., U.S. Pat. No. 6,316,465).

In one aspect, a PPARγ agonist used in a composition or a method of the present invention is a thiazolidinedione, a derivative thereof, or an analog thereof. Non-limiting examples of thiazolidinedione-based PPARγ agonists include pioglitazone, troglitazone, ciglitazone, englitazone, rosiglitazone, and chemical derivatives thereof. Other PPARγ agonists include Clofibrate (ethyl 2-(4-chlorophenoxy)-2-methylpropionate), clofibric acid (2-(4-chlorophenoxy)-2-methylpropanoic acid), GW 1929 (N-(2-benzoylphenyl)-O-{2-(methyl-2-pyridinylamino)ethyl}-L-tyrosine), GW 7647 (2-{{4-{2-{{(cyclohexylamino)carbonyl}(4-cyclohexylbutyl)amino}ethyl}phenyl}thio}-2-methylpropanoic acid), and WY 14643 ({{4-chloro-6-{(2,3-dimethylphenyl)amino}-2-pyrimidinyl}thio}acetic acid). GW 1929, GW 7647, and WY 14643 are commercially available, for example, from Koma Biotechnology, Inc. (Seoul, Korea). In one embodiment, the PPARγ agonist is 15-deoxy-Δ-12, 14-PG J2.

Non-limiting examples of PPAR-α agonists include the fibrates, such as fenofibrate and gemfibrozil. A non-limiting example of PPAR-δ agonist is GW501516 (available from Axxora LLC, San Diego, Calif. or EMD Biosciences, Inc., San Diego, Calif.).

In a further aspect, an anti-angiogenic agent included in a pharmaceutical composition of the present invention is selected from the group consisting of: (i) compounds that interact with and inhibit a downstream activity of extracellular VEGF (in particular, VEGF-A); (ii) compounds that interact with at least a VEGF receptor (in particular, VEGF-A receptor) and render it substantially unavailable for interacting with VEGF (in particular, VEGF-A); (iii) compounds that reduce a level of expression of VEGF (in particular, VEGF-A); and (iv) combinations thereof. The agents selected from the groups (i), (ii), (iii), and (iv) are also referred to collectively as “VEGF inhibitors” (in particular, “VEGF-A inhibitor”). The term “VEGF-A,” as used herein, also includes one or more of the VEGF-A isoforms, produced in vivo by alternative splicing: VEGF₁₂₁, VEGF₁₆₅, VEGF₁₈₉, and VEGF₂₀₆.

In one embodiment, compounds that interact with and inhibit a downstream activity of extracellular VEGF (in particular, VEGF-A) comprise a nucleic acid ligand that binds to extracellular VEGF (in particular, VEGF-A) and substantially prevents it from participating in the angiogenic cascade. Non-limiting examples of such a nucleic acid ligand are the VEGF aptamers disclosed in U.S. Pat. Nos. 6,426,335; 6,168,778; 6,147,204; 6,051,698; and 6,011,020; which are incorporated herein by reference in their entireties. In one embodiment, such a nucleic acid ligand comprises the VEGF antagonist aptamer known by its trade name “Macugen®”, being marketed by OSI EyeTech Pharmaceuticals (Melleville, N.Y.). In another embodiment, a compound that interacts with and inhibits a downstream activity of extracellular VEGF (in particular, VEGF-A) comprises an anti-VEGF (in particular, anti-VEGF-A) antibody, such as the recombinant monoclonal antibody known as Lucentis® (ranibizumab) or Avastin® (bevacizumab), both developed by Genentech, South San Francisco, Calif., or a combination thereof.

In one aspect of the present invention, compounds that interact with at least a VEGF receptor and render it substantially unavailable for interacting with VEGF (in particular, VEGF-A) comprises VEGF (in particular, VEGF-A) tyrosine kinase inhibitors, which can be a small synthetic molecule or protein or protein fragment that binds to the transmembrane VEGF receptors and neutralizes their activation, such as rendering them incapable of initiating or participating further in the expression of VEGF (in particular, VEGF-A) or other angiogenic factors.

Non-limiting examples of synthetic VEGF (or in particular, VEGF-A) tyrosine kinase inhibitors include the compounds disclosed in U.S. Pat. Nos. 6,958,340; 6,514,971; 6,448,077; and U.S. Patent Application Publications 2005/0233921, 2005/0244475, 2005/0143442, and 2006/0014252; which are incorporated herein by reference in their entirety.

In another aspect, a level of VEGF (or in particular, VEGF-A) can be reduced by interfering with the transcription of the VEGF gene by binding a small organic VEGF gene (in particular, VEGF-A gene) inhibitor to said gene, such as one of the compounds disclosed in U.S. Patent Application Publication 2003/0282849, which is incorporated herein by reference.

Other suitable anti-angiogenic agents that can be used in a composition of the present invention are disclosed in U.S. Patent Application Ser. No. 60/797,608 filed May 7, 2006, which is incorporated herein by reference.

### Testing 1: Study Evaluating the Effect of Integrin Antagonists on Network Formation of Human Retinal Endothelial Cells in Fibrin Gel

### Purpose

To investigate the effect of integrin antagonists on network formation of HREC in fibrin gel.

### Materials and Methods

### Assay Design

Prior to the start of the experiment, HREC from Cell Systems were cultured in flask in CS-C complete medium. The tube formation assay was performed in a 96-well plate pre-coated with fibrin gel. HREC were collected from flasks and re-suspended 1% FBS EBM-2 medium. Cells were pre-incubated with the integrin antagonists, echistatin or vehicle control for 30 min before loading onto the fibrin gel. Images were taken at 9 h. (Applicants, in a separate study, have demonstrated that echistatin, a RGD-containing peptide, significantly inhibits PMA-induced tube formation of HREC in fibrin gel.) Cell network formation was determined by scoring a method as described in the assay kit instructions.

TABLE 1
Experimental design and schedules

| | | Day 2 | |
|---|---|---|---|
| | | The following solutions were | |
| | | prepared and mixed | |
| | | with cells for 30 min before | |
| Group* | Day 1 | loading onto fibrin gel | |
| 1 | Fibrin gel | Basal (0.1% DMSO) | Images were |
| 2 | was | PMA 25 ng/mL | taken and the |
| 3 | prepared | PMA + Echistatin 10 nM | network |
| 4 | in a 96- | PMA + BOL-303049-X, 1 nM | formation was |
| 5 | well plate | PMA + BOL-303049-X, 10 nM | determined by |
| 6 | O/N | PMA + BOL-303050-X, 1 nM | the scoring |
| 7 | | PMA + BOL-303050-X, 10 nM | system |
| 8 | | PMA + BOL-303051-X, 1 nM | |
| 9 | | PMA + BOL-303051-X, 10 nM | |
| 10 | | PMA + BOL-303054-X, 1 nM | |
| 11 | | PMA + BOL-303054-X, 10 nM | |
| 12 | | PMA + BOL-303055-X, 1 nM | |
| 13 | | PMA + BOL-303055-X, 10 nM | |
| 14 | | Basal (0.1% DMSO) | |
| 15 | | PMA 25 ng/mL | |
| 16 | | PMA + Echistatin 10 nM | |

*triplicate wells per group

Compound stock solutions were made up in DMSO and stored at −20° C. To limit DMSO concentration to 0.1%, for every final incubation concentration, a 1000 times working concentration was prepared in DMSO.

Human retinal endothelial cells (HREC) were purchased from Cell System (Kirkland, Wash.). The cells are routinely cultured and maintained in complete medium containing 10% FBS before experiment. Passages 3-6 were normally used.

### Other Reagents

| Reagent | Catalog # | Lot # | Vendor |
|---|---|---|---|
| Fibrin Gel In | ECM630 | PSO1405570 | Chemicon (Temecula, CA) |
| Vitro | | | |
| Angiogenesis | | | |
| Assay Kit | | | |
| Trypsin | 25200 | 434755 | Gibco (Carlsbad, CA) |
| Echistatin | E1518 | 127K4050 | Sigma (St. Louis, MO) |
| Primary human | ACBR1 181 | N/A | Cell System |
| retinal | | | (Kirkland, WA) |
| endothelial | | | |
| cells | | | |
| (HREC) | | | |
| PMA | P1585-1 mg | 087K1079 | Sigma (St. Louis, MO) |
| EBM-2 | CC-3156 | 0000085573 | Clonetics |
| | | | (Walkersville, MD) |
| ALBUMAX | 11020-021 | 492034 | Gibco (Carlsbad. CA) |

### Assay Procedures (Following the Assay Kit Instructions)

Thrombin solution and fibrinogen solutions were allowed to thaw at room temperature and 37° C. water bath, respectively. Fibrinogen solution at 30 μL was added into the desired number of wells in a 96-well plate. Plate was gently shaken to ensure uniform coverage of fibrinogen solution in the well. Thrombin at 20 μL was added into each well. The plate was gently shaken immediately to ensure the thrombin was mixed well with fibrinogen. The plate was placed at 37° C. overnight to allow the fibrin gel to polymerize.

HREC were trypsinized at about 80% confluence and counted by standard hemocytometer methods. After deactivating trypsin with 5 mL of 10% FBS EBM-2 medium, the cells were centrifuged (800 rpm, 8 min) and re-suspended at 2×10⁵ cells/mL in 1% FBS EBM-2 medium

Cells were pre-incubated with integrin compounds or echistatin at 37° C., 5% CO₂, and 95% humidity for 30 minutes before mixing with PMA medium. The final concentrations of all agents are shown in the design table. The 100 μL of the cell mixture was added onto the gel and cells were cultured at 37° C., 5% CO₂, and 95% humidity. Images were taken at different time points.

### Semi-Quantification

Cell network formation was determined by scoring method as described in the assay kit instructions and also shown in the Table below:

The network formation scoring system (a five point-scale)

| | |
|---|---|
| Cells isolated or in a sheet-like monolayer | 0 |
| Cells begin to migrate and align themselves | 1 |
| Capillary tubes visible. No sprouting. | 2 |
| Sprouting of new capillary tubes visible. | 3 |
| Closed polygons begin to form. | 4 |
| Complex mesh like structures develop | 5 |

### Data Analysis

Each well in one group was given a score and then averaged. Data were expressed as mean±SEM of network formation scores. Statistical analysis was performed using a one-way ANOVA-Dunnett's test in raw data, and p<0.05 was considered statistically significant. Data analysis was performed using statistical analysis software JMP (SAS Institute, Cary, N.C.).

### Results

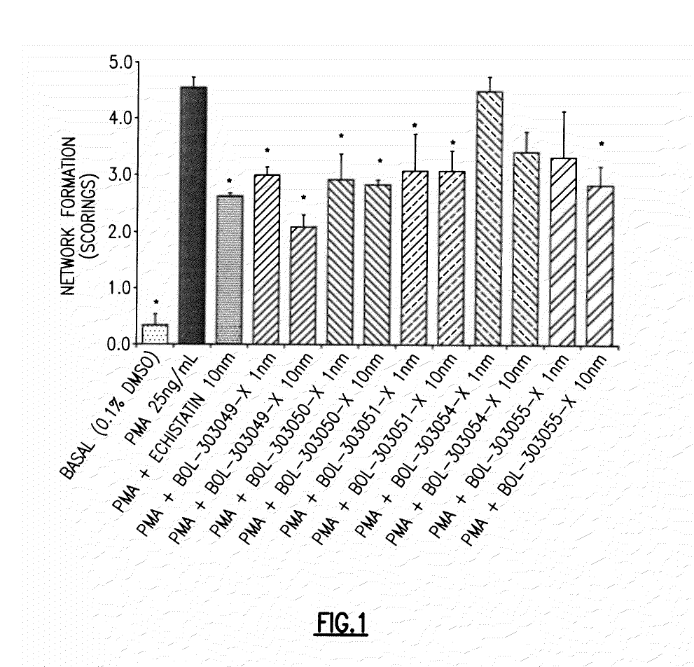

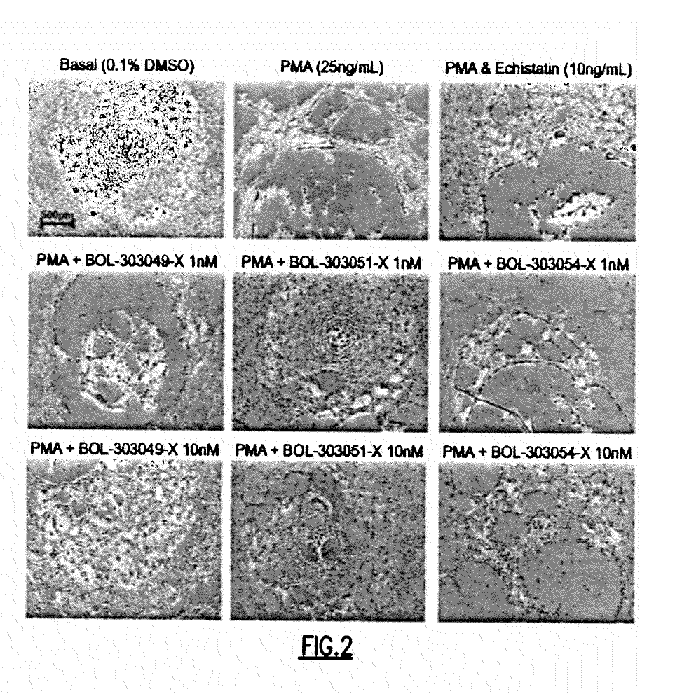

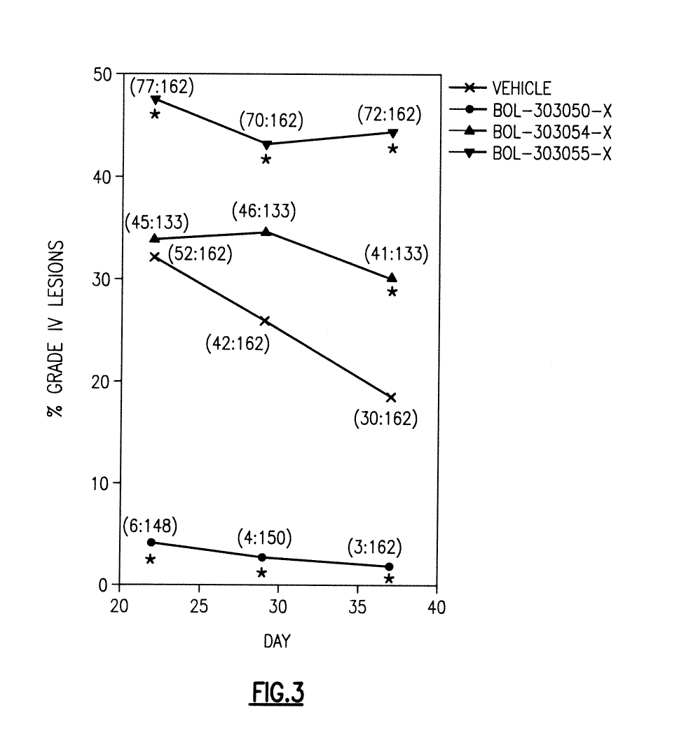

Under basal conditions, the network formation of HREC in fibrin gel was minimal. Addition of PMA at 25 ng/mL into the medium significantly induced network formation of HREC in figrin gel (FIGS. 1 and 2).

The RGD-containing peptide Echistatin at 10 nM significantly inhibited network formation of HREC in fibrin gel (FIGS. 1 and 2).

BOL-303049-X, BOL-303050-X and BOL-303051-X at 1 and 10 nM, and BOL-303055-X at 10 nM significantly inhibited HREC network formation. The inhibiting effect of BOL-303054-X at 1 and 10 nM on HREC network formation was not statistically significant in this assay (FIGS. 1 and 2).

### Conclusion

The integrin antagonists BOL-303049-X, BOL-303050-X, BOL-303051-X and BOL-303055-X show significant inhibition of PMA-induced HREC network formation at the doses of <10 nM. However, a higher dose may be needed for BOL-303054-X in order to reach a significant inhibiting effect.

### Testing 2: Effect of Integrin Receptor Antagonists on Laser-Induced Choroidal Neovascularization

### Purpose:

To evaluate the efficacy of four integrin antagonists in the laser-induced CNV in monkeys.

### Materials and Methods:

### Experimental Design

Study was performed in three phases. In phases Ia and Ib, the behavior of the suspension in aqueous (Ia) and oil (Ib) formulations, and multiple dosing regimens as well as any adverse effect due to multiple dosing at 3 mg/eye (Ia) or 1.5 (Ib) were investigated. In phase II test articles were administered twice during the study period on days 1 and 15. Eyes were examined with an indirect ophthalmoscope and fundus camera.

### Experimental Procedures

Adult male cynomolgus monkeys (Macaca fascicularis) age 2-7 years old were used to test the efficacy of BOL-303050-X, BOL-303054-X and BOL-303055-X. Lucentis® (ranibizumab) was used as historic control.

### Phase Ia

The right eyes of four monkeys that were previously used for non-ocular studies were injected intravitreally (50 μL) with the aqueous formulation (PBS containing 0.15% Tween 80) of BOL-303050-X (3 mg/eye), BOL-303051-X (3 mg/eye), BOL-303054-X (3 mg/eye) or BOL-303055-X (3 mg/eye) into the inferior quadrant of the vitreous and the left eyes received (50 μL) the vehicle on days 1 and 15. Ocular examinations were carried out on these animals at baseline and days 3, 8 and 11 post-injection. On day 15, the left eyes of monkeys I01111 and I01112 were injected (50 μL) with oil formulation vehicle only containing Miglyol 812 into the superior quadrant of the vitreous and the left eyes were examined on days 15, 17 and 22. Phase Ia study was terminated on day 22.

### Phase Ib

The right eyes of four monkeys that were previously used for non-ocular studies were injected intravitreally (50 μL) with the oil formulation (Miglyol 812) of BOL-303050-X (1.5 mg/eye), BOL-303051-X (1.5 mg/eye), BOL-303054-X (1.5 mg/eye) or BOL-303055-X (1.5 mg/eye) into the superior quadrant of the vitreous and the left eyes received (50 μL) the vehicle on days 1, 15 and 29. Ocular examinations were carried out on these animals at baseline and days 3, 8, 11, 15, 17, 29, 31 and 37 post-injection. Phase 1b study was terminated on day 37.

### Phase II

### Laser-Induced Choroidal Neovascularization

Both eyes of 46 monkeys were examined at baseline and then 42 animals were randomized into 5 groups. On Phase II Day 8 of Dosing, the animals were anesthetized with ketamine and dexmedetomidine and the macula of each eye of the animals in Groups 1 through 6 underwent laser treatment by a retinal surgeon with a 532 nm diode green laser (OcuLight GL, IRIDEX Corp. Inc., Mountain View, Calif.) using a slit lamp delivery system and a Kaufman-Wallow (Ocular Instruments Inc, Bellevue, Wash.) plano fundus contact lens with Gonak (2.5%) as the coupling agent. Nine laser spot areas were symmetrically placed in the macula of each eye. The laser parameters included a 75 micron spot size and 0.1 second duration. The power setting used was assessed by the ability to produce a blister and a small hemorrhage. Unless hemorrhage was observed with the first laser treatment, a second laser spot was placed adjacent to the first following the same laser procedure (except the wattage was adjusted). For areas not adjacent to the fovea, the initial power setting was 500 mW; if a second spot was placed, the power was set to 650 mW. For the area adjacent to the fovea the power settings were 400 mW (initial treatment) and 550 mW (second treatment). At the discretion of the retinal surgeon, power settings were adjusted based on observations at the time of laser. Power setting used for each spot was documented as well as number of burns per spot and whether hemorrhage occurred.

### Dose Administration

Once the phase Ib study was completed, and it was established that the drug suspension in oil would not interfere with either laser administration, evaluation of CNV or cause adverse effects, phase II was initiated. On days 1, and 15 both eyes of monkeys received 50 μL of Lucentis (0.5 mg/eye), vehicle (0 mg/eye), BOL-303050-X (1.5 mg/eye), BOL-303054-X (1.5 mg/eye) or BOL-303055-X (1.5 mg/eye) (Table 2). Intravitreous injection was performed for each by eye aiming towards mid-vitreous body. Dosing in the right eye was alternated for successive doses (as applicable) at approximately the 11 o'clock and 10 o'clock positions. Dosing in the left eye was alternated for successive doses (as applicable) from approximately the 1 o'clock and 2 o'clock positions.

Animals were anesthetized with ketamine and dexmedetomidine. A topical anesthetic (0.5% proparacaine) was instilled in each eye before dose administration. A wire speculum was used to retract the eyelids. The eyes were cleaned with a 2.5% povidone iodine solution and then rinsed with sterile saline. Each animal was dosed using 1 cc tuberculin syringes and 30 gauge needles.

TABLE 2
Test Article Administration

| Group | n | Right Eye | Left Eye |
|---|---|---|---|
| 1 | 9 | BOL-3030YY-X | BOL-3030YY-X |
| | | Vehicle | Vehicle |
| 2 | 9 | BOL-303050-X | BOL-303050-X |
| 3 | 8 | BOL-303054-X | BOL-303054-X |
| 4 | 9 | BOL-303055-X | BOL-303055-X |
| 5 | 7 | Lucentis ® | Lucentis ® |

### Clinical Ophthalmic Examination (OE)

Animals were examined at baseline, on days 1, 3, 7, 15, and 31 of Dosing Phase II and on the day of scheduled sacrifice (day 38). On days of dose administration the ophthalmic examination was done prior to dosing.

### Intraocular Pressure (TOP)

IOP was done in conjunction with the OE using a Tono-pen on anesthetized monkeys on days 1, 3, 7, 15, and 31 of dosing Phase II and on the day of scheduled sacrifice (day 38).

### Fluorescein Angiography (FA)

FA was performed at baseline, on days 22, 29 and 37 (approximately 14, 21 and 28 days post-laser). Digital ocular photographs were taken in conjunction with FA. The FA procedure is well known by people skilled in the art of ophthalmology.

Evaluation of FA was done according to the following grading system for evidence of excessive permeability (fluorescein leakage) or any other abnormalities.

| Lesion Grade | Definition |
|---|---|
| I | No hyperfluorescence |
| II | Hyperfluorescence without leakage |
| III | Hyperfluorescence early or mid-transit |
| | and late leakage |
| IV | Bright hyperfluorescence early or mid- |
| | transit and late leakage beyond borders of |

Grade IV lesions are considered to be clinically significant as these most closely resemble the active forms of classical CNV seen in various human retinal disorders, including age-related macular degeneration. Comparison of the incidence of Grade IV lesions between treatment groups was evaluated.

### Ocular Photographs (OP)

Ocular photographs were collected in all animals in conjunction with laser induction of CNV (day 8) and FA.

### Termination

Experiments were terminated on day 38 and eyes that had undergone testing were collected and stored at room temperature in 70% ethanol.

### Data Analysis

### Statistical Methods

Grade IV lesions were analyzed using either Fisher's exact probability test (2-Tail) or Chi square test (χ2). For the analysis of delta changes in TOP a two-way ANOVA with repeated measures followed by the contrast procedure was performed on Box-Cox transformed data if needed. Unless indicated otherwise, data are presented as means±SEM unless stated otherwise. P<0.05 was considered statistically significant.

### Results

Unlike the aqueous formulation of compounds, the oil formulation prevented the dispersion of the integrin antagonist in the vitreous allowing the eyes to be lasered and evaluated until the Phase II studies.

The laser-induced monkey CNV model worked as expected. Lucentis, the historical control, inhibited all grade IV lesions (0:126) at all the studied time points (Table 3).

With respect to the study, the vehicle-treated group had a 32.10% (52:162), 25.93% (42:162) and 18.52% (30:162) incidence of grade IV lesions on days 22, 29 and 37, respectively (FIG. 3).

The incidence of grade IV lesions in eyes treated with BOL-303050-X was reduced compared to vehicle-treated eyes. There was a 4.05% (6:148), 2.67% (4:150) and 1.85% (3:162) incidence of grade IV lesions on days 22, 29 and 37, respectively (FIG. 3). These differences were statistically significant (p<0.0001) for the three days when compared with the incidences for the vehicle control group at the 22, 29 and 37 days. All grade IV lesions observed in the BOL-303050-X treated eyes were located in the same laser spot position for all 5 individual monkeys. The significance of this observation is under investigation.

Eyes treated with integrin antagonists BOL-303054-X or BOL-303055-X appeared to have higher incidences of grade IV lesions compared to the vehicle control. These differences for BOL-303054-X were not statistically significant at days 22 and 29, whereas there was a statistically significant (p=0.028) difference on day 37. In the case of BOL-303055-X (47.53%, 43.21% and 44.44%) statistically significant differences were observed (P<0.0001) when compared with the incidences for the vehicle control group at the 22, 29 and 37 days (FIG. 3).

An analysis of the overall effects in the study was also conducted by evaluating the incidence of grade IV lesions observed at all the time points. Table 4 shows that the overall grade IV lesions were significantly reduced in BOL-303050-X-treated eyes, while the incidences of grade IV lesions were significantly increased in the BOL-303055-X-treated eyes versus vehicle control group. Overall incidences of grade IV in BOL-303054-X-treated eyes were not statistically significant indicating that they were similar to the vehicle control group.

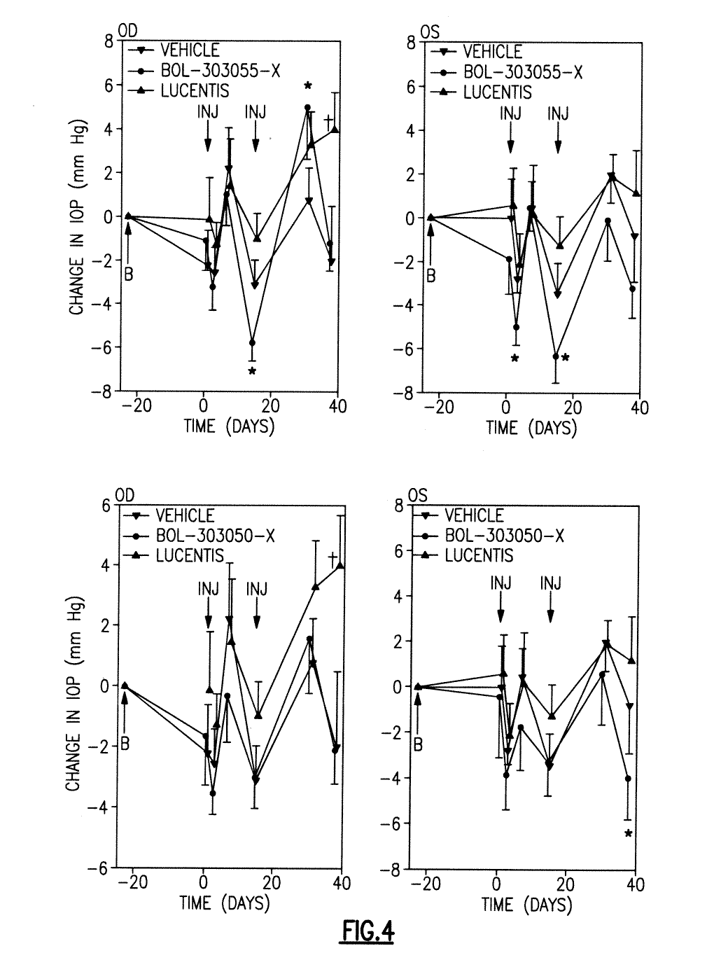

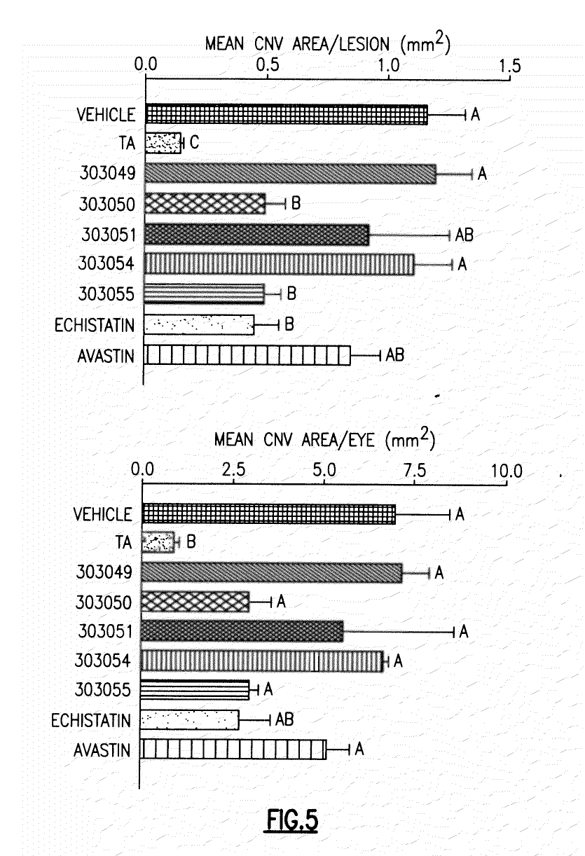

This study was not designed to evaluate the effect of test articles on IOP, but some statistically significant changes to IOP were observed. Monkeys treated with intravitreal injections of BOL-303055-X appeared to have reduced IOP in the right eyes on day 15 and in the left eyes on days 3 and 15 and these differences were statistically significant when compared with baseline (FIG. 4, top panel). In addition, IOP was significantly increased by 4 mm Hg in the right eyes of BOL-303055-X-treated monkeys on day 31, and this difference was also statistically significant compared to baseline, eventually returning to baseline by day 38. It is important to note, that there were no statistically significant differences when the BOL-compound-treated groups were compared with their vehicle control at each individual time point. This observation indicates that these changes may be related to the procedure rather than the pharmacological effects of the compounds. A late increase in IOP (approximately 4 mm Hg) was also observed in the right eyes of Lucentis®-treated animals versus BOL3030YY-X vehicle control on day 38 (FIG. 4).

Intravitreal administration of BOL-303050-X significantly reduced IOP by 4 mm Hg versus baseline on day 38 only (FIG. 4, bottom panel). Similarly to the data with BOL-303055-X, no other changes in IOP were observed when comparing with the vehicle control, suggesting more a procedure change in IOP that a pharmacological effect of the agent.

Mild anterior chamber inflammation (cells and flare) was only observed 3 days post IVT injections. Moderate anterior chamber inflammation was observed in the left eye of monkey #I02127 (BOL-303054-X-treated) that had developed endophthalmitis on day 29 which lasted until day 38 (data not shown).

Eyes injected with the drug suspensions appeared to have higher vitreal cell scores than eyes injected with the oil vehicle or Lucentis (Table 4). However, examiner(s) was unable to distinguish between cells or dispersed drug in the vitreous. No other ocular findings were reported (except the left eye of monkey #102127) in any of the eyes injected with the test articles.

### Summary and Conclusion

Prophylactic treatment of eyes with BOL-303050-X in oil formulation reduced the number of grade IV lesions in the laser-induced CNV in monkeys in a statistically significant manner. No drug-related ocular findings were reported.

BOL-303050-X inhibits CNV in the monkey and is a good candidate for treating exudative AMD in patients. Also combination therapy with Lucentis® or Avastin® should be explored more fully.

Further studies to determine the mechanism for TOP changes or increased vitreal cell scores in eyes injected with drug suspension are desirable.

TABLE 2
Incidence of CNV lesions according to grade in eyes treated with Lucentis,
vehicle, BOL-303050-X, BOL-303054-X and BOL-303055-X. Test agents were
injected intravitreally into both eyes on days 1 and 15 and lesions were scored on days
22, 29 and 37 post-injection.

| | Lesion Grade (% Total) | | | |
|---|---|---|---|---|
| Treatment | I | II | III | IV |

Day 22

| | | | | |
|---|---|---|---|---|
| Lucentis | 97:126 (76.98) | 20:126 (15.87) | 9:126 (7.14) | 0:126 (0) |
| Vehicle | 89:162 (54.94) | 10:162 (6.17) | 11:162 (6.79) | 52:162 (32.1) |
| BOL-303050-X | 126:148 (85.14) | 12:148 (8.11) | 4:148 (2.70) | 6:148 (4.05) |
| BOL-303054-X | 79:133 (59.40) | 1:133 (0.75) | 8:133 (6.01) | 45:133 (33.83) |
| BOL-303055-X | 73:162 (45.06) | 6:162 (3.7) | 6:162 (3.70) | 77:162 (47.53) |

Day 29

| | | | | |
|---|---|---|---|---|
| Lucentis | 96:126 (76.19) | 22:126 (17.46) | 8:126 (6.35) | 0:126 (0) |
| Vehicle | 92:162 (56.79) | 9:162 (5.56) | 19:162 (11.73) | 42:162 (25.93) |
| BOL-303050-X | 132:150 (88.00) | 11:150 (7.33) | 3:150 (2.00) | 4:150 (2.67) |
| BOL-303054-X | 80:133 (60.15) | 5:133 (3.76) | 2:133 (1.50) | 46:133 (34.59) |
| BOL-303055-X | 75:162 (46.3) | 5:162 (3.09) | 12:162 (7.41) | 70:162 (43.21) |

Day 37

| | | | | |
|---|---|---|---|---|
| Lucentis | 118:126 (93.65) | 8:126 (6.35) | 3:126 (1.85) | 0:126 (0) |
| Vehicle | 109:162 (67.28) | 17:162 (10.49) | 6:162 (3.70) | 30:162 (18.52) |
| BOL-303050-X | 136:162 (83.95) | 15:162 (9.26) | 8:162 (4.94) | 3:162 (1.85) |
| BOL-303054-X | 83:136 (61.03) | 6:136 (4.41) | 5:136 (3.68) | 41:136 (30.15) |
| BOL-303055-X | 80:162 (49.38) | 7:162 (4.32) | 5:162 (3.68) | 72:162 (44.44) |

TABLE 3
Overall grade IV lesions in eyes treated with vehicle, BOL-303050-X,
BOL-303054-X and BOL-303055-X. Incidences were calculated as
the sum of the number of grade IV lesions observed in all the
days relative to the sum of all the lesions examined.

| Treatment | Overall Grade IV lesions (%) | |
|---|---|---|
| Vehicle | 142:486 | (25.51) |
| BOL-303050-X | 13:460 | (2.83)* |
| BOL-303054-X | 132:402 | (32.84) |
| BOL-303055-X | 219:486 | (45.06)* |

*P < 0.05 versus vehicle control group using the Chi square test (χ2).

TABLE 4
Data showing vitreal cell scores in eyes treated with Lucentis, vehicle, BOL-303050-X, BOL-303054-X and
BOL-303055-X. Test agents were injected intravitreally into both eyes on days 1 and 15 and eyes were examined
(using a limited set of read outs of the modified McDonald-Shadduck system) on days 3, 7, 15, 31 and 38. Examiner(s)
was not able to distinguish between cells or dispersed test material in the vitreous of eyes injected with the drug
suspension. Therefore, the scores indicated below may not reflect actual cell numbers in the vitreous.
Days 3-15

| | | | Day 3 | | | | | | Day 7 | | | | | | | | | | | |
|---|---|---|---|---|---|---|---|---|---|---|---|---|---|---|---|---|---|---|---|---|
| Treatments | n | Grade | 0 | Trace | 1 | 2 | 3 | 4 | 0 | Trace | 1 | 2 | 3 | 4 | Day 15 | | | | | |
| Lucentis | 14 | | 9 | 5 | 0 | 0 | 0 | 0 | 5 | 5 | 1 | 1 | 2 | 0 | 5 | 5 | 1 | 3 | 0 | 0 |
| Vehicle | 18 | | 14 | 4 | 0 | 0 | 0 | 0 | 10 | 4 | 1 | 1 | 1 | 0 | 10 | 4 | 1 | 3 | 0 | 0 |
| BOL-303050-X | 18 | | 8 | 4 | 5 | 0 | 0 | 1 | 1 | 0 | 4 | 6 | 2 | 5 | 1 | 2 | 8 | 2 | 1 | 4 |
| BOL-303054-X | 16 | | 9 | 5 | 1 | 1 | 0 | 0 | 2 | 2 | 2 | 2 | 4 | 6 | 0 | 0 | 6 | 2 | 0 | 8 |
| BOL-303055-X | 18 | | 11 | 6 | 1 | 0 | 0 | 0 | 0 | 5 | 3 | 6 | 3 | 1 | 0 | 4 | 7 | 3 | 1 | 3 |

Days 31 & 38

| | | | Day 31 | | | | | | Day 38 | | | | | |
|---|---|---|---|---|---|---|---|---|---|---|---|---|---|---|
| Treatments | n | Grade | 0 | Trace | 1 | 2 | 3 | 4 | 0 | Trace | 1 | 2 | 3 | 4 |
| Lucentis | 14 | | 4 | 6 | 0 | 4 | 0 | 0 | 7 | 5 | 1 | 1 | 0 | 0 |
| | | | 5 | 7 | 2 | 4 | 0 | 0 | 6 | 7 | 5 | 0 | 0 | 0 |
| Vehicle | 18 | | 0 | 0 | 1 | 6 | 8 | 3 | 0 | 0 | 2 | 4 | 4 | 8 |
| BOL-303050-X | 18 | | 0 | 0 | 2 | 1 | 1 | 11 | 0 | 0 | 1 | 0 | 3 | 12 |
| BOL-303054-X | 16 | | 0 | 1 | 1 | 8 | 2 | 6 | 0 | 0 | 0 | 3 | 2 | 13 |
| BOL-303055-X | 18 | | 5 | 7 | 2 | 4 | 0 | 0 | 6 | 7 | 5 | 0 | 0 | 0 |

### Testing 3: Evaluation of Intravitreal Administration of BOL-303049-X, BOL-303050-X, BOL-30305′-X, BOL-303054-X AND BOL-303055-X on Laser-Induced Choroidal Neovascularization in Rats

### Purpose

To evaluate the effect of intravitreal administration of five integrin antagonists, echistatin and Avastin® in the rat laser-induced CNV.

### Materials and Methods:

### Study Design

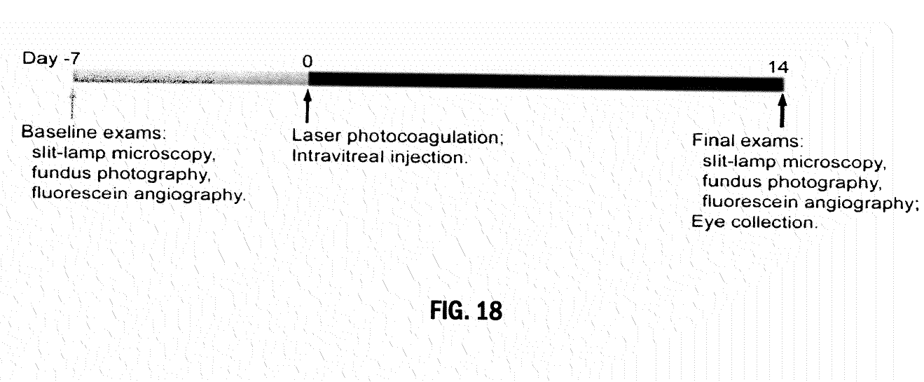

See FIG. 18.

### Animals

Fifty six male Brown Norway rats, approximately 200-300 grams of body weight and two months of age, were used in this study. Animals were randomly allocated into ten groups as shown in Table 6. This study was performed in two cohorts and only one eye of each rat was used.

TABLE 6
Test Article Administration

| | Number | | Doses for |
|---|---|---|---|
| Group | of rats | Product information | intravitreal injection |
| 1 | 5 | Vehicle of BOL-3030XX-X, Bausch & Lomb, | 5 μL/eye |
| | | Lot 2892EP147A | |
| 2 | 6 | BOL-303049-X, Bausch & Lomb, Lot | 200 μg/5 μL/eye |
| | | 2892EP148-1, Molecular weight 603.72, | (40 mg/mL) |
| | | Formula C₃₂H₄₁N₇O₅ | |
| 3 | 6 | BOL-303050-X, Bausch & Lomb, Lot | 200 μg/5 μL/eye |
| | | 2892EP148-2, Molecular weight 595.72, | (40 mg/mL) |
| | | Formula C₂₉H₃₇N₇O₅S | |
| 4 | 6 | BOL-303051-X, Bausch & Lomb, Lot | 200 μg/5 μL/eye |
| | | 2892EP148-3, Molecular weight 565.72, | (40 mg/mL) |
| | | Formula C₃₀H₄₃N₇O₄ | |
| 5 | 6 | BOL-303054-X, Bausch & Lomb, Lot | 200 μg/5 μL/eye |
| | | 2892EP148-4, Molecular weight 575.67, | (40 mg/mL) |
| | | Formula C₃₀H₃₇N₇O₅ | |
| 6 | 6 | BOL-303055-X, Bausch & Lomb, Lot | 200 μg/5 μL/eye |
| | | 2892EP148-5, Molecular weight 573.7, | (40 mg/mL) |
| | | Formula C₃₁H₃₉N₇O₄ | |
| 7 | 6 | Kenalog ®-40 (triamcinolone acetonide), | 200 μg/5 μL/eye |
| | | Bristol-Myers Squibb, NDC 0003-0293-20, | (40 mg/mL) |
| | | Lot 8G43208 | |
| 8 | 6 | Avastin ® (bevacizumab), Genentech, NDC | 125 μg/5 μL/eye |
| | | 50242-060-01, Lot 746967 | (25 mg/mL) |
| 9 | 6 | Echistatin, Sigma-Aldrich, Product number | 5 μg/5 μL/eye |
| | | E1518, Lot 127K4050 | (1 mg/mL) |
| 10 | 3 | Vehicle of Avastin, Bausch & Lomb, Lot | 5 μL/eye |
| | | 2892EP148A | |

### Baseline Ocular Examination

Both eyes of each rat were dilated with 1-2 drops of each 2.5% phenylephrine hydrochloride and 1% tropicamide. After full pupil dilation, the animals were anesthetized with intramuscular injections of 50 mg/kg ketamine hydrochloride and 4 mg/kg xylazine mixture before ocular examination.

The anterior segment of the eye was examined using a slit-lamp biomicroscopy for excluding any animals with abnormalities that might interfere with the study. Fundus photography and fluorescein angiography (FA) were performed to document the health of the retina and the ocular circulation using a TRC 50EX fundus camera (Topcon). For FA, 10% sodium fluorescein was injected intravenously (50 mg/kg of body weight) through the tail vein or the vein under the incisor before the procedure.

### Laser Photocoagulation

The pupils were dilated and then the rats were anesthetized as described in baseline examination. The rats were positioned in front of a slit lamp system (Haag-Streit USA, Mason, Ohio). The fundus was visualized using an Ocular Fundus 5.4 mm Laser Lens (0 diopter, Ocular Instruments, Bellevue, Wash.) with 2.5% hydroxypropyl methylcellulose solution (Goniosoft®, Ocusoft Inc., Richamond, Tex.) as an optical coupling agent. A diode laser (OcuLight TX, IRIDEX, Mountain View, Calif.) was used for photocoagulation (532 nm wavelength, 120 mW power, 50 μm spot size and 0.05 seconds duration). For each right eye, a series of six photocoagulation sites were concentrically placed between the major retinal vessels at equal distance (about two optic discs) around the optic nerve head. Production of a subretinal vapor bubble at the time of laser treatment was used to confirm the rupture of the Bruch's membrane. If a significant retinal or subretinal hemorrhage occurred during the laser photocoagulation, then the left eye was be photocoagulated and used as the experimental eye.

### Intravitreal Injections

Immediately after placement of laser photocoagulation, the experimental eyes received an intravitreal injection of either a compound or a vehicle as described in Table 5. Following the disinfection of ocular surface with 5% betadine and 0.9% saline solutions, the injections were performed with aid of a surgical microscope using 0.3 mL syringes with a 31-gauge ½-inch long needle (Ultra-Fine II™ Insulin syringe, BD, Franklin Lakes, N.J.).

### Final Ocular Examination

Two weeks after photocoagulation, all rats were examined under anesthesia and pupil dilation with slit-lamp biomicroscopy, fundus photography and FA as described in the baseline examination. The fluorescein leakage was semi-quantitatively graded for the unobstructed laser spots as 1-4. Grade 1 lesions had no hyperfluorescence. Grade 2 lesions exhibited hyperfluorescence without leakage. Grade 3 lesions exhibited hyperfluorescence in the early or midtransit images and late leakage. Grade 4 lesions showed bright hyperfluorescence in the transit images and late leakage beyond treated areas. The relative distribution and mean scores of FA grades were determined within each experimental group.

### Tissue Collection and Preparation

The eyes were collected on day 14 and immediately fixed in 4% paraformaldehyde in PBS (pH 7.4) for one and half hours, and then transferred into cold PBS and kept at 4° C. for further processing.

### Immunohistochemistry and Confocal Microscopy

The flat mounts were placed in a 48-well cell culture cluster, rinsed with immunohistochemistry (IHC) buffer (0.5% BSA, 0.2% Tween 20, 0.05% sodium azide in PBS) at 4° C. for 2 hours, then incubated in labeling buffer, containing 1:100 dilutions of each 1 μg/μL isolectin IB4 conjugated with Alexa Fluor 568 and 0.2 units/μL phalloidin conjugated with Alexa Fluor 488 (Invitrogen-Molecular Probes, Eugene, Oreg.) in IHC buffer. The incubation was performed in a humidified dark chamber with gentle rotation at 4° C. overnight. On the second day, the retinal pigment epithelium (RPE)-choroidsclera complexes were washed with cold IHC buffer, mounted with ProLong® Gold antifade reagent, covered with circular cover glass, and sealed with clear nail polish. Fluorescent images were collected with a confocal microscope (FluoView FV 1000 confocal microscope, Olympus, Center Valley, Pa.) and a 4×, 0.16 numerical aperture objective lens. Fluorescent signals for Alexa Fluor 488 and Alexa Fluor 568 were collected by using a sequential scan mode to reduce bleed-through. All images were collected at a 1024×1024-pixel resolution and a depth of 24 bits per channel. The laser sites were identified by merging images of the green (phalloidin labeling RPE) and the red (isolectin IB4 labeling CNV) channels. CNV complexes were identified using the red channel (isolectin), and their areas were quantified using the FluoView software (Version 1.7a).

### Data Analysis

Average FA scores and areas of CNV complexes in each group were presented as mean±SEM, unless otherwise indicated. They were analyzed using a one-way ANOVA followed by a Tukey-Kramer test after Box-Cox transformations. All statistics were performed utilizing the statistical analysis software JMP (SAS Institute, Cary, N.C.). p values ≦0.05 were considered statistically significant.

### Results:

BOL-303050-X-, BOL-303055-X-, triamcinolone acetonide- and echistatin treated eyes developed less area of CNV

Merged images of phalloidin-labeled RPE cells (green) and isolectin-labeled endothelial cells (red) clearly showed the laser lesion sites around the optic nerve head. Unfortunately, because of a new lot of isolectin IB4 that had a problem in quality, the 28 eyes in Cohort B could not be labeled for visualization of CNV. Therefore, only the 28 eyes in Cohort A had been included in the analysis of CNV area. As the data from two groups of vehicles did not have statistically significant difference, those two groups were combined together and used as the vehicle-treated control group for the analysis.

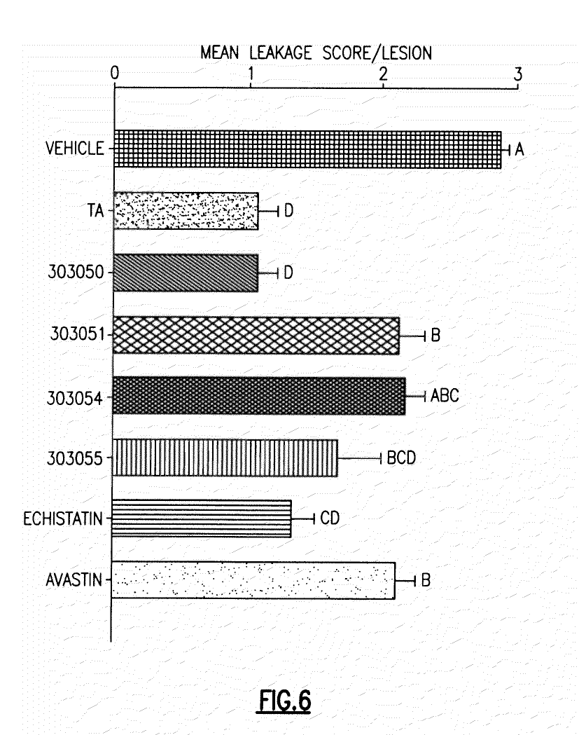

Fourteen days post-photocoagulation, the phalloidin-labeled actin-cytoskeleton showed thinner fibers around the lesion sites in the TA-, BOL-303050-X-, BOL-303055-X-, and echistatin-treated eyes than the vehicle-treated eyes (FIG. 5), implying reduced proliferation and migration of RPE cells in those compound-treated eyes than the vehicle-treated eyes. Robust isolectin-labeled CNV complexes were visible in the vehicle-treated controls, with well-defined radial array of new vessels. In comparison, tightly circumscribed circular regions were uniformly demonstrated in the TA-treated eyes, correlating with the disruption of the normal morphology of Bruch's membrane and RPE cells. Although a densely packed mass with isolectin-positive labeling filled the circular defect, no isolectin-labeled vascular structures were observed in the TA-treated eye. Fewer new vessels were also observed in BOL-303050-X-, BOL-303055-X- and echistatin-treated eyes. In BOL-303050-X-treated eyes, the development of CNV was completely inhibited in a small portion of lesions.

Quantitative measurement of CNV area was made on the isolectin-labeled flat mounts using the FluoView software. Compared to the vehicle-treated controls, TA-, BOL-303050-X-, BOL-303055-X-, and echistatin-treated eyes developed smaller area of CNV (FIG. 5). These differences were statistically significant. These data indicate that intravitreal injection of TA, BOL-303050-X, BOL-303055-X and echistatin significantly inhibit the development of experimental CNV induced by laser photocoagulation.

BOL-303050-X-, TA- and echistatin-treated eyes showed less leakage of fluorescein

In many eyes, the view of fundus was obscured by testing articles suspended in the vitreous. Posterior synechiae of iris occurred in several eyes injected with BOL-303049-X or BOL-303055-X, which made it more difficult to observe the fundus. In BOL-303049-X-treated eyes, only one out of the six eyes was barely observable for the major retinal vessels, and none of them were gradable for FA leakage. The number of FA gradable eyes and the distribution of FA grades were summarized in Table 7.

TABLE 7
Relative Distribution of FA Grades for Unobstructed Lesions

| | |
|---|---|
| | Number of |
| Qualified | lesions |
| samples | in each grade |

| Group | Eye | Lesion | 4 | 3 | 2 | 1 |
|---|---|---|---|---|---|---|
| Vehicle | 7 | 42 | 38 | 3 | 1 | 0 |
| TA | 3 | 18 | 0 | 4 | 11 | 3 |
| BOL-303049-X | 0 | 0 | 0 | 0 | 0 | 0 |
| BOL-303050-X | 4 | 24 | 2 | 4 | 12 | 6 |
| BOL-303051-X | 4 | 24 | 10 | 9 | 3 | 2 |
| BOL-303054-X | 1 | 6 | 1 | 5 | 0 | 0 |
| BOL-303055-X | 1 | 6 | 1 | 2 | 3 | 0 |
| Echistatin | 3 | 18 | 2 | 3 | 12 | 0 |
| Avastin | 6 | 36 | 15 | 12 | 7 | 2 |

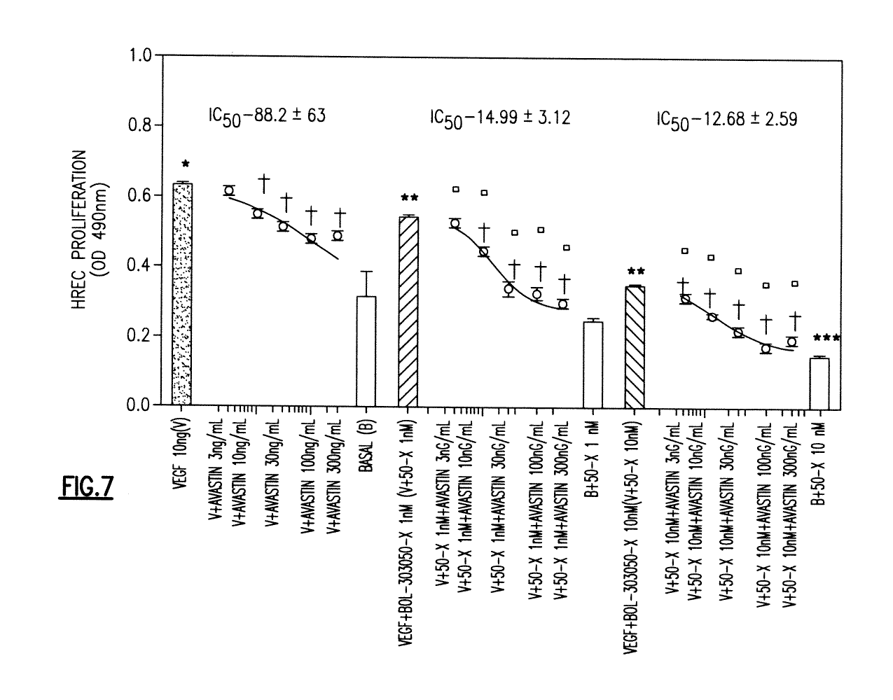

Fourteen days post-photocoagulation, the overall fluorescein leakage on lesions was noticeably less, fainter and more localized in the TA-, BOL-303050-X- and echistatin treated eyes, as compared to lesions in the vehicle-treated group. FA score analysis revealed smaller means in eyes treated with TA, BOL-303050-X, BOL-303055-X, echistatin, BOL-303051-X and Avastin when compared to the vehicle-treated controls (FIG. 6). However, only BOL-303050-X-, echistatin- and BOL-303055-X-treated eyes had no difference with TA-treated eyes statistically.

### Other Clinical Findings

Fourteen days after intravitreal injection, intraocular inflammation was observed in the anterior chamber in all eyes treated with BOL-303049-X and some of the eyes treated with BOL-303055-X, BOL-303054-X and BOL-303051-X (Table 8). Clinical signs included aqueous flare, exudates in the anterior chamber and iris posterior synechiae. The BOL-303049-X-treated eyes suffered the most severe inflammation. No inflammation was found in BOL-303050-X-treated eyes at Day 14. Retinal vascular congestion was also found in eyes treated with BOL-303049-X, BOL-303054-X and BOL-303055-X (Table 8). No retinal vascular congestion was noticed in eyes treated with BOL-303050-X or BOL-303051-X.

TABLE 8
Eyes With Anterior Chamber Inflammation or Retinal Vascular
Congestion at Day 14

| | Eyes with anterior | Eyes with retinal vascular |
|---|---|---|
| | chamber inflammation/ | congestion/vessel- |
| Tested articles | Total eyes | observable eyes |
| BOL-303049-X | 6/6 | 1/1 |
| BOL-303050-X | 0/6 | 0/5 |
| BOL-303051-X | 3/6 | 0/4 |
| BOL-303054-X | 4/6 | 4/5 |
| BOL-303055-X | 5/6 | 2/2 |
| Other test articles | 0/26 | 0/21 |

In eyes treated with vehicle, TA, echistatin and Avastin, no intraocular inflammation and retinal vascular congestion was recorded. Two eyes treated with echistatin developed cataract and another eye had a lot of small lesions leaking fluorescein in the retina. A similar leakage also occurred in one eye treated with vehicle.

### Summary and Conclusion

CNV can be consistently induced by laser photocoagulation in the Brown Norway rats. Intravitreal injection of TA effectively inhibits new vessel formation in this model.

Intravitreal injection of BOL-303050-X and BOL-303055-X significantly inhibits the development of CNV complexes in this animal model.

Neither inflammation nor retinal vascular congestion was observed in eyes injected with BOL-303050-X.

Intravitreal injection of 5 μg of echistatin significantly inhibits the development of CNV in this model. However, formation of cataract and retinal fluorescein leakage also occurred.

Intravitreal injection of 125 μg of Avastin® did not inhibit the development of CNV in this model.

### Testing 4: Study Evaluating the Sensitivity of VEGF-Treated Human Retinal Endothelial Cells to Avastin® in the Presence or Absence of BOL-303050-X

### Purpose

To evaluate the sensitivity of VEGF-treated human retinal endothelial cells to Avastin in the presence or absence of BOL-303050-X

### Materials and Methods

### Design

HREC were first cultured overnight in a 96-well plate coated with fibronectin. The next day, cells were serum-starved for 2 hours before treatment. The compound and Avastin® were prepared in 1% FBS-CS medium as shown in Table 9, and pre-incubated for 1 h before addition into appropriate wells. The cells were then cultured at 37° C., 5% CO₂, and 95% humidity for another 3 days. After that, cell proliferation was estimated by the MTS ((3-(4,5-dimethylthiazol-2-yl)-5-(3-carboxymethoxyphenyl)-2-(4-sulfophenyl)-2H-tetrazolium) assay, a cell metabolic activity assay. For a discussion of the MTS assay, see; e.g., http://en.wikipedia.org/wiki/MTT_assay or S. A. O'Toole et al., Cancer Detection and Prevention, Vol. 27, 47-54 (2003). Applicants' previous studies (not reported here) demonstrated that there is good correlation between the MTS assay and HREC cell numbers and between the MTS assay and the CyQuant assay in the estimation of cell number.

TABLE 9
Experimental Design and Schedules

| | | Day 2 | |
|---|---|---|---|
| | | All groups were pre-incubated for 1 h before being | |
| Group* | Day 1 | added into cells | Day 5 |
| 1 | HREC | Basal control (0.1% DMSO) | Evaluated cell |
| 2 | were | Basal + BOL-303050-X 1 nM | proliferation |
| 3 | cultured in | Basal + BOL-303050-X 10 nM | by MTS assay |
| 4 | a 96-well | VEGF 10 ng/ml | |
| 5 | plate O/N | VEGF + Avastin 3 ng/ml | |
| 6 | | VEGF + Avastin 10 ng/ml | |
| 7 | | VEGF + Avastin 30 ng/ml | |
| 8 | | VEGF + Avastin 100 ng/ml | |
| 9 | | VEGF + Avastin 300 ng/ml | |
| 10 | | VEGF + BOL-303050-X 1 nM | |
| 11 | | VEGF + Avastin 3 ng/ml + BOL-303050-X 1 nM | |
| 12 | | VEGF + Avastin 10 ng/ml + BOL-303050-X 1 nM | |
| 13 | | VEGF + Avastin 30 ng/ml + BOL-303050-X 1 nM | |
| 14 | | VEGF + Avastin 100 ng/ml + BOL-303050-X 1 nM | |
| 15 | | VEGF + Avastin 300 ng/ml + BOL-303050-X 1 nM | |
| 16 | | VEGF + BOL-303050-X 10 nM | |
| 17 | | VEGF + Avastin 3 ng/ml + BOL-303050-X 10 nM | |
| 18 | | VEGF + Avastin 10 ng/ml + BOL-303050-X 10 nM | |
| 19 | | VEGF + Avastin 30 ng/ml + BOL-303050-X 10 nM | |
| 20 | | VEGF + Avastin 100 ng/ml + BOL-303050-X 10 nM | |
| 21 | | VEGF + Avastin 300 ng/ml + BOL-303050-X 10 nM | |

*Four wells per group

### Materials

Integrin antagonist compounds: BOL-303050-X, free acid molecular weight of 595.73.

Compound stock solutions were made up in DMSO and stored at −20° C. To limit DMSO concentration to 0.1%, a 1000 times working concentration was prepared in DMSO for every final incubation concentration.

HREC were purchased from Cell System (Kirkland, Wash.). The cells are routinely cultured and maintained in complete medium containing 10% FBS before experiment. Cells were used at passage 5.

VEGF₁₆₅ (R & D system): The stock solutions were prepared in PBS containing 0.1% BSA and stored at −20° C. VEGF working solutions were prepared fresh in culture medium containing 1% FBS.

Avastin® (25 mg/ml, Genentech Inc.): The stock solution was stored at 4° C. Working solutions were prepared fresh in culture medium containing 1% FBS.

Immunoglobulin (50 μg/ml, Sigma): The stock solution was prepared in PBS and stored at 4° C.

### Other Reagents

| Reagent | Catalog # | Lot # | Vendor |
|---|---|---|---|
| CS-C medium | 4Z0-500 | 47805 | Cell System (Kirkland, WA) |
| Human fibronectin | F1904 | 0702053035 | Chemicon (Temecuia, CA) |
| Dimethyl sulfoxide (DMSO) | 41639 | 399326/1 | Fluka (St. Louis, MO) |
| Fetal bovine serum (FBS) | SH30070 | N/A | Hyclone (Logan, UT) |
| Normal human IgG | I4506 | 038K7540 | Sigma (St. Louis, MO) |

### Experiment

A 96-well plate was coated with human fibronectin (10 μg/ml) at 37° C. for 5 h. HREC were collected from flasks by EDTA-trypsin treatment and re-suspended in 10% FBS CS-C medium at 18,000 cells/ml. Cells (200 μl/well) were added to the plate and cultured overnight at 37° C., 5% CO₂, and 95% humidity. The next day, medium was aspirated and 200 μl/well serum-free medium was added for 2 h at 37° C., 5% CO₂, and 95% humidity.

VEGF, Avastin® and BOL-303050-X at different concentrations were prepared in 1% FBS CS medium as shown in Table 9. Normal IgG was added in such a way that all groups contained the same amount of IgG proteins. All medium solutions were incubated for 1 h at 37° C., 5% CO₂, and 95% humidity. After carefully aspirating the medium from cells, 200 μl of the medium containing different agents were added into appropriate wells. The cells were further cultured for 3 days.

At the end of incubation, the medium was carefully aspirated from cells and 120 μl of MTS working solution was added into all wells including four blank wells (without cells). The plate was incubated at 37° C., 5% CO₂, and 95% humidity for 1 hour and then read by a plate reader at 490 nM wavelength.

### Data Analysis

Median optical density (OD) was generated from different groups. Data were expressed as mean±SEM of the OD values. Statistical analysis was performed using two-way ANOVA followed by the contrast procedure after Box-Cox transformations if needed, and p<0.05 was considered statistically significant. To evaluate whether the effects of the combinations were either synergistic or additive, net inhibitions were calculated by subtracting the basal levels in the presence of VEGF from the individual treatments (Avastin® or integrin blockers) and the combinations multiplied by −1 to reverse the negative sign. A custom test in the fit model platform (equivalent to the Scheffé's test) was used to determine whether the net inhibition of the combination was significantly different from the sum of the net inhibitions obtained in the individual treatment. A statistically significant difference showing that the combination was greater than the sum of net individual treatment responses would indicate synergism, no significant differences would indicate additive effects, whereas significant differences below the sum of individual responses would indicate antagonistic effects. All the data from the individual wells were fitted to a re-parameterized four-parameter logistic equation to obtain IC₅₀ using similar methodology as that previously described and utilizing the statistical analysis software JMP (SAS Institute, Cary, N.C.).

### Results

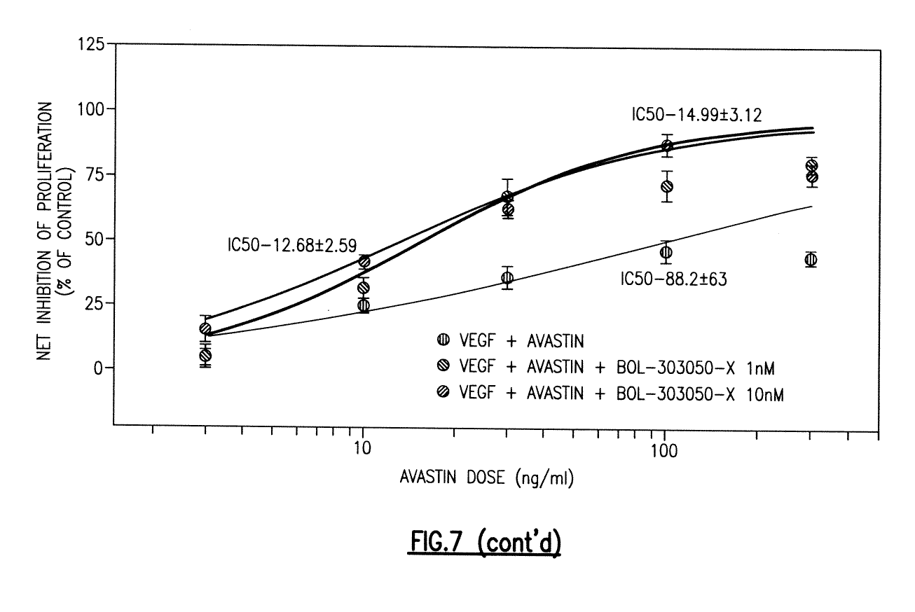

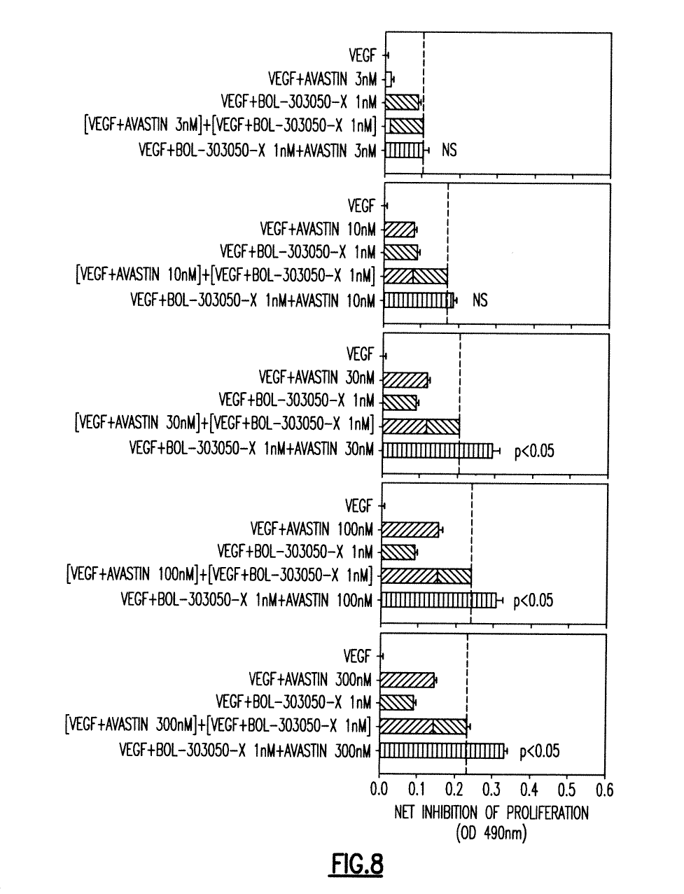

Under the stimulation of VEGF, the proliferation of HREC after 72 hours was increased by approximately 2-fold compared to the basal control and this difference was statistically significant (see FIG. 7A).

BOL-303050-X alone at 1 and 10 nM significantly reduced HREC proliferation in the presence of VEGF (see FIG. 7A). Under basal conditions, significant effects were only observed at 10 nM.

Avastin alone from 3-300 ng/ml showed a dose-dependent inhibition on VEGF-induced HREC proliferation, with statistical significance starting from the 10 ng/ml dose (see FIG. 7A).

In the presence of BOL-303050-X 1 nM, Avastin from 3-300 ng/ml showed a dose-dependent reduction of VEGF-induced HREC proliferation, with statistical significance starting from the 10 ng/ml dose (see FIG. 7A). Furthermore, the inhibiting effect of Avastin® at all doses was significantly greater in the presence of BOL-303050-X than in its absence (FIG. 7A).

In the presence of BOL-303050-X 10 nM, Avastin from 3-300 ng/ml showed a dose-dependent reduction of VEGF-induced HREC proliferation, with statistical significance starting from the 3 ng/ml dose. Furthermore, the inhibiting effect of Avastin® at all doses was significantly greater in the presence of BOL-303050-X than in its absence.

Further calculations showed that the presence of BOL-303050-X at 1 nM and 10 nM shifted the IC₅₀ of Avastin® from 88 to 15 and 13 ng/ml, respectively. These data indicate that BOL-303055-X at 1 nM and 10 nM enhance Avastin® potency by approximately 6 and 7-fold, respectively, suggesting a 6- to 7-fold improvement on the sensitivity of the cells to Avastin®. Overall, these data clearly point out a beneficial effect of BOL-303050-X by enhancing Avastin® potency.

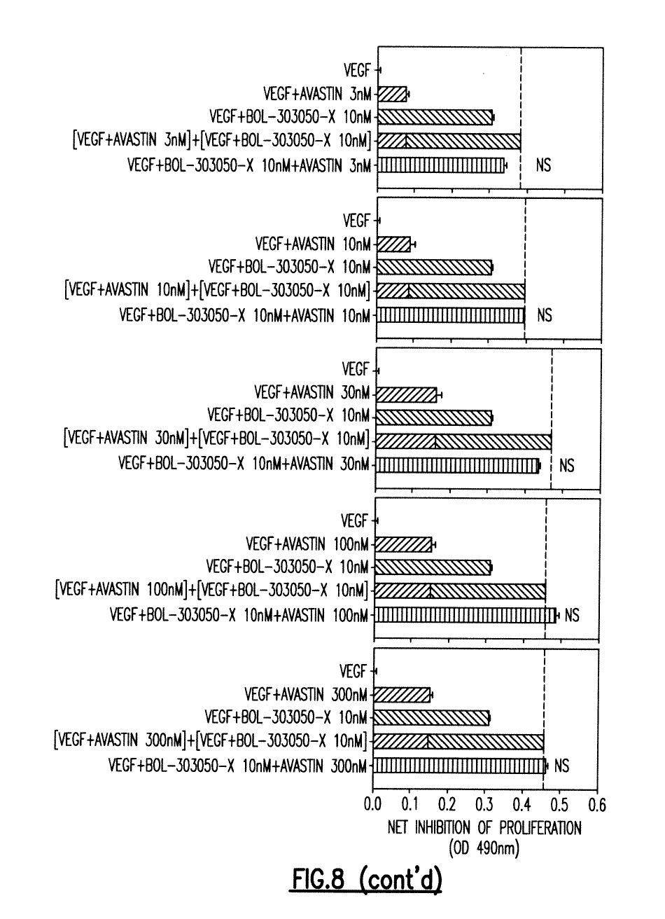

In order to assess whether the effects of BOL-303050-X and Avastin® were additive or synergistic, we calculated the net inhibition induced by either the integrin blockers or Avastin® alone and when given in combination. These data are presented in FIG. 8. From a theoretical point of view, additivity can be evaluated by analyzing whether the net effect of the combination is significantly greater than the sum of the individual responses. In FIG. 8, the third bar is a composite of the sum of individual responses and, therefore, represents the cutoff for additivity or synergy. Statistical evaluation using an analysis equivalent to the Scheffé's test indicates that the inhibition of proliferation obtained by Avastin® from 30 to 300 ng/ml, when given in combination with BOL-303050-X at 1 nM, is significantly greater (P<0.05) than that of the sum of the individual responses to Avastin® and to BOL-303050-X 1 nM alone. Therefore, these data indicate that BOL-303050-X at 1 nM exhibits synergistic effects with Avastin® to inhibit VEGF-induced proliferation of HREC (FIG. 8A). Based on the same criteria, BOL-303050-X at 10 nM exhibits additive effect with Avastin® at all doses to inhibit VEGF-induced HREC proliferation (FIG. 8B).

### Conclusion

Results from this study demonstrate a synergistic effect in the combination of BOL-303050-X and Avastin® on the inhibition of VEGF-induced proliferation of primary HREC. In addition, these data indicate that the addition of BOL-303050-X improves the potency of Avastin®, and, therefore, the sensitivity of the cells to the antibody, by approximately 6- to 7-fold under current conditions. Data from this and previous studies also indicate that the synergistic effect is highly dependent on the concentrations of the two parties—the integrin blockers and Avastin® in the assay system. This may in part be due to the window limitation of the in vitro model. Nevertheless, the beneficial effect of combining BOL-303050-X with Avastin® is clearly demonstrated.

One implication of the synergistic effect observed in this study is that we could use lower doses or fewer injections of this integrin blocker or Avastin® if we combine these two agents together in treating neovascular diseases. This may result in reducing potential side effects of the drugs in addition to saving costs and potentially increasing efficacy.

### Testing 5: Study Evaluating the Effects of Echistatin and Avastin Alone and in Combination on VEGF-Induced Proliferation of Human Retinal Endothelial Cells

### Purpose

To further investigate the effects of echistatin and Avastin alone and in combination on VEGF-induced proliferation of HREC.

### Materials and Methods

### Design

HREC were first cultured overnight in a 96-well plate coated with fibronectin. The next day, cells were serum-starved for 2 h before treatment. Medium containing different agents as shown in table below was pre-incubated for 1 h before being added to cells. The cells were cultured at 37° C., 5% CO₂, and 95% humidity for another 3 days. After that, cell proliferation was estimated by the MTS assay. In this study, we tested VEGF at 10 ng/mL (about EC₈₀ in study PH08169), echistatin at 2 nM (about EC₃₀ in study PH09032) and Avastin® 0.625, 1.25, 2.5, 5 and 10 ng/ml. Normal human IgG was also added to different groups in such a way that all groups contained the same amounts of IgG protein.

TABLE 10
Experimental Design and Schedules

| | | Day 2 | |
|---|---|---|---|
| | | All groups were pre-incubated for | |
| Group* | Day 1 | 1 h before adding into cells | Day 5 |
| 1 | HREC | Basal (1% FBS-medium) | Evaluate cell |
| 2 | were | Basal + Echistatin 2 nM | proliferation |
| 3 | cultured in | Basal + Avastin 0.625 ng/ml | by MTS |
| 4 | a 96-well | Basal + Avastin 1.25 ng/ml | assay |
| 5 | plate O/N | Basal + Avastin 2.5 ng/ml | |
| 6 | | Basal + Avastin 5 ng/ml | |
| 7 | | Basal + Avastin 10 ng/ml | |
| 8 | | VEGF 10 ng/mL | |
| 9 | | VEGF + Avastin 0.625 ng/ml | |
| 10 | | VEGF + Avastin 1.25 ng/ml | |
| 11 | | VEGF + Avastin 2.5 ng/ml | |
| 12 | | VEGF + Avastin 5 ng/ml | |
| 13 | | VEGF + Avastin 10 ng/ml | |
| 14 | | VEGF 10 ng/mL + Echistatin 2 nM | |
| 15 | | VEGF + Echistatin + Avastin 0.625 | |
| | | ng/ml | |
| 16 | | VEGF + Echistatin + Avastin 1.25 | |
| | | ng/ml | |
| 17 | | VEGF + Echistatin + Avastin 2.5 | |
| | | ng/ml | |
| 18 | | VEGF + Echistatin + Avastin 5 | |
| | | ng/ml | |
| 19 | | VEGF + Echistatin + Avastin 10 | |
| | | ng/ml | |

*4 wells per group

### Materials

Human retinal endothelial cells (HREC) were purchased from Cell System (Kirkland, Wash.). The cells are routinely cultured and maintained in complete medium containing 10% FBS before experiment. Cells at passage 4 were used.

VEGF₁₆₅ (R&D System) stock solution was prepared in PBS containing 0.1% BSA and stored at −20° C. VEGF working solutions were prepared fresh in culture medium containing 1% FBS.

Avastin®: The stock solution (25 mg/ml, Genentech Inc.) was stored at 4° C. Working solutions were prepared fresh in culture medium containing 1% FBS.

Echistatin: Echistatin (Sigma) stock solution was prepared in 0.1% BSA-PBS and stored at −20° C. Echistatin working solutions were prepared fresh in culture medium containing 1% FBS.

### Other Reagents:

| Reagent | Catalog # | Lot # | Vendor |
|---|---|---|---|
| CS-C medium | 4Z0-500 | 47805 | Cell System (Kirkland, WA) |
| Human fibronectin | F1904 | 0702053035 | Chemicon (Temecuia, CA) |
| Dimethyl sulfoxide (DMSO) | 41639 | 399326/1 | Fluka (St. Louis, MO) |
| Fetal bovine serum (FBS) | SH30070 | N/A | Hyclone (Logan, UT) |

### Experiment

A 96-well plate was coated with human fibronectin (10 μg/mL) for 5 hours. HREC were collected from flasks by EDTA-trypsin treatment and re-suspended in 10% FBS CS-C medium at 15,000 cells/mL. Cells (200 μL) were added to the plate and cultured overnight at 37° C., 5% CO₂, and 95% humidity. Next day, the medium was removed and 200 μl/well serum free medium was added.

Medium containing different agents was prepared in 1% FBS CS medium as shown in Table 10. All medium solutions were incubated for 1 h at 37° C. After carefully aspirating the medium from cells, 2004, of the medium containing the different agents was added into appropriate wells. The cells were cultured for another 3 days.

At the end of incubation, the medium was carefully aspirated from cells and 120 μl of MTS working solution was added into all wells including blank wells (without cells). The plate was incubated at 37° C., 5% CO₂, and 95% humidity for 1 hour and then absorbance was read by a plate reader at 490 nm wavelength.

### Data Analysis

Median optical density (OD) was generated from different groups. Data were expressed as mean±SEM of the OD values. Statistical analysis was performed using a one or two-way ANOVA using Box-Cox transformed data if needed, and P<0.05 was considered statistically significant. To evaluate whether the effects of the combinations were either synergistic or additive, net inhibitions were calculated by subtracting the basal levels in the presence of VEGF from the individual treatments (Avastin® and echistatin) and the combinations. A custom test in the fit model platform (equivalent to the Scheffé's test) was used to determine whether the net inhibition of the combination was significantly different from the sum of the net inhibitions obtained in the individual treatments. A statistically significant difference showing that the combination is greater than the sum of individual treatment responses would indicate synergism, no significant differences would indicate additive effects, whereas significant differences below the sum of individual responses would indicate antagonistic effects. All the data from the individual wells were fitted to a re-parameterized four-parameter logistic equation to obtain IC₅₀ using similar methodology as that previously described and utilizing the statistical analysis software JMP (SAS Institute, Cary, N.C.).

### Results

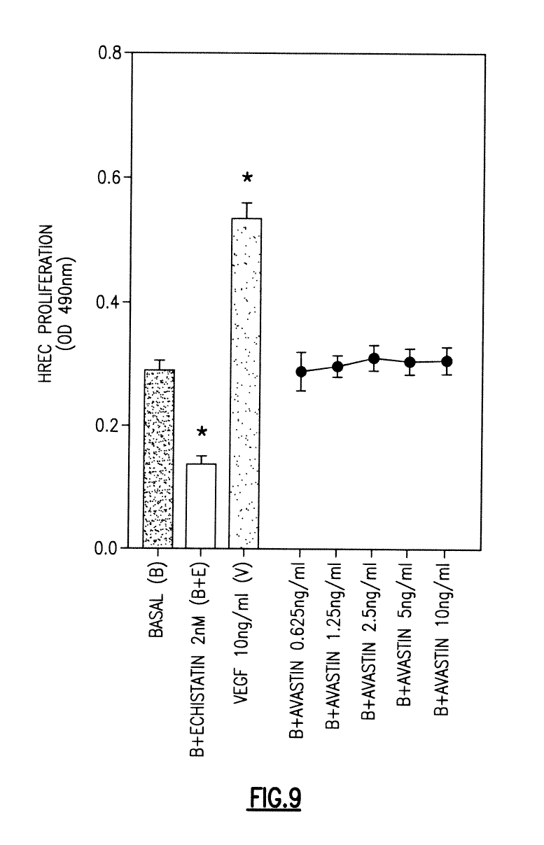

Under the stimulation of VEGF at 10 ng/mL, the proliferation of HREC was increased by approximately 2-fold compared to that of basal control and this difference was statistically significant (FIG. 9). Echistatin at 2 nM significantly inhibited HREC proliferation under basal conditions (FIG. 9). Avastin when given alone and at all doses tested showed no effect in altering HREC proliferation compared to basal control (FIG. 9).

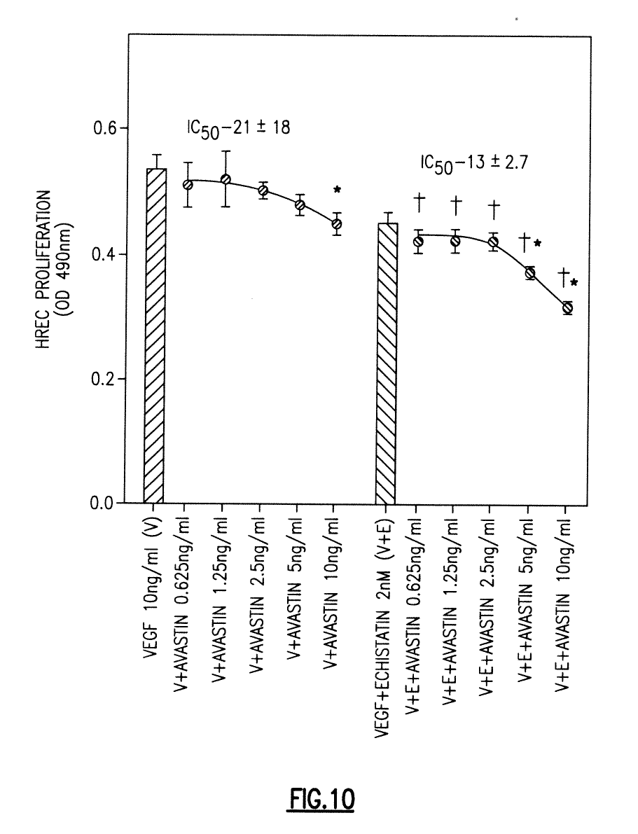

Avastin® from 0.625-10 ng/ml showed a dose-dependent inhibition on VEGF-induced HREC proliferation, with statistical significance at 10 ng/ml (FIG. 10). The inhibiting effect of echistatin at 2 nM on VEGF-induced proliferation was not statistically significant when compared to the VEGF control (FIG. 10). In the presence of echistatin, Avastin® from 0.625-10 ng/ml showed a dose-dependent inhibiting effect of VEGF-induced HREC proliferation, with statistical significance at both 3 and 10 ng/ml (FIG. 10). Furthermore, the inhibiting effect of Avastin® Avastin at all doses was significantly greater in the presence of echistatin than in its absence (FIG. 10). Further calculations showed that the presence of echistatin shifts the IC₅₀ of Avastin® from 21 to 13 ng/ml. These data indicate that a low dose of echistatin enhances Avastin potency by approximately 1.6-fold. Overall, these data clearly point out a beneficial effect of echistatin by enhancing Avastin® efficacy (FIG. 10 and Table 11).

TABLE 11
Summary of Estimated IC₅₀ of Avastin ® Alone or Avastin ®
Plus Echistatin on Inhibiting VEGF-Induced Proliferation of HREC

| Treatment | IC₅₀ ± SE (ng/ml) | 95% Conf. Limits |
|---|---|---|
| VEGF + Avastin | 21 ± 18 | 4.00-109 |
| VEGF + Avastin + Echistatin | 13 ± 2.7 | 9.0-20 |

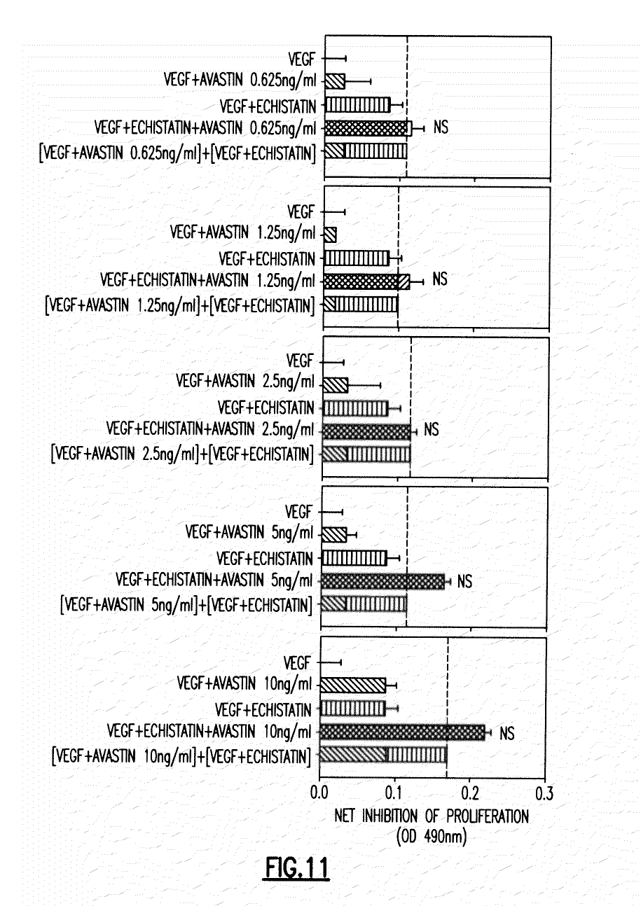

In order to assess whether the effects of echistatin and Avastin® were additive or synergistic, we calculated the net inhibition induced by either the integrin blockers or Avastin® alone and when given in combination. These data are presented in FIG. 11. From a theoretical point of view, additivity can be evaluated by analyzing whether the net effect of the combination is significantly greater than the sum of the individual responses. In FIG. 11, the last bar is a composite of the sum of individual responses and, therefore, represents the cutoff for additivity or synergy. Statistical evaluation using an analysis equivalent to the Scheffé's test indicates that the inhibition of proliferation obtained by Avastin® from 0.625 to 10 ng/ml, when given in combination with echistatin, is not significantly different (P>0.05) than that of the sum of the individual responses to Avastin® and to echistatin alone. Therefore, these data indicate that the echistatin exhibits additive effects with Avastin® to inhibit VEGF-induced proliferation of HREC.

### Conclusion

Results from this study demonstrate an additive effect in the combination of echistatin and Avastin® on the inhibition of VEGF-induced proliferation of primary human retinal endothelial cells. In addition, these data indicate that addition of echistatin improves the potency of Avastin® by approximately 1.6-fold. This study further confirms what we found in a previous study.

One implication of this additive effect is that we could use lower doses or fewer injections of this integrin blocker or Avastin® if we combine these two agents together in treating neovascular diseases. This may result in reducing potential side effects of the drugs in addition to saving costs and potentially increasing efficacy. At the minimum, the addition of an integrin blocker such as echistatin enhances the efficacy and potency of anti-angiogenic therapy with Avastin®.

### Testing 6: Study Evaluating the Effects of BOL-303055-X and Avastin Alone and in Combination on VEGF-Induced Proliferation of Human Retinal Endothelial Cells

### Purpose

To further investigate the effect of BOL-303055-X at one dose on the sensitivity of HREC to Avastin after VEGF-induced proliferation.

### Materials and Methods

### Design

HREC were first cultured overnight in a 96-well plate coated with fibronectin. The next day, cells were serum-starved for 2 hours. The medium containing different agents as shown in Table 12 was pre-incubated for 1 hour before being added into appropriate wells. The cells were further cultured at 37° C., 5% CO₂, and 95% humidity for another 3 days. After that, cell proliferation was estimated by the MTS assay—a cell metabolic activity assay (disclosed hereinabove). In this study, one dose of BOL-303055-X (1 nM; which was in previous studies showed synergistic effects) was combined with Avastin® at 5 different doses.

TABLE 12
Experimental Design and Schedules

| | | Day 2 | |
|---|---|---|---|
| | | All groups were pre-incubated for 1 h before being | |
| Group* | Day 1 | added into cells | Day 5 |
| 1 | HREC | Basal (0.1% DMSO) | Evaluated cell |
| 2 | were | Basal + Avastin 3 ng/ml | proliferation |
| 3 | cultured in | Basal + Avastin 10 ng/ml | by MTS assay |
| 4 | as 96-well | Basal + Avastin 30 ng/ml | |
| 5 | plate O/N | Basal + Avastin 100 ng/ml | |
| 6 | | Basal + Avastin 300 ng/ml | |
| 7 | | BOL-303055-X 1 nM | |
| 8 | | BOL-303055-X 1 nM + Avastin ng/ml | |
| 9 | | BOL-303055-X 1 nM + Avastin 10 ng/ml | |
| 10 | | BOL-303055-X 1 nM + Avastin 30 ng/ml | |
| 11 | | BOL-303055-X 1 nM + Avastin 100 ng/ml | |
| 12 | | BOL-303055-X 1 nM + Avastin 300 ng/ml | |
| 13 | | VEGF 10 ng/ml | |
| 14 | | VEGF + Avastin 3 ng/ml | |
| 15 | | VEGF + Avastin 10 ng/ml | |
| 16 | | VEGF + Avastin 30 ng/ml | |
| 17 | | VEGF + Avastin 100 ng/ml | |
| 18 | | VEGF + Avastin 300 ng/ml | |
| 19 | | VEGF + BOL-303055-X 1 nM | |
| 20 | | VEGF + Avastin 3 ng/ml + BOL-303055-X 1 nM | |
| 21 | | VEGF + Avastin 10 ng/ml + BOL-303055-X 1 nM | |
| 22 | | VEGF + Avastin 30 ng/ml + BOL-303055-X 1 nM | |
| 23 | | VEGF + Avastin 100 ng/ml + BOL-303055-X 1 nM | |
| 24 | | VEGF + Avastin 300 ng/ml + BOL-303055-X 1 nM | |

*Four wells per group

### Materials

Integrin antagonist compound: BOL-303055-X, free acid molecular weight: 573.70.

Compound stock solutions were made up in DMSO and stored at −20° C. To limit DMSO concentration to 0.1%, for every final incubation concentration, a 1000 times working concentration was prepared in DMSO.

Human retinal endothelial cells (HREC) were purchased from Cell System (Kirkland, Wash.). The cells are routinely cultured and maintained in complete medium containing 10% FBS before experiment. Cells at passage 4 were used in this study.

VEGF₁₆₅ (R&D System): The stock solutions were prepared in PBS containing 0.1% BSA and stored at −20° C. VEGF working solutions were prepared fresh in culture medium containing 1% FBS.

Avastin® (25 mg/ml, Genentech Inc): The stock solution was stored at 4° C. Working solutions were prepared fresh in culture medium containing 1% FBS.

### Other Reagents:

| Reagent | Catalog # | Lot # | Vendor |
|---|---|---|---|
| CS-C medium | 4Z0-500 | 47805 | Cell System |
| | | | (Kirkland. WA) |
| Human fibronectin | F1904 | 0702053035 | Chemicon |
| | | | (Temecuia, CA) |
| Dimethyl sulfoxide | 41639 | 399326/1 | Fluka (St. Louis, MO) |
| (DMSO) | | | |
| Fetal bovine serum | SH30070 | N/A | Hyclone (Logan, UT) |
| (FBS) | | | |

### Experiment:

A 96-well plate was coated with human fibronectin (10 μg/ml) for 5 h at 37° C., 5% CO₂, and 95% humidity. HREC were collected from flasks by EDTA-trypsin treatment and re-suspended in 10% FBS CS-C medium at 18,000 cells/ml. Cells (200 μl/well) were added to the plate and cultured for overnight at 37° C., 5% CO₂, and 95% humidity. Next day, medium was aspirated and serum-free medium was added to wells at 200 μl/well for 2 hours.

VEGF, Avastin® and BOL-303055-X at different concentrations were prepared in 1% FBS CS medium as shown in Table 12. Normal IgG was also added in such a way that all groups contained the same amount of IgG protein. All medium solutions were incubated for 1 h at 37° C., 5% CO2, and 95% humidity. After carefully aspirating the medium from cells, 200 μl of the medium containing different agents was added into appropriate wells. The cells were further cultured for 3 days.

At the end of incubation, the medium was carefully aspirated from cells and 120 μl of MTS working solution was added into all wells. The plate was incubated at 37° C., 5% CO₂, and 95% humidity for 1 hour and then read by a plate reader at 490 nM wavelength.

### Data Analysis

Median optical density (OD) was generated from different groups. Data were expressed as mean±SEM of the OD values. Statistical analysis was performed using a three-way ANOVA followed by the contrast procedure on log-transformed data, and P<0.05 was considered statistically significant. To evaluate whether the effects of the combinations were either synergistic or additive, net inhibitions were calculated by subtracting the basal levels in the presence of VEGF from the individual treatments (Avastin® and BOL-303055-X) and the combinations. A custom test in the fit model platform (equivalent to the Scheffé's test) was used to determine whether the net inhibition of the combination was significantly different from the sum of the net inhibitions obtained in the individual treatments. A statistically significant difference showing that the combination is greater than the sum of individual treatment responses would indicate synergism, no significant differences would indicate additive effects, whereas significant differences below the sum of individual responses would indicate antagonistic effects. All the data from the individual wells were fitted to a re-parameterized four-parameter logistic equation to obtain IC₅₀ using similar methodology as that previously described and utilizing the statistical analysis software JMP (SAS Institute, Cary, N.C.).

### Results

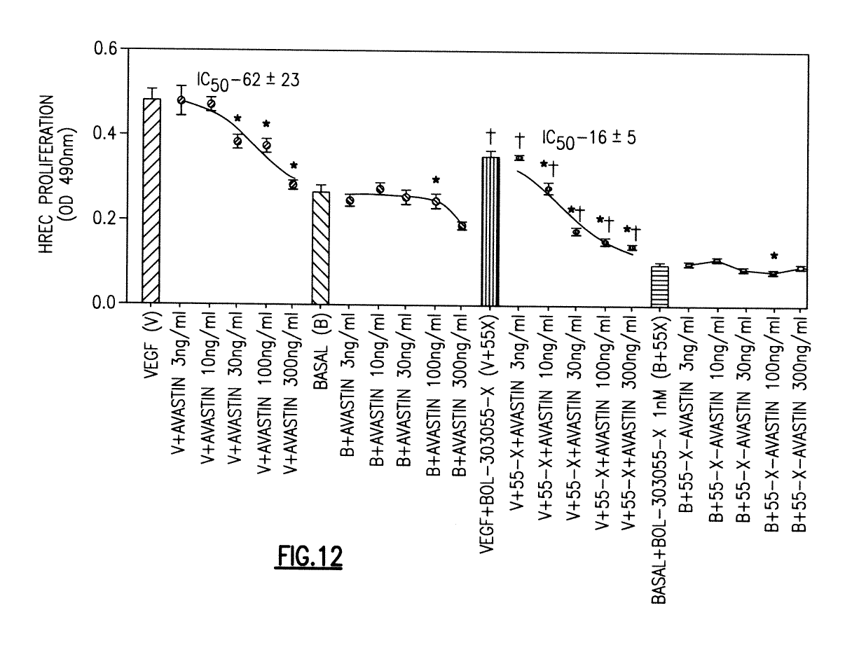

Under the stimulation of VEGF at 10 ng/ml, the proliferation of HREC was increased by approximately 1.5-fold compared to that of basal control and this difference was statistically significant (see FIG. 12). BOL-303055-X at 1 nM significantly inhibited HREC proliferation under basal conditions (see FIG. 12). Under basal conditions, Avastin® at 300 nM in the absence of BOL-303055-X, and at 100 nM in the presence of BOL-303055-X, showed a statistically significant reduction of HREC proliferation. These effects however were not dose-related and no effect at any other doses was observed (FIG. 12).

Avastin® from 3-300 ng/ml showed a dose-dependent inhibition on VEGF-induced HREC proliferation, with statistical significance starting from the 30 ng/ml dose (see FIG. 12). BOL-303055-X alone (1 nM) reduced VEGF-induced proliferation in a statistically significant manner when compared to the VEGF control (see FIG. 12). In the presence of BOL-303055-X, Avastin® from 3-300 ng/ml showed a dose-dependent reduction of VEGF-induced HREC proliferation, with statistical significance starting from 10 ng/ml dose (see FIG. 12). Furthermore, the inhibiting effect of Avastin at all doses was significantly greater in the presence of BOL-303055-X than in its absence (FIG. 12). Further calculations showed that the presence of BOL-303055-X at 1 nM shifted the IC₅₀ of Avastin® from 62 to 16 ng/ml. These data indicate that a low dose of BOL-303055-X enhances Avastin® potency by approximately 3.8-fold, suggesting a 3.8-fold improvement on the sensitivity of the cells to the antibody. Overall, these data clearly point out a beneficial effect of BOL-303055-X by enhancing Avastin® potency (FIG. 12, Table 13).

TABLE 13
Summary of Estimated IC₅₀ of Avastin ® Alone or Avastin ® Plus
BOL-303055-X on Inhibiting VEGF-Induced Proliferation of HREC

| | | 95% Conf. |
|---|---|---|
| Treatment | IC50 ± SE (ng/ml) | Limits |
| VEGF + Avastin | 62 ± 23 | 101-243 |
| VEGF + Avastin + BOL-303055-X | 16 ± 5 | 3.1-5.3 |

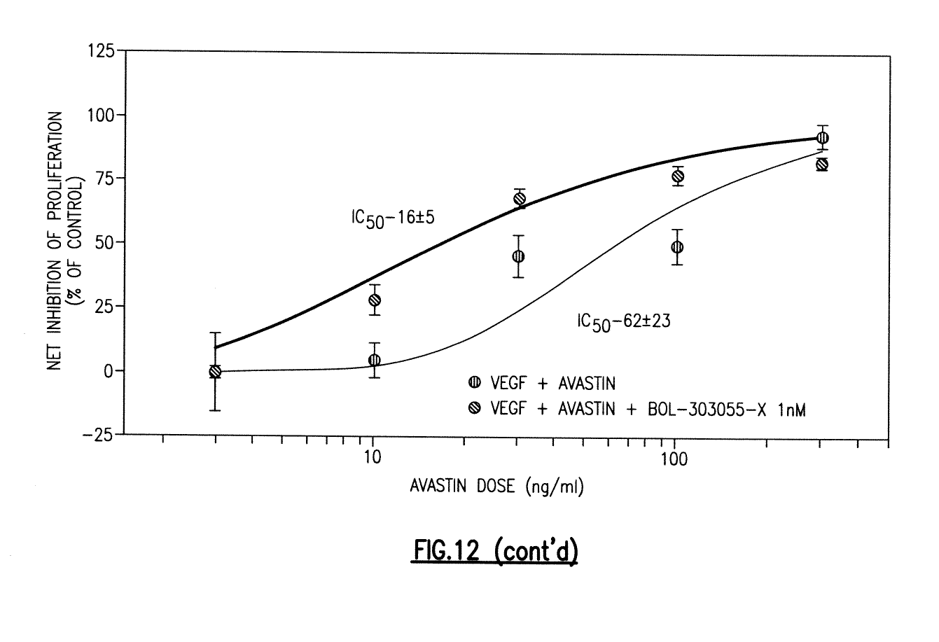

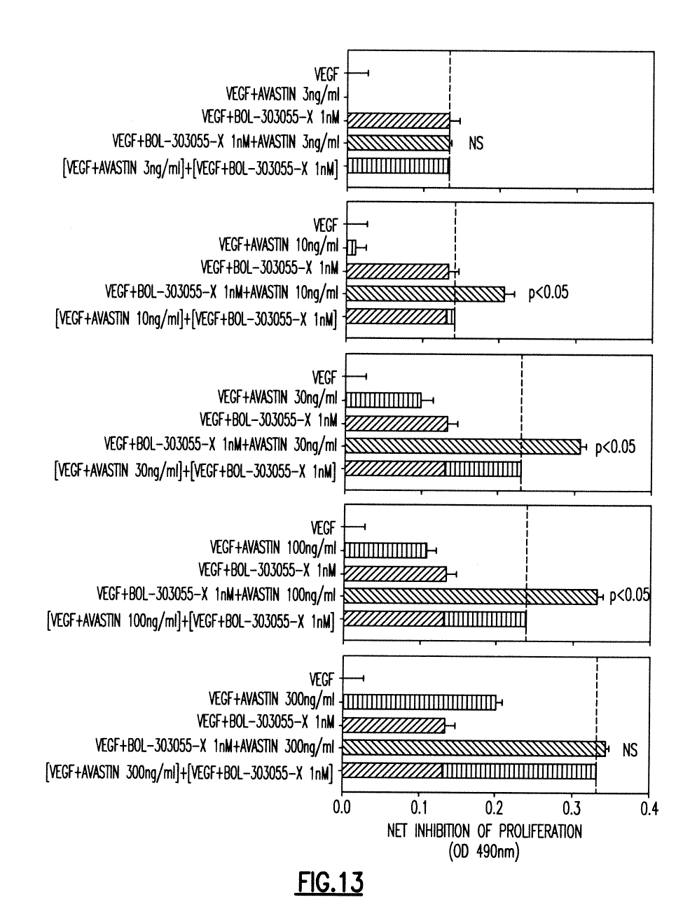

In order to assess whether the effects of BOL-303055-X and Avastin® were additive or synergistic, we calculated the net inhibition induced by either the integrin blockers or Avastin® alone and when given in combination. These data are presented in FIG. 13. From a theoretical point of view, additivity can be evaluated by analyzing whether the net effect of the combination is significantly greater than the sum of the individual responses. In FIG. 13, the last bar is a composite of the sum of individual responses and, therefore, represents the cutoff for additivity or synergy. Statistical evaluation using an analysis equivalent to the Scheffés test indicates that the inhibition of proliferation obtained by Avastin® from 10 to 100 ng/ml, when given in combination with BOL-303055-X, is significantly greater (P<0.05) than that of the sum of the individual responses to Avastin® and to BOL-303055-X alone. Therefore, these data indicate that BOL-303055-X exhibits synergistic effects with Avastin® to inhibit VEGF-induced proliferation of HREC.

### Conclusion

Results from this study demonstrate a synergist effect in the combination of BOL-303055-X and Avastin® on the inhibition of VEGF-induced proliferation of primary HREC. In addition, these data indicate that the addition of BOL-303055-X improves the potency of Avastin®, and, therefore, the sensitivity of the cells to the antibody, by approximately 3.8-fold.

Data indicate that at the very low end (3 ng/ml) and the very high end (300 ng/ml) additive effects were observed. The reasons why this is happening are different depending on the end of the dose-response curve. In the low end, Avastin® does not show activity, therefore, it is not possible to obtain more than the effects observed by BOL-303055-X. In contrast, at the high end the lack of synergy has to do with a ceiling effect which is innate to the design of this study and due the limitations of this in vitro model (FIG. 13).

One implication of this synergistic effect is that we could use lower doses or fewer injections of this integrin blocker or Avastin® if we combine these two agents together in treating neovascular diseases. This may result in reducing potential side effects of the drugs in addition to saving costs and potentially increasing efficacy. Alternatively, BOL-303055-X can enhance the sensitivity to anti-angiogenic therapy with Avastin® as observed in HREC.

### Testing 7: Study Evaluating the Effects of Integrin Antagonists and Lucentis Alone and in Combination on VEGF-Induced Proliferation of Human Retinal Endothelial Cells

### Purpose

To investigate the effects of integrin antagonists and Lucentis alone and in combination on VEGF-induced proliferation of HREC.

### Materials and Methods

### Design

HREC were first cultured overnight in a 96-well plate coated with fibronectin. The next day medium containing different agents as shown in Tables 14 and 15 was pre-incubated for 1 hour before being added into appropriate wells. The cells were cultured at 37° C., 5% CO₂, and 95% humidity for another 3 days. After that, cell proliferation was estimated by the MTS assay. In the first study we used VEGF 10 ng/mL (about EC₈₀ in study PH08169), Lucentis® 1 ng/mL (about EC₂₅ in study PH09038) and 5 doses of BOL-303055-X. Normal IgG (1 ng/ml) was also added to groups containing no Lucentis®. All groups contained the same amount of 0.1% DMSO. In the second study, all five compounds (BOL-303049-X, BOL-303050-X, BOL-303051-X, BOL-303054-X and BOL-303055-X) were tested at 1 nM with other conditions remaining the same.

TABLE 14
Experimental Design and Schedules - Study I

| | | Day 2 | |
|---|---|---|---|
| | | All groups were pre-incubated for | |
| Group* | Day 1 | 1 h before adding into cells | Day 5 |
| 1 | HREC | Basal (0.1% DMSO) | HREC |
| 2 | were | Basal + BOL-303055-X 0.1 nM | proliferation |
| 3 | cultured in | Basal + BOL-303055-X 0.3 nM | were |
| 4 | a 96-well | Basal + BOL-303055-X 1 nM | evaluated by |
| 5 | plate O/N | Basal + BOL-303055-X 3 nM | MTS assay |
| 6 | | Basal + BOL-303055-X 10 nM | |
| 7 | | Basal + Lucentis 1 ng/mL | |
| 8 | | Basal + Lucentis + | |
| | | BOL-303055-X 0.1 nM | |
| 9 | | Basal + Lucentis + | |
| | | BOL-303055-X 0.3 nM | |
| 10 | | Basal + Lucentis + | |
| | | BOL-303055-X 1 nM | |
| 11 | | Basal + Lucentis + | |
| | | BOL-303055-X 3 nM | |
| 12 | | Basal + Lucentis + | |
| | | BOL-303055-X 10 nM | |
| 13 | | VEGF 10 ng/mL | |
| 14 | | VEGF + BOL-303055-X 0.1 nM | |
| 15 | | VEGF + BOL-303055-X 0.3 nM | |
| 16 | | VEGF + BOL-303055-X 1 nM | |
| 17 | | VEGF + BOL-303055-X 3 nM | |
| 18 | | VEGF + BOL-303055-X 10 nM | |
| 19 | | VEGF + Lucentis + | |
| | | BOL-303055-X 0.1 nM | |
| 20 | | VEGF + Lucentis + | |
| | | BOL-303055-X 0.3 nM | |
| 21 | | VEGF + Lucentis + | |
| | | BOL-303055-X 1 nM | |
| 22 | | VEGF + Lucentis + | |
| | | BOL-303055-X 3 nM | |
| 23 | | VEGF + Lucentis + | |
| | | BOL-303055-X 10 nM | |

*Four wells per group

TABLE 15
Experimental Design and Schedule - Study II

| | | Day 2 | |
|---|---|---|---|
| | | All groups were pre-incubated for | |
| Group* | Day 1 | 1 h before adding into cells | Day 5 |
| 1 | HREC | Basal (0.1% DMSO) | HREC |
| 2 | were | Basal + BOL-303049-X 1 nM | proliferation |
| 3 | cultured in | Basal + BOL-303050-X 1 nM | were |
| 4 | a 96-well | Basal + BOL-303051-X 1 nM | evaluated by |
| 5 | plate O/N | Basal + BOL-303054-X 1 nM | MTS assay |
| 6 | | Basal + BOL-303055-X 1 nM | |
| 7 | | Basal + Lucentis 1 ng/mL | |
| 8 | | Basal + Lucentis + | |
| | | BOL-303049-X 1 nM | |
| 9 | | Basal + Lucentis + | |
| | | BOL-303050-X 1 nM | |
| 10 | | Basal + Lucentis + | |
| | | BOL-303051-X 1 nM | |
| 11 | | Basal + Lucentis + | |
| | | 0 BOL-303054-X 1 nM | |
| 12 | | Basal + Lucentis + | |
| | | BOL-303055-X 1 nM | |
| 13 | | VEGF 10 ng/mL | |
| 14 | | VEGF + BOL-303049-X 1 nM | |
| 15 | | VEGF + BOL-303050-X 1 nM | |
| 16 | | VEGF + BOL-303051-X 1 nM | |
| 17 | | VEGF + BOL-303054-X 1 nM | |
| 18 | | VEGF + BOL-303055-X 1 nM | |
| 19 | | VEGF + Lucentis + | |
| | | BOL-303049-X 1 nM | |
| 20 | | VEGF + Lucentis + | |
| | | BOL-303050-X 1 nM | |
| 21 | | VEGF + Lucentis + | |
| | | BOL-303051-X 1 nM | |
| 22 | | VEGF + Lucentis + | |
| | | BOL-303054-X 1 nM | |
| 23 | | VEGF + Lucentis + | |
| | | BOL-303055-X 1 nM | |

*4 wells per group

### Materials

Integrin antagonist compounds: BOL-303049-X, BOL-303050-X, BOL-303051-X, BOL-303054-X, AND BOL-303055-X

Compound stock solutions were made up in DMSO and stored at −20° C. To limit DMSO concentration to 0.1%, for every final incubation concentration, a 1000× working concentration was prepared in DMSO.

Human retinal endothelial cells (HREC) were purchased from Cell System (Kirkland, Wash.). The cells are routinely cultured and maintained in complete medium containing 10% FBS before experiment. Passages 4 were used.

### Other Reagents:

| | Catalog | | |
|---|---|---|---|
| Reagent | No. | Lot No. | Vendor |
| CS-C medium | 4Z0-500 | 47805 | Cell System (Kirkland, |
| | | | WA) |
| Human | F1904 | 0702053035 | Chemicon (Temecuia, CA) |
| fibronectin | | | |
| Dimethyl | 41639 | 399326/1 | Fluka (St. Louis, MO) |
| sulfoxide | | | |
| (DMSO) | | | |
| VEGF165 | 293- | 112208051 | R&D System |
| | VE/CF | | (Minneapolis, MN) |
| Lucentis ® | 8277704 | 759538 | Genentech (South San |
| | | | Francisco, CA) |

### Experiment

A 96-well plate was coated with human fibronectin (10 μg/mL) for 5 hours at 37° C. HREC were washed with HBSS buffer once, tripsinized and re-suspended in 10% FBS CS-C medium at 18,000 cells/mL. Cells (200 μL) were added to the plate and cultured overnight at 37° C., 5% CO₂, and 95% humidity. Next day, the cells were serum-starved for 2 hours.

For study I, VEGF at 10 ng/mL, Lucentis® at 1 ng/mL and BOL-303055-X at 0.1, 0.3, 1, 3 and 10 nM were prepared in 1% FBS CS medium as shown in Table 14. Normal IgG (1 ng/mL) were added to groups containing no Lucentis®. All medium solutions were incubated for 1 hour at 37° C. After carefully medium was aspirated, 200 μL of the medium containing different agents were then added into appropriate wells. The cells were cultured for another 3 days. For the study II as shown in the Table 15, five compounds were tested at 1 nM with other conditions remaining the same as study I.

At the end of incubation, the medium was carefully aspirated from cells and 120 μl of MTS working solution was added into all wells including blank wells (without cells). The plates were incubated at 37° C., 5% CO₂, and 95% humidity for 1 hour and then read by a plate reader at 490 nM wavelength.

### Data Analysis

Median optical density (OD) was generated from different groups. Data were expressed as mean±SEM of the OD values. Statistical analysis was performed using three-way ANOVA after Box-Cox transformations, and p<0.05 was considered statistically significant. To evaluate whether the effects of the combinations were either synergistic or additive, net inhibitions were calculated by subtracting the basal levels in the presence of VEGF from the individual treatments (Lucentis® and Integrin blockers) and the combinations. A custom test in the fit model platform (equivalent to the Scheffé's test) was used to determine whether the net inhibition of the combination was significantly different from the sum of the net inhibitions obtained in the individual treatments. A statistically significant difference showing that the combination was greater than the sum of net individual treatment responses would indicate synergism, no significant differences would indicate additive effects, whereas significant differences below the sum of individual responses would indicate antagonistic effects. Data analysis was performed utilizing the statistical analysis software JMP (SAS Institute, Cary, N.C.).

### Results

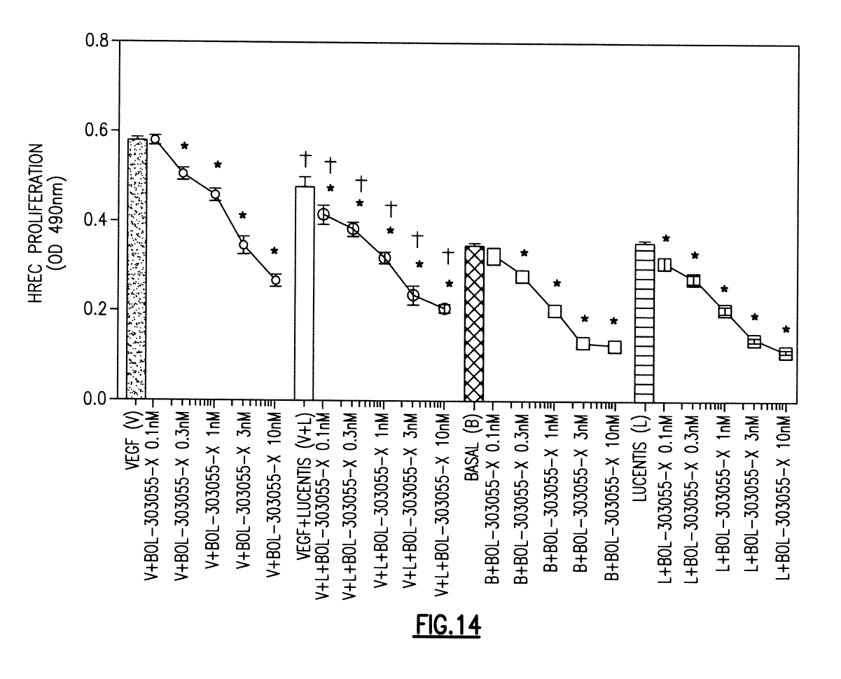

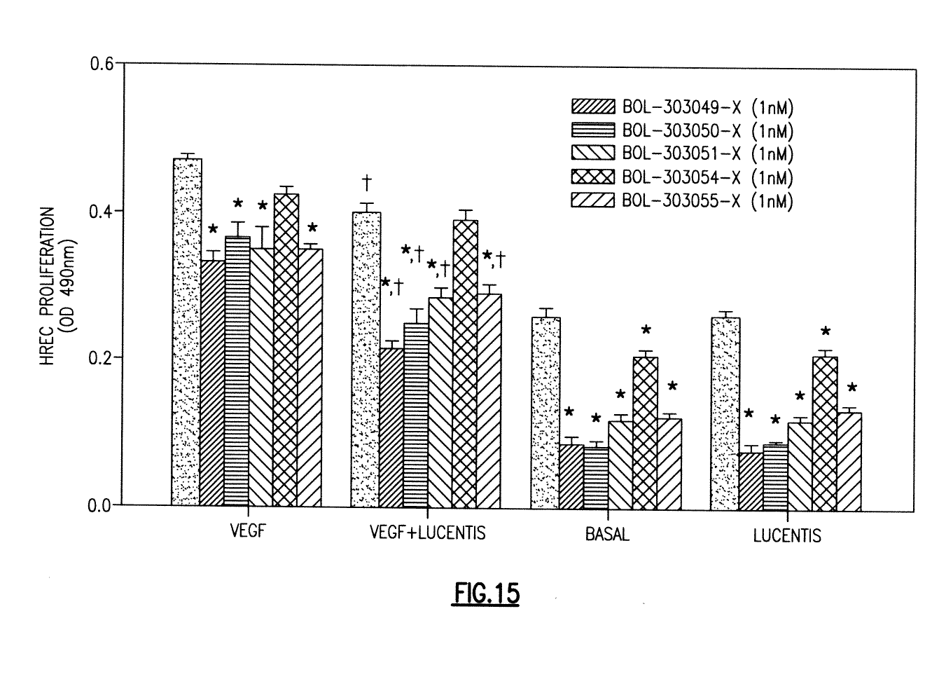

Under the stimulation of VEGF at 10 ng/mL, the proliferation of HREC was increased by approximately two-fold compared to that of basal control (see FIGS. 14 and 15). Lucentis® treatment alone did not alter HREC proliferation (see FIGS. 14 and 15). BOL-303055-X reduced HREC proliferation in a statistically significant and dose-dependent manner under basal and Lucentis®-treatment conditions (see FIG. 14). A similar statistically significant reduction in proliferation was observed when the compound was tested in study II (FIG. 15). In addition, the rest of the integrin blockers were also efficacious at reducing HREC proliferation both under basal and Lucentis®-treatment conditions (FIG. 15).

In the presence of Lucentis® at 1 ng/mL, VEGF-induced HREC proliferation was reduced by approximately 15-20% and this difference was statistically significant (see FIGS. 14 and 15). BOL-303055-X inhibited VEGF-induced HREC proliferation in a dose-dependent manner (FIG. 14, left hand bars and symbols). When tested in the presence of Lucentis®, BOL-303055-X at 0.1, 0.3, 1, 3 and 10 nM showed significantly greater inhibiting effect on VEGF-induced HREC proliferation than when tested without Lucentis® (compare symbols in the two left hand side sets of FIG. 14).

All 5 compounds at 1 nM except BOL-303054-X significantly inhibited VEGF-induced proliferation of HREC (FIG. 15).

When 5 integrin antagonists were used at 1 nM, the inhibiting effects of those compounds on VEGF-induced HREC proliferation was significantly greater in the presence of Lucentis® than in its absence for all the compounds except BOL-303054-X (FIG. 15).

The degrees of inhibition of all 5 compounds alone on HREC proliferation without VEGF stimulation observed here were similar to those observed in one of our previous studies (PH08110).

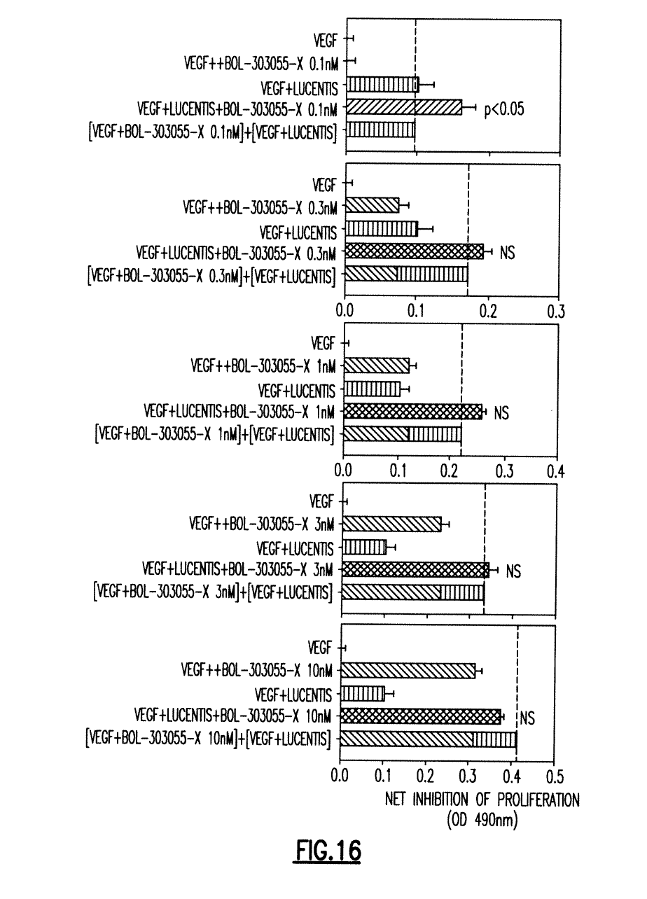

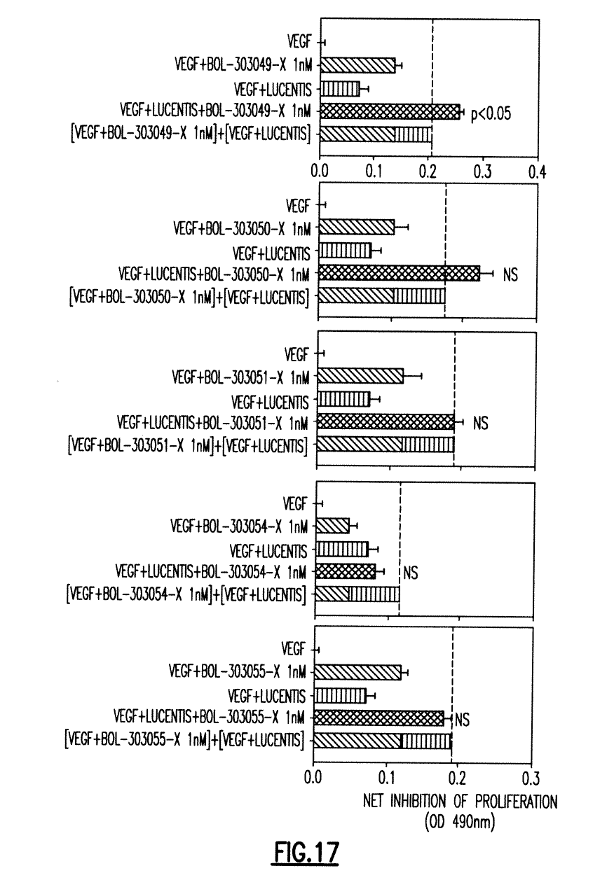

In order to assess whether the effects of the integrin blockers and Lucentis were additive or synergistic, we calculated the net inhibition induced by either the integrin blockers or Lucentis® alone and when given in combination. These data are presented in FIGS. 16 and 17. From a theoretical point of view, additivity or synergism can be evaluated by analyzing whether the net effect of the combination is significantly greater than the sum of the individual responses. In FIGS. 16 and 17, the last bar is a composite of the sum of individual responses and, therefore, represents the cutoff for additivity or synergy. Statistical analysis using an equivalent to the Scheffé's test indicate that the inhibition of proliferation obtained by BOL-303055-X in study I at 0.1 nM when given in combination with Lucentis is significantly greater (P<0.05) than that of the sum of the individual responses to the compound and to Lucentis® alone (see combo bar in top panel of FIG. 16). Therefore, these data indicate that the compound exhibits synergistic effects with Lucentis® to inhibit HREC proliferation. Interestingly, as the dose of the compound increases, the response is slightly blunted and by the next dose (0.3 nM) the effect of the combination is not statistically significant when compared to the sum of the individual net inhibitions for BOL-303055-X and Lucentis® (FIG. 16, second top panel). These data suggest that the effects at this dose are additive. Evaluation of the rest of the doses indicates that the 1, 3 and 10 nM doses of the compound behave similarly to the 0.3 nM dose, i.e., the effects of the combination at these concentrations are additive.

The same type of evaluation was conducted on the results of Study II. This analysis showed that the combination effects of Lucentis® with BOL-303049-X at 1 nM were synergistic, while the rest of four compounds at 1 nM were additive (FIG. 17). These data confirm those observed for BOL-303055-X in study I. There may be synergistic effects of integrin antagonists and Lucentis® at other (especially lower) concentrations.

### Conclusion

Results from this study suggest that there can be a synergistic effect in the combination of integrin antagonists and Lucentis® on the inhibition of VEGF-induced proliferation of primary human retinal endothelial cells. The data also indicate that the synergistic effects can only be observed at low doses of the integrin blockers, and this is probably due to a ceiling effect that occurs as a consequence of the experimental design of the study.

One implication of the possible synergistic/additive effect is that we could use lower doses or fewer injections of integrin antagonists or Lucentis® if we combine these two agents together in treating ocular neovascular diseases. This may result in reducing potential side effects of the drugs in addition to saving cost and potentially increase efficacy.

In still another aspect, a method for preparing a composition of the present invention comprises combining: (i) at least an integrin or vitronectin receptor antagonist, a prodrug thereof, or a pharmaceutically acceptable salt, ester, hydrate, solvate, or clathrate thereof; and (ii) a material selected from the group consisting of anti-inflammatory agents, anti-angiogenic agents, and combinations thereof; and (iii) a pharmaceutically acceptable carrier. In one embodiment, such a carrier can be a sterile saline solution or a physiologically acceptable buffer. In another embodiment, such a carrier comprises a hydrophobic medium, such as a pharmaceutically acceptable oil. In still another embodiment, such as carrier comprises an emulsion of a hydrophobic material and water.

In still another aspect, a method for preparing a composition of the present invention comprises combining: (i) at least an integrin or vitronectin receptor antagonist, or a pharmaceutically acceptable salt, ester, hydrate, or solvate thereof; and (ii) an anti-angiogenic agents; and (iii) a pharmaceutically acceptable carrier.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In certain embodiments, a composition of the present invention is formulated in a buffer having a slight acidic pH, such as from about 6 to about 6.8. In such embodiments, the buffer capacity of the composition desirably allows the composition to come rapidly to a physiological pH (about 7.4) after being administered to into the patient.

### Example 1

Two mixtures I and II are made separately by mixing the ingredients listed in Table E1. Five parts (by weight) of mixture I are mixed with twenty parts (by weight) of mixture II for 15 minutes or more. A preservative, such as PHMB, benzalkonium chloride, or polyquaternium-1, may be optionally added to the mixture to achieve a preservative concentration of about 0.001-0.03 percent (by weight). The pH of the combined mixture is adjusted to 6.2-6.8 using 1 N NaOH or 1 N HCl to yield a composition of the present invention.

TABLE E1

| Ingredient | Amount | |
|---|---|---|
| Mixture I | | |
| Carbopol 934P NF | 0.25 | g |
| Purified water | 99.75 | g |
| Mixture II | | |
| Propylene glycol | 5 | g |
| EDTA | 0.1 | mg |
| Compound of Formula IV | 0.5 | g |

### Example 2

Two mixtures I and II are made separately by mixing the ingredients listed in Table E2. Five parts (by weight) of mixture I are mixed with twenty parts (by weight) of mixture II for 15 minutes or more. A preservative, such as PHMB or polyquaternium-1 (also known as Polyquad®), may be optionally added to the mixture to achieve a preservative concentration of about 0.001-0.03 percent (by weight). The pH of the combined mixture is adjusted to 6.6-7.2 using 1 N NaOH or 1N HCl to yield a composition of the present invention.

TABLE E2

| Ingredient | Amount | |
|---|---|---|
| Mixture I | | |
| Lucentis ® | 0.5 | g |
| Carbopol 934P NF | 0.25 | g |
| Purified water | 99.25 | g |
| Mixture II | | |
| Propylene glycol | 5 | g |
| EDTA | 0.1 | mg |
| Compound of Formula IV | 0.5 | g |

### Example 3

Two mixtures I and II are made separately by mixing the ingredients listed in Table E3. Five parts (by weight) of mixture I are mixed with twenty parts (by weight) of mixture II for 15 minutes or more. The pH of the combined mixture is adjusted to 6.2-6.4 using 1 N NaOH or 1 N HCl to yield a composition of the present invention.

TABLE E3

| Ingredient | Amount | |
|---|---|---|
| Mixture I | | |
| Lucentis ® | 0.2 | g |
| Carbopol 934P NF | 0.25 | g |
| Purified water | 99.55 | g |
| Mixture II | | |
| Propylene glycol | 3 | g |
| Triacetin | 7 | g |
| Compound of Formula III | 0.75 | g |
| EDTA | 0.1 | mg |

### Example 4

Two mixtures I and II are made separately by mixing the ingredients listed in Table E4. Five parts (by weight) of mixture I are mixed with twenty parts (by weight) of mixture II for 15 minutes or more. The pH of the combined mixture is adjusted to 6.2-6.4 using 1 N NaOH or 1 N HCl to yield a composition of the present invention.

TABLE E4

| Ingredient | Amount | |
|---|---|---|
| Mixture I | | |
| Avastin ® | 0.3 | g |
| N-isoxazol-3-yl-4-(2-phenyl-1H- | 0.3 | g |
| imidazol-1-yl)pyrimidin-2-amine (a | | |
| tyrosine kinase inhibitor) | | |
| Carbopol 934P NF | 0.25 | g |
| Olive oil | 99.15 | g |
| Mixture II | | |
| Propylene glycol | 7 | g |
| Glycerin | 3 | g |
| Compound of Formula III | 0.2 | g |
| Cyclosporine A | 5 | g |
| HAP (30%) | 0.5 | mg |
| Alexidine 2HCl | 1-2 | ppm |

Note:
“HAP” denotes hydroxyalkyl phosphonates, such as those known under the trade name Dequest ®.

### Example 5

The ingredients listed in Table E5 are mixed together for at least 15 minutes. The pH of the mixture is adjusted to 6.4-7 using 1 N NaOH or 1N HCl to yield a composition of the present invention.

TABLE E5

| Ingredient | Amount | |
|---|---|---|
| Povidone | 1 | g |
| HAP (30%) | 0.05 | g |
| Glycerin | 3 | g |
| Propylene glycol | 3 | g |
| Compound of Formula IV | 0.5 | g |
| Lucentis ® | 0.1 | g |
| Tyloxapol | 0.25 | g |
| Benzalkonium chloride (“BAK”) | 10-100 | ppm |
| Purified water | q.s. to 100 | g |

### Example 6

The ingredients listed in Table E6 are mixed together for at least 15 minutes. The pH of the mixture is adjusted to 6.2-6.4 using 1 N NaOH or 1 N HCl to yield a composition of the present invention.

TABLE E6

| Ingredient | Amount | |
|---|---|---|
| Povidone | 1.5 | g |
| HAP (30%) | 0.05 | g |
| Glycerin | 3 | g |
| Propylene glycol | 3 | g |
| Compound of Formula IV | 0.75 | g |
| VEGF ribozyme | 0.1 | g |
| Tyloxapol | 0.25 | g |
| Alexidine 2HCl | 1-2 | ppm |
| Purified water | q.s. to 100 | g |

### Example 7

The ingredients listed in Table E7 are mixed together for at least 15 minutes. The pH of the mixture is adjusted to 6.2-6.4 using 1 N NaOH or 1N HCl to yield a composition of the present invention.

TABLE E7

| Ingredient | Amount | |
|---|---|---|
| CMC (MV) | 0.5 | g |
| HAP (30%) | 0.05 | g |
| Glycerin | 3 | g |
| Propylene glycol | 3 | g |
| Compound of Formula IV | 0.75 | g |
| Macugen ® | 0.1 | g |
| N-(3-methylisoxazol-5-yl)-4-(2-phenyl- | 0.3 | g |
| 1H-imidazol-1-yl)pyrimidin-2-amine (a | | |
| tyrosine kinase inhibitor) | | |
| Tyloxapol (a surfactant) | 0.25 | g |
| Alexidine 2HCl | 1-2 | ppm |

| | |
|---|---|
| Sunflower oil | q.s. to 100 |

### Example 8

The ingredients listed in Table E8 are mixed together for at least 15 minutes. The pH of the mixture is adjusted to 6.2-6.8 using 1 N NaOH or 1N HCl to yield a composition of the present invention.

TABLE E8

| Ingredient | Amount | |
|---|---|---|
| CMC (MV) | 0.5 | g |
| HAP (30%) | 0.05 | g |
| Glycerin | 3 | g |
| Propylene glycol | 3 | g |
| Compound of Formula IV | 0.75 | g |
| dsRNA having sequence corresponding to | 0.2 | g |
| single-stranded VEGF mRNA | | |
| N-(3-methylisoxazol-5-yl)-4-(2-phenyl- | 0.3 | g |
| 1H-imidazol-1-yl)pyrimidin-2-amine (a | | |
| tyrosine kinase inhibitor) | | |
| Tyloxapol (a surfactant) | 0.25 | g |
| Alexidine 2HCl | 1-2 | ppm |

| | |
|---|---|
| Purified water | q.s. to 100 |

### Example 9

The ingredients listed in Table E9 are mixed together for at least 15 minutes. The pH of the mixture is adjusted to 6.2-6.4 using 1 N NaOH or 1 N HCl to yield a composition of the present invention.

TABLE E9

| Ingredient | Amount | |
|---|---|---|
| CMC (MV) | 0.5 | g |
| HAP (30%) | 0.05 | g |
| Glycerin | 3 | g |
| Propylene glycol | 3 | g |
| Compound of Formula IX | 0.75 | g |
| Lucentis ® | 0.2 | g |
| dsRNA having sequence corresponding to | 0.2 | g |
| single-stranded VEGF mRNA | | |
| N-(3-methylisoxazol-5-yl)-4-(2-phenyl- | 0.3 | g |
| 1H-imidazol-1-yl)pyrimidin-2-amine (a | | |
| tyrosine kinase inhibitor) | | |
| Tyloxapol (a surfactant) | 0.25 | g |
| Alexidine 2HCl | 1-2 | ppm |

| | |
|---|---|
| Corn oil | q.s. to 100 |

### Example 10

The ingredients listed in Table E10 are mixed together for at least 15 minutes. The pH of the mixture is adjusted to 6.4-7 using 1 N NaOH or 1 N HCl to yield a composition of the present invention.

TABLE E10

| Ingredient | Amount | |
|---|---|---|
| CMC (MV) | 0.5 | g |
| HAP (30%) | 0.05 | g |
| Glycerin | 3 | g |
| PEG-400 | 3 | g |
| Compound of Formula IV | 0.75 | g |
| Macugen ® | 0.2 | g |
| Polypeptide antibody against VEGFR-2 | 0.3 | g |
| N-(3-methylisoxazol-5-yl)-4-(2-phenyl- | 0.3 | g |
| 1H-imidazol-1-yl)pyrimidin-2-amine (a | | |
| tyrosine kinase inhibitor) | | |
| Tyloxapol (a surfactant) | 0.25 | g |
| Alexidine 2HCl | 1-2 | ppm |
| Purified water | q.s. to 100 | g |

### Example 11

The ingredients listed in Table E11 are mixed together for at least 15 minutes. The pH of the mixture is adjusted to 6.6-7 using 1 N NaOH or 1N HCl to yield a composition of the present invention.

TABLE E11

| Ingredient | Amount | |
|---|---|---|
| Povidone | 1 | g |
| HAP (30%) | 0.05 | g |
| Glycerin | 3 | g |
| Propylene glycol | 3 | g |
| Compound of Formula IV | 0.5 | g |
| Avastin ® | 0.1 | g |
| Tyloxapol | 0.25 | g |
| Polyquaternium-1 | 10-50 | ppm |
| Purified water | q.s. to 100 | g |

In another aspect, an integrin or vitronectin receptor antagonist as disclosed herein, a prodrug thereof, or a pharmaceutically acceptable salt, ester, hydrate, or solvate thereof is incorporated into a formulation for topical administration, systemic administration, periocular injection, or intravitreal injection. Such a formulation can also comprise a material selected from the group consisting of anti-inflammatory agents (such as those disclosed herein), VEGF (in particular, VEGF-A) antagonists, and combinations thereof. An injectable intravitreal formulation can desirably comprise a carrier that provides a sustained-release of the active ingredients, such as for a period longer than about 1 week (or longer than about 1, 2, 3, 4, 5, or 6 months). In certain embodiments, the sustained-release formulation desirably comprises a carrier that is insoluble or only sparingly soluble in the vitreous. Such a carrier can be an oil-based liquid, emulsion, gel, or semisolid. Non-limiting examples of oil-based liquids include castor oil, peanut oil, olive oil, coconut oil, sesame oil, cottonseed oil, corn oil, sunflower oil, fish-liver oil, arachis oil, and liquid paraffin.

In one embodiment, a compound or composition of the present invention can be injected intravitreally, for example through the pars plana of the ciliary body, to treat or prevent glaucoma or progression thereof using a fine-gauge needle, such as 25-30 gauge. Typically, an amount from about 25 μl to about 100 μl of a composition comprising an integrin or vitronectin receptor antagonist of the present invention, a prodrug thereof, or a pharmaceutically acceptable salt, ester, hydrate, or solvate thereof is administered into a patient. A concentration of such integrin or vitronectin receptor antagonist, prodrug thereof, or pharmaceutically acceptable salt, ester, hydrate, or solvate thereof is selected from the ranges disclosed above.

In another aspect, an integrin or vitronectin receptor antagonist of the present invention, a prodrug thereof, or a pharmaceutically acceptable salt, ester, hydrate, solvate, or clathrate thereof is incorporated into an ophthalmic device that comprises a biodegradable material, and the device is implanted into a subject to provide a long-term (e.g., longer than about 1 week, or longer than about 1, 2, 3, 4, 5, or 6 months) treatment of the chronic inflammatory condition. Such a device may be implanted by a skilled physician in the subject's ocular or periocular tissue.

In still another aspect, a method for treating, reducing, ameliorating, alleviating, or inhibiting the progression of, pathological ocular neovascularization comprises: (a) providing a composition comprising an integrin or vitronectin receptor antagonist, a prodrug thereof, or a pharmaceutically acceptable salt, ester, hydrate, solvate, enantiomer, or polymorph thereof; and (b) administering to a subject an amount of the composition at a frequency sufficient to treat, reduce, ameliorate, or alleviate the condition or disorder in the subject.

In one embodiment, the integrin or vitronectin receptor antagonist is selected from among those disclosed above. In another embodiment, the integrin or vitronectin receptor antagonist is selected from the group consisting of compounds having Formulae I-XI, pharmaceutically acceptable salts, esters, hydrates, solvates, enantiomers, and polymorphs thereof, and mixtures thereof. In still another embodiment, the integrin or vitronectin receptor antagonist is selected from the group consisting of compounds having Formulae II-X, pharmaceutically acceptable salts, esters, hydrates, solvates, enantiomers, and polymorphs thereof, and mixtures thereof.

In another aspect, the composition further comprises a VEGF-A inhibitor. In one embodiment, the VEGF-A inhibitor comprises bevacizumab (also known as Avastin®). In another embodiment, the VEGF-A inhibitor comprises ranibizumab (also known as Lucentis®).

In another embodiment, such pathological ocular neovascularization results from a chronic inflammation. In one embodiment, such chronic inflammation comprises inflammation of a vasculature. In another embodiment, such chronic inflammation comprises inflammation of the ocular vasculature.

In still another embodiment, such pathological ocular neovascularization results in or comprises a condition or disorder is selected from the group consisting of DR, AMD, DME, posterior uveitis, corneal neovascularization, iris neovascularization, neovascularization of the retina, neovascularization of the choroid, and combinations thereof.

In another embodiment, the composition further comprises: (i) an anti-inflammatory agent, a prodrug thereof, or a pharmaceutically acceptable salt or ester thereof; (ii) an anti-angiogenic agent; or (iii) a combination thereof. Such an anti-inflammatory agent or anti-angiogenic agent is selected from among those disclosed above. The concentration of the integrin or vitronectin receptor antagonist, a prodrug thereof, or a pharmaceutically acceptable salt, ester, hydrate, or solvate thereof, the anti-inflammatory agent or anti-angiogenic agent is selected from among the ranges disclosed above.

In another aspect, an integrin or vitronectin receptor antagonist, a prodrug thereof, or a pharmaceutically acceptable salt, ester, hydrate, or solvate thereof, with or without an additional anti-inflammatory agent and/or an anti-angiogenic agent, is incorporated into a formulation for topical administration, systemic administration, periocular injection, or intravitreal injection. An injectable intravitreal formulation can desirably comprise a carrier that provides a sustained-release of the active ingredients, such as for a period longer than about 1 week (or longer than about 1, 2, 3, 4, 5, or 6 months).

In still another aspect, an integrin or vitronectin receptor antagonist, a prodrug thereof, or a pharmaceutically acceptable salt, ester, hydrate, or solvate thereof is incorporated into an ophthalmic device that comprises a biodegradable material, and the device is implanted into a subject to provide a long-term (e.g., longer than about 1 week, or longer than about 1, 2, 3, 4, 5, or 6 months) treatment of a back-of-the-eye disease. Such a device may be implanted by a skilled physician in the back of the eye of the patient for the sustained release of the active ingredient or ingredients. A typical implant system or device suitable for use in a method of the present invention comprises a biodegradable matrix with the active ingredient or ingredients impregnated or dispersed therein. Non-limiting examples of ophthalmic implant systems or devices for the sustained-release of an active ingredient are disclosed in U.S. Pat. Nos. 5,378,475; 5,773,019; 5,902,598; 6,001,386; 6,051,576; and 6,726,918; which are incorporated herein by reference in their entireties.

In yet another aspect, a composition of the present invention is administered once a week, once a month, once a year, twice a year, four times a year, or at a suitable frequency that is determined to be appropriate by a skilled medical practitioner for treating, reducing, ameliorating, alleviating, or inhibiting the progression of, the condition or disorder.

In a further aspect, the present invention provides a method for treating, reducing, ameliorating, alleviating, or inhibiting the progression of, pathological ocular neovascularization (or angiogenesis) that has an etiology in inflammation (in particular, chronic inflammation). The method comprises: (a) administering an amount of a composition comprising an integrin or vitronectin receptor antagonist, a prodrug thereof, or a pharmaceutically acceptable salt, ester, hydrate, or solvate thereof to a subject at a first frequency sufficient to treat, reduce, ameliorate, alleviate, or inhibit the progression of, said pathological ocular neovascularization in the subject; and (b) performing a procedure selected from the group consisting of protocoagulation, photodynamic therapy, and a combination thereof in the subject at a second frequency sufficient to treat, reduce, ameliorate, alleviate, or inhibit the progression of, said pathological ocular revascularization in the subject. In one embodiment, the composition further comprises an anti-inflammatory agent, an anti-angiogenic agent, or a combination thereof. Non-limiting examples of these materials, and their suitable concentrations in the composition are disclosed herein above.

In one embodiment, the first frequency and the second frequency are the same. In another embodiment, the first frequency and the second frequency are different. In still another embodiment, said administering and said performing are carried out sequentially. In yet another embodiment, said performing is carried out before said administering. In a further embodiment, said performing is carried out after said administering. The first frequency and the second frequency can be, for example, once a week, once a month, once a year, twice a year, four times a year, or other frequencies, said first frequency and second frequency being chosen as deemed appropriate for the condition and treatment objective.

In photocoagulation therapy, high-energy light from a laser is directed to the leaky vasculature to coagulate the fluid in and around the new leaky vessels, relying on the transfer of thermal energy generated by the laser to the pathological tissue. Photocoagulation systems are currently available.

In photodynamic therapy (“PDT”), a photosensitizer (light-activated drug) is administered into the patient, typically via the intravenous route followed by application of light of appropriate wavelength directed at the pathological tissue, such as the leaky vasculature. The light sources most commonly used are non-thermal lasers or light-emitting diodes (“LEDs”). After exposure to light at a wavelength absorbed by the photosensitizer, an energy transfer cascade is initiated, culminating in the formation of reactive oxygen, which generates free radicals. These free radicals, in turn, disrupt cellular structures or functions, leading to death of endothelial cells and, thus, prevention of further neovascularization. Non-limiting examples of photosensitizers and methods for PDT include those disclosed in U.S. Pat. Nos. 7,015,240 and 7,060,695; which are incorporated herein by reference.

### Comparison of Glucocorticoids and Integrin or Vitronection Receptor Antagonists of the Present Invention

One of the most frequent undesirable actions of a glucocorticoid therapy is steroid diabetes. The reason for this undesirable condition is the stimulation of gluconeogenesis in the liver by the induction of the transcription of hepatic enzymes involved in gluconeogenesis and metabolism of free amino acids that are produced from the degradation of proteins (catabolic action of glucocorticoids). A key enzyme of the catabolic metabolism in the liver is the tyrosine aminotransferase (“TAT”). The activity of this enzyme can be determined photometrically from cell cultures of treated rat hepatoma cells. Thus, the gluconeogenesis by a glucocorticoid can be compared to that of an integrin or vitronectin receptor antagonist of the present invention by measuring the activity of this enzyme. For example, in one procedure, the cells are treated for 24 hours with the test substance (an integrin or vitronectin receptor antagonist of the present invention or glucocorticoid), and then the TAT activity is measured. The TAT activities for the selected an integrin or vitronectin receptor antagonist of the present invention and glucocorticoid are then compared. Other hepatic enzymes can be used in place of TAT, such as phosphoenolpyruvate carboxykinase, glucose-6-phosphatase, or fructose-2,6-biphosphatase. Alternatively, the levels of blood glucose in an animal model may be measured directly and compared for individual subjects that are treated with a glucocorticoid for a selected condition and those that are treated with an integrin or vitronectin receptor antagonist of the present invention for the same condition.

Another undesirable result of glucocorticoid therapy is GC-induced cataract. The cataractogenic potential of a compound or composition may be determined by quantifying the effect of the compound or composition on the flux of potassium ions through the membrane of lens cells (such as mammalian lens epithelial cells) in vitro. Such an ion flux may be determined by, for example, electrophysiological techniques or ion-flux imaging techniques (such as with the use of fluorescent dyes). An exemplary in-vitro method for determining the cataractogenic potential of a compound or composition is disclosed in U.S. Patent Application Publication 2004/0219512, which is incorporated herein by reference.

Still another undesirable result of glucocorticoid therapy is hypertension. Blood pressure of similarly matched subjects treated with a glucocorticoid or an integrin or vitronectin receptor antagonist of the present invention for a pathological neovascularization condition may be measured directly and compared.

Yet another undesirable result of glucocorticoid therapy is increased IOP. IOP of similarly matched subjects treated with a glucocorticoid or an integrin or vitronectin receptor antagonist of the present invention for a pathological neovascularization condition may be measured directly and compared.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition for treating, reducing, ameliorating, or inhibiting progression of, pathological ocular neovascularization in a subject, the composition comprising: (a) an integrin or vitronectin receptor antagonist having Formula I 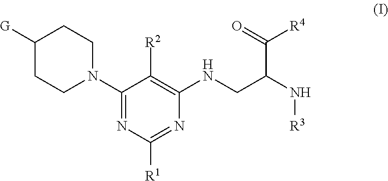
wherein G represents a substituted or unsubstituted aryl or heteroaryl group; substituted or unsubstituted heterocycle group; R⁷R⁸N—C(═NR⁶)—NH—CO-A-NH—CO—; A-NH—CH₂—; wherein A represents an aryl, heteroaryl, or heterocycle group, unsubstituted or substituted with one or more R⁹ groups;
R¹ represents a hydrogen atom; a halogen atom, a nitro group; (C₁-C₄)alkyl-; (C₁-C₄)alkoxy-; (C₅-C₁₄)Ar—; (C₅-C₁₄)Ar—(C₁-C₄)alkyl- group; an amino radical unsubstituted or monosubstituted or disubstituted with an alkyl radical and/or an acyl radical containing 1 to 4 carbon atoms, a —(CH₂)₀₋₂—C(O)OR⁵ group; or a —(CH₂)₀₋₂—OR⁵ group;
R² represents a hydrogen atom; a halogen atom; a nitro group; an alkyl radical containing 1 to 4 carbon atoms; (C₁-C₄)alkoxy-; an amino radical unsubstituted or monosubstituted or disubstituted with an alkyl and/or an acyl containing 1 to 4 carbon atoms; a —(CH₂)₀₋₂—C(O)OR⁵ group; or a —(CH₂)₀₋₂—OR⁵ group;
R³ represents a hydrogen atom, a —C(O)OR⁵ radical, an —SO₂R⁵ radical, or a monocyclic or polycyclic system comprising a 4- to 10-membered aromatic or non-aromatic ring system, the ring or at least one of the rings containing 1 to 4 heteroatoms chosen from N, O or S, unsubstituted or substituted with one or more R⁹ radicals;
R⁴ represents OH; (C₁-C₈)-alkoxy-; (C₅-C₁₄)—Ar—(C₁-C₄)-alkoxy-; (C₅-C₁₄)-aryloxy-; (C₃-C₁₂)-cycloalkyloxy; (C₃-C₁₂)-cycloalkyl-(C₁-C₄)-alkyloxy-; (C₁-C₈)-alkyl-carbonyloxy-(C₁-C₄)-alkyloxy-; (C₅-C₁₄)—Ar—(C₁-C₄)-alkyl-carbonyloxy-(C₁-C₄)-alkyloxy-; (C₁-C₈)dialkylaminocarbonylmethyloxy-; (C₅-C₁₄)—Ar—(C₁-C₄)-dialkylaminocarbonylmethyloxy-; an amino radical unsubstituted or monosubstituted or disubstituted with a (C₁-C₄)-alkyl and/or (C₅-C₁₄)—Ar and/or (C₅-C₁₄)—Ar—(C₁-C₄)-alkyl- radical and/or a (C₁-C₅)-acyl radical; or the remainder of aspartic acid (D) or leucine (L);
R⁵ represents (C₁-C₈)-alkyl-; (C₁-C₈)-alkyl-C(O)O—(C₁-C₈)-alkyl-; (C₁-C₈)-alkyl-S(O)(O)—(C₁-C₈)-alkyl-; (C₅-C₁₄)—Ar—; (C₅-C₁₄)—Ar—(C₁-C₄)-alkyl-; (C₅-C₁₄)—Ar—C(O)O—(C₁-C₄)-alkyl-; (C₃-C₁₂)-cycloalkyl-; (C₃-C₁₂)-cycloalkyl-(C₁-C₄)-alkyl-; bicycloalkyl-(C₁-C₄)-alkyl-; tricycloalkyl-(C₁-C₄)-alkyl-; said Ar, alkyl, cycloalkyl, bicycloalkyl and tricycloalkyl radicals being unsubstituted or substituted by one or more R⁹ groups;
R⁶ represents a hydrogen atom; a hydroxyl; nitro; (C₁-C₆)-alkyl-O—C(O)—; (C₁-C₆)-alkyl-O—C(O)O— group;
R⁷ and R⁸, independently of one another represent a hydrogen atom or a (C₁-C₆)-alkyl radical unsubstituted or substituted with R⁹;
R⁹ represents halogen; amino; nitro ;hydroxyl; (C₁-C₄)alkoxy; (C₁-C4)alkylthio; carboxy; (C₁-C₄)alkyloxycarbonyl; (C₁-C₈)alkyl unsubstituted or substituted with one or more halogen atoms; (C₅-C₁₄)Ar; (C₅-C14)Ar—(C₁-C₄)alkyl;
or one or more isomers of a compound having Formula I, alone or in a mixture, a pharmaceutically acceptable salt, ester, hydrate, solvate, clathrate, enantiomer, or polymorph thereof; and (b) a VEGF-A inhibitor.

2. The composition of claim 1, wherein the VEGF-A inhibitor comprises an anti-VEGF-A antibody.

3. The composition of claim 2, wherein the VEGF-A inhibitor comprises bevacizumab, ranibizumab, or a combination thereof.

4. The composition of claim 2, wherein the VEGF-A inhibitor comprises a VEGF-A aptamer.

5. The composition of claim 2, further comprising an anti-inflammatory agent.

6. The composition of claim 5, wherein the anti-inflammatory agent comprises a material selected from the group consisting of NSAIDs, PPAR ligands, combinations thereof, and mixtures thereof.

7. The composition of claim 1, wherein the integrin or vitronectin receptor antagonist and the VEGF-A inhibitor are present together in the composition in amounts sufficient to be effective for treating, reducing, ameliorating, alleviating, or inhibiting progression of, pathological ocular neovascularization.

8. The composition of claim 7, wherein the pathological ocular neovascularization has an etiology in inflammation.

9. The composition of claim 9, wherein the pathological ocular neovascularization is selected from the group consisting of diabetic retinopathy (“DR”), dry age-related macular degeneration (“AMD”), wet AMD, diabetic macular edema (“DME”), retinal detachment, posterior uveitis, corneal neovascularization, iris neovascularization, and combinations thereof.

10. The composition of claim 7, wherein the pathological ocular neovascularization is wet AMD.

11. The composition of claim 7, wherein the pathological ocular neovascularization is diabetic retinopathy.

12. The composition of claim 7, wherein the composition comprises liquid medium.

13. The composition of claim 7, wherein the composition comprises an ophthalmic device.

14. The composition of claim 1, wherein the integrin or vitronectin receptor antagonist comprises a compound having any one of Formulae II-X, or a free acid thereof, or a pharmaceutically acceptable salt, ester, hydrate, solvate, enantiomer, or polymorph thereof.

15. The composition of claim 1, wherein the integrin or vitronectin receptor antagonist comprises a compound having Formula IV, or a pharmaceutically acceptable salt, ester, hydrate, solvate, enantiomer, or polymorph thereof.

16. A method for treating, reducing, ameliorating, or inhibiting progression of, pathological ocular neovascularization in a subject, the method comprising administering to an ocular environment of an affected eye of the subject a composition that comprises an integrin or vitronectin receptor antagonist having Formula I 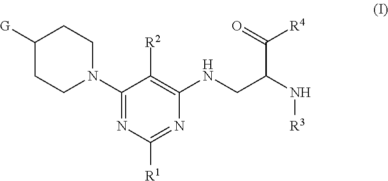
wherein G represents a substituted or unsubstituted aryl or heteroaryl group; substituted or unsubstituted heterocycle group; R⁷R⁸N—C(═NR⁶)—NH—CO-A-NH—CO—; A-NH—CH₂—; wherein A represents an aryl, heteroaryl, or heterocycle group, unsubstituted or substituted with one or more R⁹ groups;
R¹ represents a hydrogen atom; a halogen atom, a nitro group; (C₁-C₄)alkyl-; (C₁-C₄)alkoxy-; (C₅-C₁₄)Ar—; (C₅-C₁₄)Ar—(C₁-C₄)alkyl- group; an amino radical unsubstituted or monosubstituted or disubstituted with an alkyl radical and/or an acyl radical containing 1 to 4 carbon atoms, a —(CH₂)₀₋₂—C(O)OR⁵ group; or a —(CH₂)₀₋₂—OR⁵ group;
R² represents a hydrogen atom; a halogen atom; a nitro group; an alkyl radical containing 1 to 4 carbon atoms; (C₁-C₄)alkoxy-; an amino radical unsubstituted or monosubstituted or disubstituted with an alkyl and/or an acyl containing 1 to 4 carbon atoms; a —(CH₂)₀₋₂—C(O)OR⁵ group; or a —(CH₂)₀₋₂—OR⁵ group;
R³ represents a hydrogen atom, a —C(O)OR⁵ radical, an —SO₂R⁵ radical, or a monocyclic or polycyclic system comprising a 4- to 10-membered aromatic or non-aromatic ring system, the ring or at least one of the rings containing 1 to 4 heteroatoms chosen from N, O or S, unsubstituted or substituted with one or more R⁹ radicals;
R⁴ represents OH; (C₁-C₈)-alkoxy-; (C₅-C₁₄)—Ar—(C₁-C₄)-alkoxy-; (C₅-C₁₄)-aryloxy-; (C₃-C₁₂)-cycloalkyloxy; (C₃-C₁₂)-cycloalkyl-(C₁-C₄)-alkyloxy-; (C₁-C₈)-alkyl-carbonyloxy-(C₁-C₄)-alkyloxy-; (C₅-C₁₄)—Ar—(C₁-C₄)-alkyl-carbonyloxy-(C₁-C₄)-alkyloxy-; (C₁-C₈)dialkylaminocarbonylmethyloxy-; (C₅-C₁₄)—Ar—(C₁-C₄)-dialkylaminocarbonylmethyloxy-; an amino radical unsubstituted or monosubstituted or disubstituted with a (C₁-C₄)-alkyl and/or (C₅-C₁₄)—Ar and/or (C₅-C₁₄)—Ar—(C₁-C₄)-alkyl- radical and/or a (C₁-C₅)-acyl radical ; or the remainder of aspartic acid (D) or leucine (L);
R⁵ represents (C₁-C₈)-alkyl-; (C₁-C₈)-alkyl-C(O)O—(C₁-C₈)-alkyl-; (C₁-C₈)-alkyl-S(O)(O)—(C₁-C₈)-alkyl-; (C₅-C₁₄)—Ar—; (C₅-C₁₄)—Ar—(C₁-C₄)-alkyl-; (C₅-C₁₄)—Ar—C(O)O—(C₁-C₄)-alkyl-; (C₃-C₁₂)-cycloalkyl-; (C₃-C₁₂)-cycloalkyl-(C₁-C₄)-alkyl-; bicycloalkyl-(C₁-C₄)-alkyl-; tricycloalkyl-(C₁-C₄)-alkyl-; said Ar, alkyl, cycloalkyl, bicycloalkyl and tricycloalkyl radicals being unsubstituted or substituted by one or more R⁹ groups;
R⁶ represents a hydrogen atom; a hydroxyl; nitro; (C₁-C₆)-alkyl-O—C(O)—; (C₁-C₆)-alkyl-O—C(O)O— group;
R⁷ and R⁸, independently of one another represent a hydrogen atom or a (C₁-C₆)-alkyl radical unsubstituted or substituted with R⁹;
R⁹ represents halogen; amino; nitro; hydroxyl; (C₁-C₄)alkoxy; (C₁-C4)alkylthio; carboxy; (C₁-C₄)alkyloxycarbonyl; (C₁-C₈)alkyl unsubstituted or substituted with one or more halogen atoms; (C₅-C₁₄)Ar; (C₅-C₁₄)Ar—(C₁-C₄)alkyl;
or one or more isomers of a compound having Formula I, alone or in a mixture, a pharmaceutically acceptable salt, ester, hydrate, solvate, clathrate, enantiomer, or polymorph thereof.

17. The method of claim 16, wherein the composition further comprises a VEGF-A inhibitor.

18. The method of claim 16, wherein the composition is injected into the vitreous of the affected eye.

19. The method of claim 16, wherein the composition is formed into an ophthalmic device and the device is implanted in the posterior segment of the affected eye.

20. The method of claim 16, wherein the VEGF-A inhibitor comprises an anti-VEGF-A antibody.

21. The method of claim 20, wherein the VEGF-A inhibitor comprises bevacizumab, ranibizumab, or a combination thereof.

22. The method of claim 16, wherein the VEGF-A inhibitor comprises a VEGF-A aptamer.

23. The method of claim 16, wherein the composition further comprises an anti-inflammatory agent.

24. The method of claim 23, wherein the anti-inflammatory agent comprises a material selected from the group consisting of NSAIDs, PPAR ligands, combinations thereof, and mixtures thereof.

25. The method of claim 16, wherein the integrin or vitronectin receptor antagonist and the VEGF-A inhibitor are present in the composition in a total amount sufficient to be effective for said treating, reducing, ameliorating, alleviating, or inhibiting progression of, said pathological ocular neovascularization.

26. The method of claim 25, wherein the pathological ocular neovascularization has an etiology in inflammation.

27. The method of claim 26, wherein the pathological ocular neovascularization is selected from the group consisting of diabetic retinopathy (“DR”), dry age-related macular degeneration (“AMD”), wet AMD, diabetic macular edema (“DME”), retinal detachment, posterior uveitis, corneal neovascularization, iris neovascularization, and combinations thereof.

28. The method of claim 27, wherein the pathological ocular neovascularization is wet AMD.

29. The method of claim 27, wherein the pathological ocular neovascularization is diabetic retinopathy.

30. A compound having anyone of Formulae II-X, a free acid thereof, a pharmaceutically acceptable salt, ester, hydrate, solvate, clathrate, enantiomer, or polymorph thereof.

31. The compound of claim 30, which has Formula IV, a free acid thereof, a pharmaceutically acceptable salt, ester, hydrate, solvate, clathrate, enantiomer, or polymorph thereof.
